(19) United States
(12) Patent Application Publication
(10) Pub. No.: US 2012/0264755 A1
(43) Pub. Date: Oct. 18, 2012

(54) PYRIDINYL DERIVATIVES AS INHIBITORS OF ENZYME NICOTINAMIDE PHOSPHORIBOSYLTRANSFERASE

(75) Inventors: Mette K. Christensen, Holte (DK); Fredrik Bjorkling, Helsingborg (SE)

(73) Assignee: TopoTarget, Copenhagen 0 (DE)

(21) Appl. No.: 13/377,408

(22) PCT Filed: Jun. 9, 2010
(86) PCT No.: PCT/EP2010/058102

§ 371 Date: Jun. 20, 2012

Related U.S. Application Data
(60) Provisional application No. 61/185,281, filed on Jun. 9, 2009.

(51) Int. Cl. A61K 31/5377; C07D 405/12; A61K 31/4433; C07D 213/72; A61K 31/44; A61K 31/5355; A61P 29/00; A61P 3/10; A61P 19/02; A61P 1/00; A61P 11/06; A61P 11/00; A61P 19/10; A61P 17/00; A61P 17/06; A61P 37/06; A61P 25/28; A61P 9/10; A61P 35/00; A61P 35/02; A61P 31/12; A61P 31/18; C07D 413/12

(52) U.S. Cl. 514/237.2; 544/124; 546/282.1; 514/336; 546/306; 514/353; 544/63; 514/228.8

(57) ABSTRACT

The present application discloses a compound of the formula (I) wherein Q is optionally substituted pyridyl; p is 0-6. Y is formulae (i), (ii) and (iii) where X is ═O, ═S and ═N—CN, r is 1-12, R is —Z-A, Z is a single bond, —S(═O)₂—, >P═O, >C═O, —C(═O)NH—, and —C(═S)NH—; and A is hydrogen, C₁₋₁₂-alkyl, C₃₋₁₂-cycloalkyl, —[CH₂CH₂O]₁₋₁₀—(C₁₋₆-alkyl), C₁₋₁₂-alkenyl, aryl, heterocyclyl, and heteroaryl; B is a single bond, —NR^{N}—, —S(═O)₂— and —O—; wherein R^{N} is selected from hydrogen, C₁₋₁₂-alkyl, C₃₋₁₂-cycloalkyl, —[CH₂CH₂O]₁₋₁₀—(C₁₋₆-alkyl), C₁₋₁₂-alkenyl, aryl, heterocyclyl, and heteroaryl; s is 0-6; and Cy is aryl, cycloalkyl, heterocyclyl, and heteroaryl. The compounds are useful for use as a medicament for the treatment of a disease or a condition caused by an elevated level of nicotinamide phosphoribosyltransferase (NAMPRT).

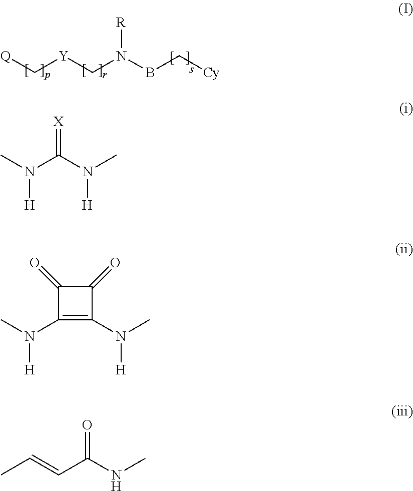

### FIELD OF THE INVENTION

The present invention relates to pyridinyl derivatives which are useful for the inhibiting of the enzyme nicotinamide phosphoribosyltransferase (NAMPRT), and to medical use of such pyridinyl derivatives.

### BACKGROUND OF THE INVENTION

Inhibition of the enzyme nicotinamide phosphoribosyltransferase (NAMPRT) results in the inhibition of NF-kB, the inhibition of NF-kB being a result of the lowering of cellular concentrations of nicotinamide adenine dinucleotide (NAD) (Beauparlant et al (2007) AACR-NCI-EORTC International Conference on Molecular Targets and Cancer Therapeutics, 2007 Oct. 22-26 Abstract nr A82; and Roulson et al (2007) AACR-NCI-EORTC International Conference on Molecular Targets and Cancer Therapeutics, 2007 Oct. 22-26 Abstract nr A81). Tumor cells have elevated expression of NAMPRT and a high rate of NAD turnover due to high ADP-ribosylation activity required for DNA repair, genome stability, and telomere maintenance making them more susceptible to NAMPRT inhibition than normal cells. This also provides a rationale for the use of compounds of this invention in combination with DNA damaging agents for future clinical trials.

The pathways of NAD biosynthesis are shown in FIG. 1.

NAMPRT is involved in the biosynthesis of nicotinamide adenine dinucleotide (NAD) and NAD(P). NAD can be synthesized in mammalian cells by three different pathways starting either from tryptophan via quinolinic acid, from nicotinic acid (niacin) or from nicotinamide (niacinamide).

Quinolinic acid reacts with phosphoribosyl pyrophosphate to form niacin mononucletide (dNAM) using the enzyme quinolinic acid phosphoribosyltransferase \textcircled{3} which is found in liver kidney and brain.

Nicotinic acid (niacin) reacts with PRPP to form niacin mononucleotide (dNAM), using the enzyme niacin phosphoribosyltransferase \textcircled{2} which is widely distributed in various tissues.

Nicotinamide (niacinamide) reacts with PRPP to give niacinamide mononucleotide (NAM) using the enzyme nicotinamide phosphoribosyltransferase (NAMPRT) \textcircled{1} which is also widely distributed in various tissues.

The subsequent addition of adenosine monophosphate to the mononucleotides results in the formation of the corresponding dinucleotides: Niacin mononucleotide and niacinamide mononucleotide react with ATP to form niacin adenine dinucleotide (dNAD) and niacinamide adenine dinucleotide (NAD) respectively. Both reactions, although they take place on different pathways, are catalysed by the same enzyme, NAD pyrophosphorylase \textcircled{4}.

A further amidation step is required to convert niacin adenine dinucleotide (dNAD) to niacinamide adeinine dinucleotide (NAD) The enzyme which catalyses this reaction is NAD synthetase \textcircled{5}. NAD is the immediate precursor of niacinamide adenine dinucleotide phosphate (NAD(P)) The reaction is catalysed by NAD kinase. For details see, e.g., Cory J. G. Purine and pyrimidine nucleotide metabolism In: Textbook of Biochemistry and Clinical Correlations 3^{rd} edition ed. Devlin, T, Wiley, Brisbane 1992, pp 529-574.

Normal cells can typically utilize both precursors niacin and niacinamide for NAD(P) synthesis, and in many cases additionally tryptophan or its metabolites. Accordingly, murine glial cells use niacin, niacinamide and quinolinic acid (Grant et al. (1998) J. Neurochem. 70: 1759-1763). Human lymphocytes use niacin and niacinamide (Carson et al (1987) J. Immunol. 138: 1904-1907; Berger et al (1982) Exp. Cell Res. 137; 79-88). Rat liver cells use niacin, niacinamide and tryptophan (Yamada et al (1983) Int. J. Vit. Nutr. Res. 53: 184-1291; Shin et al (1995) Int. J. Vit. Nutr. Res. 65: 143-146; Dietrich (1971) Methods Enzymol. 18B; 144-149). Human erythrocytes use niacin and niacinamide (Rocchigiani et al (1991) Purine and pyrimidine metabolism in man VII Part B ed. Harkness et al Plenum Press New York pp 337-3490). Leukocytes of guinea pigs use niacin (Flechner et al (1970), Life Science 9: 153-162).

NAD(P) is involved in a variety of biochemical reactions which are vital to the cell and have therefore been thoroughly investigated. The role of NAD(P) in the development and growth of tumours has also been studied. It has been found that many tumour cells utilize niacinamide for cellular NAD(P) synthesis. Niacin and tryptophan which constitute alternative precursors in many normal cell types cannot be utilized in tumour cells, or at least not to an extent sufficient for cell survival. Selective inhibition of an enzyme which is only on the niacinamide pathway (such as NAMPRT) would constitute a method for the selection of tumour specific drugs. This has been exemplified by the NAMPRT inhibitor APO866. (see Hasmann and Schemainda, Cancer Res 63(21):7463-7442.)

It is known that various derivatives of pyridine substituted in a specific manner have pharmacologically useful properties, putatively by inhibition of NAMPRT. Such compounds are described in the following published patent applications: WO 2006/066584, WO 2003/097602, WO 2003/097601, WO 2002/094813, WO 2002/094265, WO 2002/042265, WO 1997/048695, WO 1997/048696, WO 1997/048397, WO 1999/031063, WO 1999/031060 and WO 1999/031087. All of these compounds however are structurally distinct from the compounds of the present invention.

### SUMMARY OF THE INVENTION

It is believed that the novel compounds of the invention are acting on the enzyme nicotinamide phosphoribosyltransferase (NAMPRT), and that the down-stream inhibition of NF-kB is the result of the lowering of cellular concentrations of nicotinamide adenine dinucleotide (NAD).

Hence, the present invention provides compounds of the general formula (I) according to claim 1, and the utilization of these compounds in medicine, cf. claims 13-19.

Inhibitors of the enzyme NAMPRT may be used in the treatment of cancer (WO 1997/48696), to cause immuno-suppression (WO 1997/48397), for the treatment of diseases involving angiogenesis (WO 2003/80054), for the treatment of rheumatoid arthritis or septic shock (WO 2008/025857), for the prophylaxis and treatment of ischaemia (PCT/EP2009/052572 [unpublished application]) or for the prophylaxis and treatment of diabetic nephropathy (Song et al. [2008] Am J Physiol Renal Physiol 295:F1485-F1494])

### BRIEF DESCRIPTION OF THE FIGURE

FIG. 1 illustrates the pathway of NAD biosynthesis (from Biedermann E. et al, WO 00/50399).

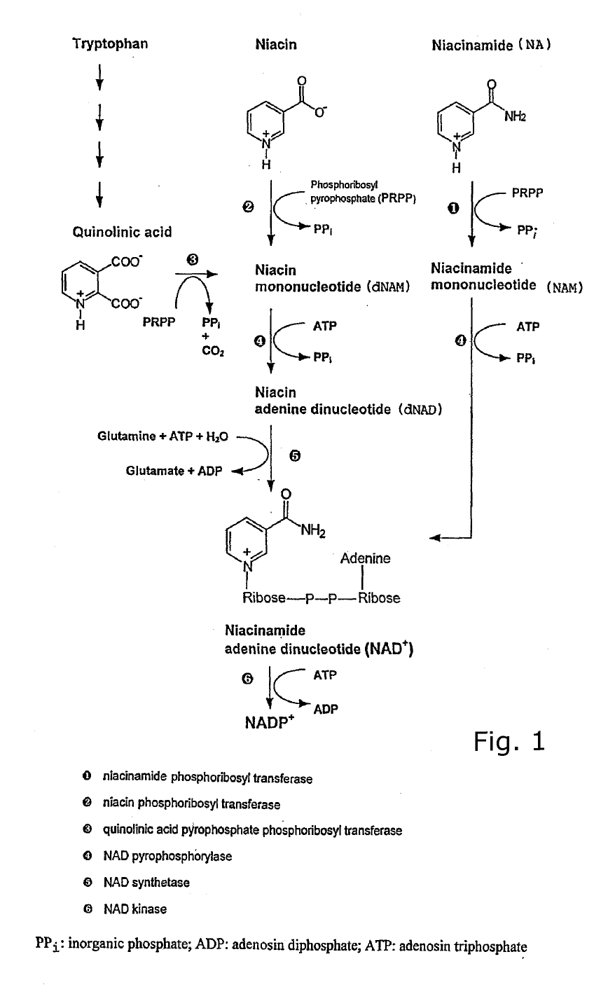

### DETAILED DISCLOSURE OF THE INVENTION

### Compounds of the Invention

The present invention i.a. relates to particular pyridinyl derivatives which are useful for the inhibition of the enzyme nicotinamide phosphoribosyltransferase (NAMPRT).

The present invention relates to compounds of the formula (I)

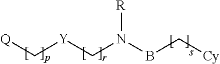

wherein
Q is selected from optionally substituted pyrid-3-yl and optionally substituted pyrid-4-yl;
p is an integer of 0-6;
Y is selected from (i)-(iii):

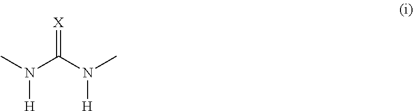

where X is selected from ═O, ═S and ═N—CN,

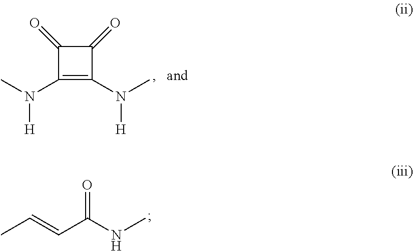

r is an integer of 1-12,
R designates —Z-A, wherein Z is selected from a single bond, —S(═O)₂—, >P═O, >C═O, —C(═O)NH—, and —C(═S)NH—; and A is selected from hydrogen, optionally substituted C₁₋₁₂-alkyl, optionally substituted C₃₋₁₂-cycloalkyl, —[CH₂CH₂O]₁₋₁₀-(optionally substituted C₁₋₆-alkyl), optionally substituted C₁₋₁₂-alkenyl, optionally substituted aryl, optionally substituted heterocyclyl, and optionally substituted heteroaryl;
B is selected from a single bond, —NR^{N}—, —S(═O)₂— and —O—; wherein R^{N} is selected from hydrogen, optionally substituted C₁₋₁₂-alkyl, optionally substituted C₃₋₁₂-cycloalkyl, —[CH₂CH₂O]₁₋₁₀-(optionally substituted C₁₋₆-alkyl), optionally substituted C₁₋₁₂-alkenyl, optionally substituted aryl, optionally substituted heterocyclyl, and optionally substituted heteroaryl;
s is an integer of 0-6; and
Cy is selected from optionally substituted aryl, optionally substituted cycloalkyl, optionally substituted heterocyclyl, and optionally substituted heteroaryl.

### DEFINITIONS

In the present context, the terms “C₁₋₁₂-alkyl” and “C₁₋₆-alkyl” are intended to mean a linear, cyclic or branched hydrocarbon group having 1 to 12 carbon atoms and 1 to 6 carbon atoms, respectively, such as methyl, ethyl, propyl, iso-propyl, cyclopropyl, butyl, iso-butyl, tert-butyl, cyclobutyl, pentyl, cyclopentyl, hexyl, and cyclohexyl.

Although the term “C₃₋₁₂-cycloalkyl” and “C₃₋₈-cycloalkyl” are encompassed by the term “C₁₋₁₂-alkyl”, it refers specifically to the mono- and bicyclic counterparts, including alkyl groups having exo-cyclic atoms, e.g. cyclohexyl-methyl.

Similarly, the terms “C₂₋₁₂-alkenyl” and “C₂₋₆-alkenyl” are intended to cover linear, cyclic or branched hydrocarbon groups having 2 to 12 carbon atoms and 2 to 6 carbon atoms, respectively, and comprising (at least) one unsaturated bond. Examples of alkenyl groups are vinyl, allyl, butenyl, pentenyl, hexenyl, heptenyl, octenyl, heptadecaenyl. Preferred examples of alkenyl are vinyl, allyl, butenyl, especially allyl.

Although the term “C₃₋₁₂-cycloalkenyl” is encompassed by the term “C₂₋₁₂-alkenyl”, it refers specifically to the mono- and bicyclic counterparts, including alkenyl groups having exo-cyclic atoms, e.g. cyclohexenyl-methyl and cyclohexyl-allyl.

In the present context, i.e. in connection with the terms “alkyl”, “cycloalkyl”, “alkoxy”, “alkenyl”, “cycloalkenyl” and the like, the term “optionally substituted” is intended to mean that the group in question may be substituted one or several times, preferably 1-3 times, with group(s) selected from hydroxy (which when bound to an unsaturated carbon atom may be present in the tautomeric keto form), C₁₋₆-alkoxy (i.e. C₁₋₆-alkyloxy), C₂₋₆-alkenyloxy, carboxy, oxo (forming a keto or aldehyde functionality), C₁₋₆-alkoxycarbonyl, C₁₋₆-alkylcarbonyl, formyl, aryl, aryloxy, arylamino, arylcarbonyl, aryloxycarbonyl, arylcarbonyloxy, arylaminocarbonyl, arylcarbonylamino, heteroaryl, heteroaryloxy, heteroarylamino, heteroarylcarbonyl, heteroaryloxycarbonyl, heteroarylcarbonyloxy, heteroarylaminocarbonyl, heteroarylcarbonylamino, heterocyclyl, heterocyclyloxy, heterocyclylamino, heterocyclylcarbonyl, heterocyclyloxycarbonyl, heterocyclylcarbonyloxy, heterocyclylaminocarbonyl, heterocyclylcarbonylamino, amino, mono- and di(C₁₋₆-alkyl)amino, —N(C₁₋₄-alkyl)₃⁺, carbamoyl, mono- and di(C₁₋₆-alkyl)-aminocarbonyl, C₁₋₆-alkylcarbonylamino, cyano, guanidino, carbamido, C₁₋₆-alkyl-sulphonyl-amino, aryl-sulphonyl-amino, heteroaryl-sulphonyl-amino, C₁₋₆-alkanoyloxy, C₁₋₆-alkyl-sulphonyl, C₁₋₆-alkyl-sulphinyl, C₁₋₆-alkylsulphonyloxy, nitro, C₁₋₆-alkylthio, and halogen, where any aryl, heteroaryl and heterocyclyl may be substituted as specifically described below for aryl, heteroaryl and heterocyclyl, and any alkyl, alkoxy, and the like, representing substituents may be substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s).

Typically, the substituents are selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo (forming a keto or aldehyde functionality), C₁₋₆-alkylcarbonyl, formyl, aryl, aryloxy, arylamino, arylcarbonyl, heteroaryl, heteroaryloxy, heteroarylamino, heteroarylcarbonyl, heterocyclyl, heterocyclyloxy, heterocyclylamino, heterocyclylcarbonyl, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, guanidino, carbamido, C₁₋₆-alkyl-sulphonyl-amino, C₁₋₆-alkyl-sulphonyl, C₁₋₆-alkyl-sulphinyl, C₁₋₆-alkylthio, halogen, where any aryl, heteroaryl and heterocyclyl may be substituted as specifically described below for aryl, heteroaryl and heterocyclyl.

In some embodiments, substituents are selected from hydroxy, C₁₋₆-alkoxy, amino, mono- and di(C₁₋₆-alkyl)amino, carboxy, C₁₋₆-alkylcarbonylamino, C₁₋₆-alkylamino-carbonyl, or halogen.

The term “halogen” includes fluoro, chloro, bromo, and iodo.

In the present context, the term “aryl” is intended to mean a fully or partially aromatic carbocyclic ring or ring system, such as phenyl, naphthyl, 1,2,3,4-tetrahydronaphthyl, anthracyl, phenanthracyl, pyrenyl, benzopyrenyl, fluorenyl and xanthenyl, among which phenyl is a preferred example.

The term “heteroaryl” is intended to mean a fully or partially aromatic carbocyclic ring or ring system where one or more of the carbon atoms have been replaced with heteroatoms, e.g. nitrogen (═N— or —NH—), sulphur, and/or oxygen atoms. Examples of such heteroaryl groups are oxazolyl, isoxazolyl, thiazolyl, isothiazolyl, pyrrolyl, imidazolyl, pyrazolyl, pyridinyl, pyrimidinyl, pyrazinyl, pyridazinyl, triazinyl, coumaryl, furanyl, thienyl, quinolyl, benzothiazolyl, benzotriazolyl, benzodiazolyl, benzooxozolyl, phthalazinyl, phthalanyl, triazolyl, tetrazolyl, isoquinolyl, acridinyl, carbazolyl, dibenzazepinyl, indolyl, benzopyrazolyl, phenoxazonyl. Particularly interesting heteroaryl groups are benzimidazolyl, oxazolyl, isoxazolyl, thiazolyl, isothiazolyl, pyrrolyl, imidazolyl, pyrazolyl, pyridinyl, pyrimidinyl, pyrazinyl, pyridazinyl, furyl, thienyl, quinolyl, triazolyl, tetrazolyl, isoquinolyl, indolyl in particular benzimidazolyl, pyrrolyl, imidazolyl, pyridinyl, pyrimidinyl, furyl, thienyl, quinolyl, tetrazolyl, and isoquinolyl.

The term “heterocyclyl” is intended to mean a non-aromatic carbocyclic ring or ring system where one or more of the carbon atoms have been replaced with heteroatoms, e.g. nitrogen (═N— or —NH—), sulphur, and/or oxygen atoms. Examples of such heterocyclyl groups (named according to the rings) are imidazolidine, piperazine, hexahydropyridazine, hexahydropyrimidine, diazepane, diazocane, pyrrolidine, piperidine, azepane, azocane, aziridine, azirine, azetidine, pyrroline, tropane, oxazinane (morpholine), azepine, dihydroazepine, tetrahydroazepine, and hexahydroazepine, oxazolane, oxazepane, oxazocane, thiazolane, thiazinane, thiazepane, thiazocane, oxazetane, diazetane, thiazetane, tetrahydrofuran, tetrahydropyran, oxepane, tetrahydrothiophene, tetrahydrothiopyrane, thiepane, dithiane, dithiepane, dioxane, dioxepane, oxathiane, oxathiepane. The most interesting examples are tetrahydrofuran, imidazolidine, piperazine, hexahydropyridazine, hexahydropyrimidine, diazepane, diazocane, pyrrolidine, piperidine, azepane, azocane, azetidine, tropane, oxazinane (morpholine), oxazolane, oxazepane, thiazolane, thiazinane, and thiazepane, in particular tetrahydrofuran, imidazolidine, piperazine, hexahydropyridazine, hexahydropyrimidine, diazepane, pyrrolidine, piperidine, azepane, oxazinane (morpholine), and thiazinane.

The term “N-containing heterocyclic or heteroaromatic ring” are intended to encompass those mentioned under “heterocyclyl” and “heteroaryl”, respectively, which include one or more heteroatoms, at least one of which begin a nitrogen atom. Examples hereof are piperazine, isoxazole, isoxazolidine, and morpholine, etc.

The term “N,O-containing heterocyclic or heteroaromatic ring” are intended to encompass those mentioned under “heterocyclyl” and “heteroaryl”, respectively, which include two or more heteroatoms, two of which being neighbouring nitrogen and oxygen atoms. Examples hereof are isoxazole, isoxazolidine, morpholine, etc.

In the present context, i.e. in connection with the terms “pyrid-3-yl”, “pyrid-4-yl”, “aryl”, “heteroaryl”, “heterocyclyl”, “N,O-containing heterocyclic or heteroaromatic ring” and the like (e.g. “aryloxy”, “heterarylcarbonyl”, etc.), the term “optionally substituted” is intended to mean that the group in question may be substituted one or several times, preferably 1-5 times, in particular 1-3 times, with group(s) selected from hydroxy (which when present in an enol system may be represented in the tautomeric keto form), C₁₋₆-alkyl, C₁₋₆-alkoxy, C₂₋₆-alkenyloxy, oxo (which may be represented in the tautomeric enol form), oxide (only relevant as the N-oxide), carboxy, C₁₋₆-alkoxycarbonyl, C₁₋₆-alkylcarbonyl, formyl, aryl, aryloxy, arylamino, aryloxycarbonyl, arylcarbonyl, heteroaryl, heteroarylamino, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, cyano, guanidino, carbamido, C₁₋₆-alkanoyloxy, C₁₋₆-alkyl-sulphonyl-amino, aryl-sulphonyl-amino, heteroaryl-sulphonyl-amino, C₁₋₆-alkyl-suphonyl, C₁₋₆-alkyl-sulphinyl, C₁₋₆-alkylsulphonyloxy, nitro, sulphanyl, amino, amino-sulfonyl, mono- and di(C₁₋₆-alkyl)amino-sulfonyl, dihalogen-C₁₋₄-alkyl, trihalogen-C₁₋₄-alkyl, halogen, where aryl and heteroaryl representing substituents may be substituted 1-3 times with C₁₋₄-alkyl, C₁₋₄-alkoxy, nitro, cyano, amino or halogen, and any alkyl, alkoxy, and the like, representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino.

Typically, the substituents are selected from hydroxy, C₁₋₆-alkyl, C₁₋₆-alkoxy, oxo (which may be represented in the tautomeric enol form), carboxy, C₁₋₆-alkylcarbonyl, formyl, amino, mono- and di(C₁₋₆-alkyl)amino; carbamoyl, mono- and di(C₁₋₆-alkyl)amino-carbonyl, amino-C₁₋₆-alkyl-aminocarbonyl, C₁₋₆-alkylcarbonylamino, guanidino, carbamido, C₁₋₆-alkyl-sulphonyl-amino, aryl-sulphonyl-amino, heteroaryl-sulphonyl-amino, C₁₋₆-alkyl-suphonyl, C₁₋₆-alkyl-sulphinyl, C₁₋₆-alkylsulphonyloxy, sulphanyl, amino, amino-sulfonyl, mono- and di(C₁₋₆-alkyl)amino-sulfonyl or halogen, where any alkyl, alkoxy and the like, representing substituents may be substituted with hydroxy, C₁₋₆-alkoxy, C₂₋₆-alkenyloxy, amino, mono- and di(C₁₋₆-alkyl)amino, carboxy, C₁₋₆-alkyl-carbonylamino, halogen, C₁₋₆-alkylthio, C₁₋₆-alkyl-sulphonyl-amino, or guanidino. In some embodiments, the substituents are selected from C₁₋₆-alkyl, C₁₋₆-alkoxy, amino, mono- and di(C₁₋₆-alkyl)amino, sulphanyl, carboxy or halogen, where any alkyl, alkoxy and the like, representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino.

Groups (e.g. A) including C₃₋₁₂-cycloalkyl, C₃₋₁₂-cycloalkenyl and/or aryl as at least a part of the substituent are said to include “a carbocyclic ring”.

Groups (e.g. A) including heterocyclyl or heteroaryl as at least a part of the substituent are said to include “a heterocyclic ring” and “a heteroaromatic ring”, respectively.

The term “pharmaceutically acceptable salts” is intended to include acid addition salts and basic salts. Illustrative examples of acid addition salts are pharmaceutically acceptable salts formed with non-toxic acids. Exemplary of such organic salts are those with maleic, fumaric, benzoic, ascorbic, succinic, oxalic, bis-methylenesalicylic, methanesulfonic, ethanedisulfonic, acetic, propionic, tartaric, salicylic, citric, gluconic, lactic, malic, mandelic, cinnamic, citraconic, aspartic, stearic, palmitic, itaconic, glycolic, p-aminobenzoic, glutamic, benzenesulfonic, and theophylline acetic acids, as well as the 8-halotheophyllines, for example 8-bromotheophylline. Exemplary of such inorganic salts are those with hydrochloric, hydrobromic, sulfuric, sulfamic, phosphoric, and nitric acids. Examples of basic salts are salts where the (remaining) counter ion is selected from alkali metals, such as sodium and potassium, alkaline earth metals, such as calcium, and ammonium ions (⁺N(R)₃R′, where R and R′ independently designates optionally substituted C₁₋₆-alkyl, optionally substituted C₂₋₆-alkenyl, optionally substituted aryl, or optionally substituted heteroaryl). Pharmaceutically acceptable salts are, e.g., those described in Remington's Pharmaceutical Sciences, 17. Ed. Alfonso R. Gennaro (Ed.), Mack Publishing Company, Easton, Pa., U.S.A., 1985 and more recent editions and in Encyclopedia of Pharmaceutical Technology. Thus, the term “an acid addition salt or a basic salt thereof” used herein is intended to comprise such salts. Furthermore, the compounds as well as any intermediates or starting materials may also be present in hydrate form.

The term “prodrug” used herein is intended to mean a compound which—upon exposure to physiological conditions—will liberate a derivative of said compound which then will be able to exhibit the desired biological action. Typical examples are labile esters (i.e. a latent hydroxyl group or a latent acid group).

Moreover, it should be understood that the compounds may be present as racemic mixtures or the individual stereoisomers such as enantiomers or diastereomers. The present invention encompasses each and every of such possible stereoisomers (e.g. enantiomers and diastereomers) as well as racemates and mixtures enriched with respect to one of the possible stereoisomers.

### Embodiments

Q is selected from optionally substituted pyrid-3-yl and optionally substituted pyrid-4-yl.

In one primary embodiment, Q is optionally substituted pyrid-3-yl, in particular pyrid-3-yl.

In another embodiment, Q is optionally substituted pyrid-4-yl, in particular pyrid-4-yl.

The integer “p” determines the spatial orientation and the mobility of the substituent Q relative to the group Y, and is an integer of 0-6. In the currently preferred embodiments, p is an integer of 0-3, such as an integer of 0-2, in particular an integer of 0-1, such as 0 or such as 1.

Y is selected from the groups (i)-(iii):

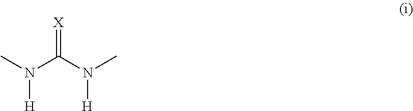

where X is selected from ═O, ═S and ═N—CN,

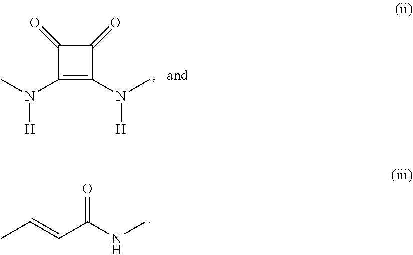

The groups (i)-(iii) representing Y provides somewhat different spatial orientations of the attached substituents, and renders it possible to adjust the overall flexibility of the molecule.

In some currently most interesting embodiments, p is an integer of 0 when Y is a group of the type (ii) or (iii), and an integer of 0-1 when Y is a group of the type (i).

The integer “r” reflects the via-bond distance between the group Y and the nitrogen atom to which the group R (i.e. —Z-A) is attached. Typically, r is an integer of 1-12, and in currently most interesting embodiments, r is an integer of 4-10, in particular 5-9, most preferably 6-8.

R designates —Z-A, wherein Z is selected from a single bond, —S(═O)₂—, >P═O, >C═O, —C(═O)NH—, and —C(═S)NH—, in particular from a single bond and —S(═O)₂—, and A is selected from hydrogen, optionally substituted C₁₋₁₂-alkyl, optionally substituted C₃₋₁₂-cycloalkyl, —[CH₂CH₂O]₁₋₁₀-(optionally substituted C₁₋₆-alkyl), optionally substituted C₁₋₁₂-alkenyl, optionally substituted aryl, optionally substituted heterocyclyl, and optionally substituted heteroaryl.

In one currently particularly relevant embodiment, Z is a single bond, and A is optionally substituted C₃₋₈-cycloalkyl, such as cyclopentyl or cyclohexyl.

In other interesting embodiments, Z is sulfonyl, and A is selected from optionally substituted C₃₋₈-cycloalkyl and optionally substituted C₁₋₆-alkyl, such as cyclopentyl, cyclohexyl, optionally substituted benzyl (e.g. benzyl), or linear or branched C₁₋₆-alkyl.

In another interesting series of embodiments, Z is sulfonyl, and A is optionally substituted aryl, particularly optionally substituted phenyl, e.g. phenyl.

Based on the current set of data, it appears that the variants in which r is an integer of 7-10, such as 8-9, are the most promising, when Z is a single bond, whereas the variants where r is 6-9 are the most promising when Z is —S(═O)₂—.

B is selected from a single bond, —NR^{N}—, —S(═O)₂ and —O—, in particular from a single bond and —O—; wherein R^{N} is selected from hydrogen, optionally substituted C₁₋₁₂-alkyl, optionally substituted C₃₋₁₂-cycloalkyl, —[CH₂CH₂O]₁₋₁₀-(optionally substituted C₁₋₆-alkyl), optionally substituted C₁₋₁₂-alkenyl, optionally substituted aryl, optionally substituted heterocyclyl, and optionally substituted heteroaryl, in particular R^{N} is hydrogen. In some of the most promising embodiments, B is a single bond, and in other embodiments, B is —O—.

The integer “s” determines the spatial orientation and the mobility of the substituent Cy relative to the group N—B—, and is an integer of 0-6. In some embodiments, s is an integer of 0-4, such 0-3. In some embodiments where B is a single bond, s is preferably 1-5, such as 2-4, in particular 3. In some embodiments where B is —O—, s is preferably 0-2, such as 0 or 1.

In some interesting embodiments, when p is 0, and B is a single bond, s is 2-6.

Cy is typically selected from optionally substituted aryl, optionally substituted cycloalkyl, optionally substituted heterocyclyl, and optionally substituted heteroaryl.

In some interesting embodiments, Cy is selected from optionally substituted heterocyclyl, particularly pyran-2-yl or morpholinyl.

In further embodiments, Cy is selected from optionally substituted aryl, particularly phenyl.

This being said, currently very interesting compounds of the formula (I) are those listed in the following:
2-cyano-1-(7-(cyclohexyl(3-morpholinopropyl)amino)octyl)-3-(pyridin-4-yl)guanidine,
2-cyano-1-(7-(cyclohexyl(3-morpholinopropyl)amino)heptyl)-3-(pyridin-4-yl)guanidine,
2-cyano-1-(6-(cyclohexyl(3-morpholinopropyl)amino)hexyl)-3-(pyridin-4-yl)guanidine,
1-(8-(cyclohexyl(3-morpholinopropyl)amino)octyl)-3-(pyridin-3-ylmethyl)urea,
1-(7-(cyclohexyl(3-morpholinopropyl)amino)heptyl)-3-(pyridin-3-ylmethyl)urea,
3-(8-(cyclohexyl(3-morpholinopropyl)amino)-4-(pyridin-4-ylamino)cyclobut-3-ene-1,2-dione,
3-(7-cyclohexyl(3-morpholinopropyl)amino)heptylamino)-4-(pyridin-4-ylamino)cyclobut-3-ene-1,2-dione,
N-(8-(2-cyano-3-(pyridin-4-yl)guanidino)octyl)-N-(3-morpholinopropyl)cyclopentane-sulfonamide,
N-(8-(2-cyano-3-(pyridin-4-yl)guanidino)octyl)-N-(3-morpholinopropyl)cyclohexane-sulfonamide,
N-(7-(2-cyano-3-(pyridin-4-yl)guanidino)heptyl)-N-(3-morpholino propyl)cyclohexane-sulfonamide,
N-(7-(2-cyano-3-(pyridin-4-yl)guanidino)heptyl)-N-(3-morpholino propyl)cyclopentane-sulfonamide,
N-(3-morpholinopropyl)-N-(8-(3-(pyridin-3-ylmethyl)ureido)octyl)cyclopentane-sulfonamide,
N-(3-morpholinopropyl)-N-(8-(3-(pyridin-3-ylmethyl)ureido)octyl)cyclohexane-sulfonamide,
N-(3-morpholinopropyl)-N-(7-(3-(pyridin-3-ylmethyl)ureido)heptyl)cyclohexane-sulfonamide,
N-(3-morpholinopropyl)-N-(7-(3-(pyridin-3-ylmethyl)ureido)heptyl)cyclopentane-sulfonamide,
N-(8-(3,4-dioxo-2-(pyridin-4-ylamino)cyclobut-1-enylamino)octyl)-N-(3-morpholino-propyl)cyclopentanesulfonamide,
N-(8-(3,4-dioxo-2-(pyridin-4-ylamino)cyclobut-1-enylamino)octyl)-N-(3-morpholino-propyl)cyclohexanesulfonamide,
N-(7-(3,4-dioxo-2-(pyridin-4-ylamino)cyclobut-1-enylamino)heptyl)-N-(3-morpholino-propyl)cyclohexanesulfonamide,
N-(7-(3,4-dioxo-2-(pyridin-4-ylamino)cyclobut-1-enylamino)heptyl)-N-(3-morpholino-propyl)cyclopentanesulfonamide,
N-(benzyloxy)-N-(8-(2-cyano-3-pyridin-4-yl)guanidino)octyl)methanesulfonamide,
N-(Benzyloxy)-N-(8-(3,4-dioxo-2-(pyridin-4-ylamino)cyclobute-1-enylamino)octyl)-methansulfonamide,
N-(Benzyloxy)-N-(8-(3-pyridin-3-ylmethyl)ureido)octyl)methansulfonamide,
N-(8-(N-Benzyloxy)methylsulfonamido)octyl-3-(pyridin-3-yl)acrylamide,
N-(benzyloxy)-N-(8-(2-cyano-3-pyridin-4-yl)guanidino)octyl)propane-2-sulfonamide,
N-(Benzyloxy)-N-(8-(3,4-dioxo-2-(pyridin-4-ylamino)cyclobute-1-enylamino)-octyl)propane-2-sulfonamide,
N-(Benzyloxy)-N-(8-(3-pyridin-3-ylmethyl)ureido)octyl)propane-2-sulfonamide,
N-(8-(N-Benzyloxy)propan-2-ylsulfonamido)octyl)-3-(pyridin-3-yl)acrylamide,
N-(Benzyloxy)-N-(8-(3-pyridin-4-ylureido)octyl)propane-2-sulfonamide,
N-(Benzyloxy)-N-(8-(3-pyridin-4-ylthioureido)octyl)propane-2-sulfonamide,
N-(8-(2-cyano-3-(pyridin-4-yl)guanidino)octyl)-N-(3-morpholinopropyl)methane-sulfonamide,
N-(8-(3,4-dioxo-2-(pyridin-4-ylamino)cyclobute-1-enylamino)octyl)-N-(3-morpholino-propyl)methanesulfonamide,
N-(3-Morpholinopropyl)-N-(8-(3-pyridin-3-ylmethyl)ureido)octyl)methanesulfonamide,
N-(8-(N-(3-morpholinopropyl)methylsulfonamido)octyl)-3-(pyridin-3-yl)acrylamide,
N-(3-Morpholinopropyl)-N-(8-(3-pyridin-4-ylthioureido)octyl)methanesulfonamide,
N-(8-(2-cyano-3-(pyridin-4-yl)guanidino)octyl)-N-(3-morpholinopropyl)benzene-sulfonamide,
N-(8-(3,4-dioxo-2-(pyridin-4-ylamino)cyclobute-1-enylamino)octyl)-N-(3-morpholino-propyl)benzenesulfonamide,
N-(3-Morpholinopropyl)-N-(8-(3-pyridin-3-ylmethyl)ureido)octyl)benzenesulfonamide,
N-(8-(N-(3-morpholinopropyl)phenylsulfonamido)octyl)-3-(pyridin-3-yl)acrylamide,
N-(3-Morpholinopropyl)-N-(8-(3-pyridin-4-ylureido)octyl)benzenesulfonamide,
N-(3-Morpholinopropyl)-N-(8-(3-pyridin-4-ylureido)octyl)benzenesulfonamide,
1-(7-Cyclohexyl(3-morpholinopropyl)amino)heptyl)-3-(pyridin-4-yl)thiourea oxalate,
1-(7-Cyclohexyl(3-morpholinopropyl)amino)heptyl)-3-(pyridin-4-yl)urea oxalate,
(E)-N-(7-Cyclohexyl(3-morpholinopropyl)amino)heptyl)-3-(pyridin-3-yl)acrylamide,
N-(6-(2-Cyano-3-pyridin-4-yl)guanidino)hexyl)-N-(3-morpholinopropyl)cyclopentane-sulfonamide,
N-(6-(3,4-Dioxo-2-(pyridin-4-ylamino)cyclobut-1-enylamino)hexyl-N-(3-morpholino-propyl)cyclopentanesulfonamide,
N-(3-morpholinopropyl)-N-(6-(3-pyridin-3-ylmethyl)ureido)hexyl)cyclopentane-sulfonamide,
(E)-N-(6-(N-(3-morpholinopropyl)cyclopentanesulfonamido)hexyl)-3-(pyridin-3-yl)-acrylamide,
N-(3-morpholinopropyl)-N-(6-(3-pyridin-4-ylureido)hexyl)cyclopentanesulfonamide,
N-(3-morpholinopropyl)-N-(6-(3-pyridin-4-ylthioureido)hexyl)cyclopentanesulfonamide,
N-(6-(2-Cyano-3-pyridin-4-yl)guanidino)hexyl)-N-(3-morpholinopropyl)cyclohexane-sulfonamide,
N-(6-(3,4-Dioxo-2-(pyridin-4-ylamino)cyclobut-1-enylamino)hexyl-N-(3-morpholinopropyl)cyclohexanesulfonamide,
N-(3-morpholinopropyl)-N-(6-(3-pyridin-3-ylmethyl)ureido)hexyl)cyclohexane-sulfonamide,
(E)-N-(6-(N-(3-morpholinopropyl)cyclohexanesulfonamido)hexyl)-3-(pyridin-3-yl)acrylamide,
N-(3-morpholinopropyl)-N-(6-(3-pyridin-4-ylthioureido)hexyl)cyclohexanesulfonamide,
N-(7-(2-Cyano-3-pyridin-4-yl)guanidino)heptyl)-1-phenyl-N-(tetrahydro-2H-pyran-2-yloxy)methanesulfonamide,
N-(7-(3,4-Dioxo-2-(pyridin-4-ylamino)cyclobut-1-enylamino)heptyl)-1-phenyl-N-(tetrahydro-2H-pyran-2-yloxy)methanesulfonamide,
1-Phenyl-N-(7-(3-(pyridin-3-ylmethyl)ureido)heptyl)-N-(tetrahydro-2H-pyran-2-yloxy)methanesulfonamide,
(E)-N-(7-(1-phenyl-N-(tetrahydro-2H-pyran-2-yloxy)methylsulfonamido) heptyl)-3-(pyridin-3-yl)acrylamide,
1-phenyl-N-(7-(3-pyridin-4-ylthioureido)heptyl)-N-(tetrahydro-2H-pyran-2-yloxy)-methanesulfonamide,
N-(7-(2-cyano-3-(pyridin-4-yl)guanidino)heptyl)-N-(cyclohexylmethoxy)ethane-sulfonamide,
N-(cyclohexylmethoxy)-N-(7-(3,4-dioxo-2-(pyridin-4-ylamino)cyclobut-1-enylamino)-heptyl)ethanesulfonamide,
N-(cyclohexylmethoxy)-N-(7-(3-(pyridin-3-ylmethyl)ureido)heptyl)ethanesulfonamide,
(E)-N-(7-(N-(cyclohexylmethoxy)ethylsulfonamido)heptyl)-3-(pyridin-3-yl)acrylamide,
N-(cyclohexylmethoxy)-N-(7-(3-(pyridin-4-yl)ureido)heptyl)ethanesulfonamide,
N-(cyclohexylmethoxy)-N-(7-(3-(pyridin-4-yl)thioureido)heptyl)ethanesulfonamide,
N-(7-(2-cyano-3-(pyridin-4-yl)guanidino)heptyl)-N-(cyclohexyloxy)-4-fluorobenzene-sulfonamide,
N-(cyclohexyloxy)-N-(7-(3,4-dioxo-2-(pyridin-4-ylamino)cyclobut-1-enylamino)heptyl)-4-fluorobenzenesulfonamide,
N-(cyclohexyloxy)-4-fluoro-N-(7-(3-(pyridin-3-ylmethyl)ureido)heptyl)benzene-sulfonamide,
(E)-N-(7-(N-(cyclohexyloxy)-4-fluorophenylsulfonamido)heptyl)-3-(pyridin-3-yl)-acrylamide,
N-(cyclohexyloxy)-4-fluoro-N-(7-(3-(pyridin-4-yl)thioureido)heptyl)benzenesulfonamide,
N-(8-(2-cyano-3-(pyridin-4-yl)guanidino)octyl)-N-(3-morpholinopropyl)benzamide,
N-(8-(3,4-dioxo-2-(pyridin-4-ylamino)cyclobut-1-enylamino)octyl)-N-(3-morpholino-propyl)benzamide,
N-(3-morpholinopropyl)-N-(8-(3-(pyridin-3-ylmethyl)ureido)octyl)benzamide,
(E)-N-(3-morpholinopropyl)-N-(8-(3-(pyridin-3-yl)acrylamido)octyl)benzamide,
N-(3-morpholinopropyl)-N-(8-(3-(pyridin-4-yl)thioureido)octyl)benzamide,
N-(8-(2-cyano-3-(pyridin-3-yl)guanidino)octyl)-N-(3-morpholinopropyl)benzamide,
3-cyclohexyl-1-(3-morpholinopropyl)-1-(8-(3-(pyridin-4-yl)thioureido)octyl)urea,
3-(8-(benzyloxy(ethyl)amino)octylamino)-4-(pyridin-4-ylamino)cyclobut-3-ene-1,2-dione,
N-(3-morpholinopropyl)-N-(7-(3-(pyridin-4-yl)thioureido)heptyl)cyclohexane-sulfonamide oxalate,
1-(8-(benzyloxy(ethyl)amino)octyl)-3-(pyridin-4-yl)thiourea oxalate,
1-(8-(benzyloxy(ethyl)amino)octyl)-3-(pyridin-3-ylmethyl)urea oxalate,
1-(8-(benzyloxy(ethyl)amino)octyl)-3-(pyridin-4-yl)urea,
N-(3-morpholinopropyl)-N-(7-(3-(pyridin-4-yl)ureido)heptyl)cyclohexanesulfonamide,
1-(8-(benzyloxy(ethyl)amino)octyl)-2-cyano-3-(pyridin-4-yl)guanidine,
1-(8-(benzyl(ethoxy)amino)octyl)-2-cyano-3-(pyridin-4-yl)guanidine,
2-Cyano-1-(8-(ethyl(2-morpholinoethoxy)amino)octyl)-3-(pyridin-4-yl)guanidine oxalate,
2-Cyano-1-(8-(3-morpholinopropylamino)octyl)-3-(pyridin-4-yl)guanidine,
2-Cyano-1-(8-((dimethylphosphoryl)(3-morpholinopropyl)amino)octyl)-3-(pyridin-4-yl)-guanidine,
N-(8-(3,4-dioxo-2-(pyridin-4-ylamino)cyclobut-1-enylamino)octyl)-P,P-dimethyl-N-(3-morpholinopropyl)phosphinic amide,
1-(8-((dimethylphosphoryl)(3-morpholinopropyl)amino)octyl)-3-(pyridin-3-ylmethyl)-urea,
(E)-N-(8-((dimethylphosphoryl)(3-morpholinopropyl)amino)octyl)-3-(pyridin-3-yl)-acrylamide,
1-(8-((dimethylphosphoryl)(3-morpholinopropyl)amino)octyl)-3-(pyridin-4-yl)thiourea,
1-(8-(2-cyano-3-(pyridin-4-yl)guanidino)octyl)-3-cyclohexyl-1-(morpholinopropyl)urea,
1-(8-(2-cyano-3-(pyridin-4-yl)guanidino)octyl)-1-(3-morpholinopropyl)-3-phenyl-thiourea,
N-(8-(2-cyano-3-(pyridin-4-yl)guanidino)octyl)-N-(3-morpholinopropyl)hydrazine-carboxamide,
N-(8-(3,4-dioxo-2-(pyridin-4-ylamino)cyclobut-1-enylamino)octyl)-N-(3-morpholino-propyl)hydrazinecarboxamide,
N-(3-morpholinopropyl)-N-(8-(3-(pyridin-3-ylmethyl)ureido)octyl)hydrazinecarboxamide,
N-(7-(2-cyano-3-(pyridin-4-yl)guanidino)heptyl)-N-(2-fluoroethyl)cyclohexansulfonamide,
N-(7-(3,4-dioxo-2-(pyridin-4-ylamino)cyclobut-1-enylamino)heptyl)-N-(2-fluoroethyl)-cyclohexanesulfonamide,
N-(2-fluoroethyl)-N-(7-(3-(pyridin-3-ylmethyl)ureido)heptyl)cyclohexanesulfonamide,
(E)-N-(7-(N-(2-fluoroethyl)cyclohexanesulfonamido)heptyl)-3-(pyridin-3-yl)acrylamide,
N-(2-fluoroethyl)-N-(7-(3-pyridin-4-ylthioureido)heptyl)cyclohexanesulfonamide,
N-(7-(2-cyano-3-(pyridin-4-yl)guanidino)octyl)-N-(2-fluoroethyl)cyclohexansylfonamide,
N-(7-(3,4-dioxo-2-(pyridin-4-ylamino)cyclobut-1-enylamino)octyl)-N-(2-fluoroethyl)-cyclohexanesulfonamide,
(E)-N-(7-(N-(2-fluoroethyl)cyclohexanesulfonamido)octyl)-3-(pyridin-3-yl)acrylamide,
N-(2-fluoroethyl)-N-(7-(3-pyridin-4-ylthioureido)octyl)cyclohexanesulfonamide,
2-cyano-1-(8-(cyclohexylmethoxyamino)octyl)-3-(pyridin-4-yl)guanidine,
N-(8-(2-cyano-3-(pyridin-4-yl)guanidino)octyl)-N-(cyclohexylmethyloxy)-2,2,2-trifluoro-methanesulfonamide,
1-(8-(2-cyano-3-(pyridin-4-yl)guanidino)octyl)-3-cyclohexyl-1-(cyclohexylmethoxy)-thiourea,
2-cyano-1-(8-(cyclohexylmethoxyamino)hexyl)-3-(pyridin-4-yl)guanidine,
2-cyano-1-(8-(cyclohexylmethoxyamino)heptyl)-3-(pyridin-4-yl)guanidine,
N-(6-(2-cyano-3-(pyridin-4-yl)guanidino)hexyl)-N-(cyclohexylmethoxy)-methanesulfonamide,
N-(6-(2-cyano-3-(pyridin-4-yl)guanidino)hexyl)-N-(cyclohexylmethoxy)-2,2,2-trifluoro-ethanesulfonamide,
1-(6-(2-cyano-3-(pyridin-4-yl)guanidino)hexyl)-1-(cyclohexylmethoxy)-3-ethylurea,
1-(6-(2-cyano-3-(pyridin-4-yl)guanidino)hexyl)-1-(cyclohexylmethoxy)-3-isopropylurea,
1-(6-(2-cyano-3-(pyridin-4-yl)guanidino)hexyl)-1-(cyclohexylmethoxy)-3-methyl-thiourea,
1-(6-(2-cyano-3-(pyridin-4-yl)guanidino)hexyl)-3-cyclohexyl-1-(cyclohexylmethoxy)-thiourea,
N-(7-(2-cyano-3-(pyridin-4-yl)guanidino)heptyl)-N-(cyclohexylmethoxy)methane-sulfonamide,
N-(7-(2-cyano-3-(pyridin-4-yl)guanidino)heptyl)-N-(cyclohexylmethoxy)-2,2,2-trifluoroethanesulfonamide,
1-(7-(2-cyano-3-(pyridin-4-yl)guanidino)heptyl)-N-(cyclohexylmethoxy)-3-ethylurea,
1-(7-(2-cyano-3-(pyridin-4-yl)guanidino)heptyl)-N-(cyclohexylmethoxy)-3-isopropyl-urea,
1-(7-(2-cyano-3-(pyridin-4-yl)guanidino)heptyl)-N-(cyclohexylmethoxy)-3-methylthiourea,
1-(7-(2-cyano-3-(pyridin-4-yl)guanidino)heptyl)-3-cyclohexyl-1-(cyclohexylmethoxy)-thiourea,
N-(8-(2-cyano-3-(pyridin-4-yl)guanidino)octyl)-N-(cyclohexylmethoxy)methane-sulfonamide,
1-(8-(2-cyano-3-(pyridin-4-yl)guanidino)octyl)-N-(cyclohexylmethoxy)-3-ethylurea,
1-(8-(2-cyano-3-(pyridin-4-yl)guanidino)octyl)-N-(cyclohexylmethoxy)-3-isopropylurea,
1-(8-(2-cyano-3-(pyridin-4-yl)guanidino)octyl)-N-(cyclohexylmethoxy)-3-methyl-thiourea,
N-(6-(2-cyano-3-(pyridin-4-yl)guanidino)hexyl)-N-(2-fluoroethyl)cyclohexansylfonamide,
N-(6-(3,4-dioxo-2-(pyridin-4-ylamino)cyclobut-1-enylamino)hexyl)-N-(2-fluoroethyl)-cyclohexanesulfonamide,
(E)-N-(6-(N-(2-fluoroethyl)cyclohexanesulfonamido)hexyl)-3-(pyridin-3-yl)acrylamide,
N-(2-fluoroethyl)-N-(6-(3-pyridin-4-ylthioureido)hexyl)cyclohexanesulfonamide,
2-cyano-1-(7-morpholinoheptyl)-3-(pyridin-4-yl)guanidine,
3-(7-morpholinoheptylamino)-4-(pyridin-4-ylamino)cyclobut-3-ene-1,2-dione,
1-(7-morpholinoheptylamino)-3-(pyridin-3-ylmethyl)urea,
(E)-N-(7-morpholinoheptyl)-3-(pyridin-3-yl)acrylamide,
1-(7-morpholinoheptyl)-3-(pyridin-4-yl)thiourea,
N-(6-(2-cyano-3-(pyridin-4-yl)guanidino)hexyl)-N-(cyclohexylmethoxy)propane-2-sulfonamide,
N-(6-(2-cyano-3-(pyridin-4-yl)guanidino)hexyl)-N-(cyclohexylmethoxy)ethane-2-sulfonamide,
N-(6-(2-cyano-3-(pyridin-4-yl)guanidino)hexyl)-N-(cyclohexylmethoxy)cyclopropane-2-sulfonamide,
N-(6-(2-cyano-3-(pyridin-4-yl)guanidino)hexyl)-N-(cyclohexylmethoxy)-1,1,1-trifluoromethanesulfonamide,
2-cyano-1-(5-(cyclohexylmethoxyamino)pentyl)-3-(pyridin-4-yl)guanidine,
3-(5-(cyclohexylmethoxyamino)pentylamino)-4-(pyridin-4-ylamino)cyclobut-3-ene-1,2-dione,
N-(5-(2-cyano-3-(pyridin-4-yl)guanidino)pentyl)-N-cyclohexylmethoxy)methane-sulfonamide,
N-(5-(2-cyano-3-(pyridin-4-yl)guanidino)pentyl)-N-cyclohexylmethoxy)ethane-sulfonamide,
1-(5-(2-cyano-3-(pyridin-4-yl)guanidino)pentyl)-1-(cyclohexylmethoxy)-3-isopropyl-urea,
1-(5-(2-cyano-3-(pyridin-4-yl)guanidino)pentyl)-1-(cyclohexylmethoxy)-3-ethylurea,
1-(5-(2-cyano-3-(pyridin-4-yl)guanidino)pentyl)-1-(cyclohexylmethoxy)-3-methyl-thiourea,
N-(5-(2-cyano-3-(pyridin-4-yl)guanidino)pentyl)-N-(cyclohexylmethoxy)benzene-sulfonamide,
N-(5-(2-cyano-3-(pyridin-4-yl)guanidino)pentyl)-N-(cyclohexylmethoxy)propane-2-sulfonamide,
N-(benzyloxy)-N-(8-(3-(pyridin-4-yl)ureido)octyl)methanesulfonamide,
N-(benzyloxy)-N-(8-(3-(pyridin-4-yl)thioureido)octyl)methanesulfonamide,
N-(benzyloxy)-N-(6-(2-cyano-3-(pyridin-4-yl)guanidino)hexyl)methanesulfonamide,
N-(benzyloxy)-N-(6-(3,4-dioxo-2-(pyridin-4-ylamino)cyclobut-1-enylamino)hexyl)-methanesulfonamide,
N-(benzyloxy)-N-(6-(3-(pyridin-3-ylmethyl)ureido)hexyl)methanesulfonamide,
N-(benzyloxy)-N-(6-(3-(pyridin-4-yl)thioureido)hexyl)methanesulfonamide,
(E)-N-(benzyloxy)methylsulfonamido)hexyl)-3-(pyridin-3-yl)acrylamide,
N-(benzyloxy)-N-(6-(3-(pyridin-4-yl)ureido)hexyl)methanesulfonamide,
N-(benzyloxy)-N-(6-(2-cyano-3-(pyridin-4-yl)guanidino)heptyl)methanesulfonamide,
N-(benzyloxy)-N-(6-(3,4-dioxo-2-(pyridin-4-ylamino)cyclobut-1-enylamino)heptyl)methanesulfonamide,
N-(benzyloxy)-N-(6-(3-(pyridin-3-ylmethyl)ureido)heptyl)methanesulfonamide,
N-(benzyloxy)-N-(6-(3-(pyridin-4-yl)thioureido)heptyl)methanesulfonamide,
(E)-N-(benzyloxy)methylsulfonamido)heptyl)-3-(pyridin-3-yl)acrylamide,
N-(benzyloxy)-N-(6-(3-(pyridin-4-yl)ureido)heptyl)methanesulfonamide,
N-(8-(2-cyano-3-pyridin-4-yl)guanidino)octyl)-N-(4-fluorobenzyloxy)methanesulfonamide,
N-(8-(3,4-dioxo-2-(pyridin-4-ylamino)cyclobute-1-enylamino)octyl)-N-(4-fluorobenzyloxy)methansulfonamide,
N-(4-fluorobenzyloxy)-N-(8-(3-pyridin-3-ylmethyl)ureido)octyl)methansulfonamide,
N-(8-(N-(4-fluorobenzyloxy)methylsulfonamido)octyl-3-(pyridin-3-yl)acrylamide,
N-(4-fluorobenzyloxy)-N-(8-(3-(pyridin-4-yl)ureido)octyl)methanesulfonamide, and
N-(4-fluorobenzyloxy)-N-(8-(3-(pyridin-4-yl)thioureido)octyl)methanesulfonamide,

### General Synthesis

The compounds of the present invention can be synthesized using the methods outlined below, together with methods known in the art of organic synthetic organic chemistry, or variations thereof as appreciated by those skilled in the art. Preferred methods include, but are not limited to, those described below.

The novel compounds of formula (I) may be prepared using the reactions and techniques described in this section. The reactions are performed in solvents appropriate to the reagents and materials employed and suitable for the transformations being effected. Also, in the synthetic methods described below, it is to be understood that all proposed reaction conditions, including choice of solvent, reaction atmosphere, reaction temperature duration of experiment and work-up procedures, are chosen to be conditions of standard for that reaction, which should be readily recognized by one skilled in the art. It is understood by one skilled in the art of organic synthesis that the functionality present on various portions of the educt molecule must be compatible with the reagents and reactions proposed. Not all molecules of formula (I) falling into a given class may be compatible with some of the reaction conditions required in some of the methods described. Such restrictions to the substituents which are compatible with the reaction conditions will be readily apparent to one skilled in the art and alternative methods can be used.

Compounds (I) according to the present invention which are cyanoguanidines (Ia) can be prepared from dimethyl cyanocarbonimidodithioate and an amine of general formula (II) followed by reaction with an amine of general formula (IV). Diphenyl cyanocarbonimidate may be employed instead of dimethyl cyanocarbonimidodithioate.

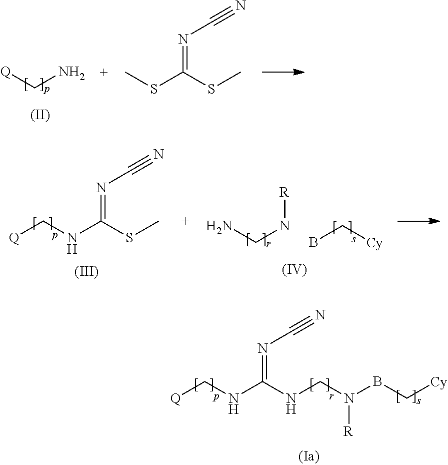

Compounds (I) of the present invention which are thioureas (Ib) can be prepared by reaction of isothiocyanates of general formula (V), which are either commercially available or prepared by literature procedures (e.g. by reaction of the corresponding amine and di(2-pyridyl)thionocarbonate: S. Kim, K. Y. Yi: Tet. Lett. (1985) 26, 1661) and an amine of general formula (IV).

Compounds (I) of the present invention which are cyanoguanidines (Ia) can also be prepared from thioureas (Ib) as described in the literature (e.g. S. K. Hamilton et al.: Org. Lett. (2005) 7 (12)2429-2431; Bioorg. Med. Chem. Lett. (1997) (24) 3095-3100; J. K. Lynch et al.: Synth. Comm. (2005) 35(1) 1-7), e.g. by reaction with cyanamide, dicyclohexylcarbodiimide and triethylamine, by reaction with EDC, cyanamide, 2,6-lutidine and titanium isopropoxide or by methylation and subsequent reaction with sodium hydrogencyanamide.

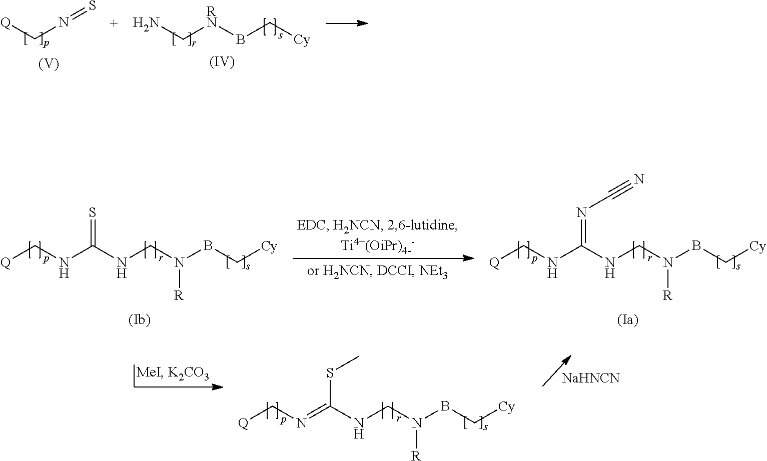

Compounds (I) according to the present invention which are ureas (Ic) can be prepared in several ways, e.g. by reaction of amines of general formula (II) with 1,1′-carbonyldiimidazole (CDI) or 4-nitrophenyl chloroformate followed by reaction with amines of general formula (IV).

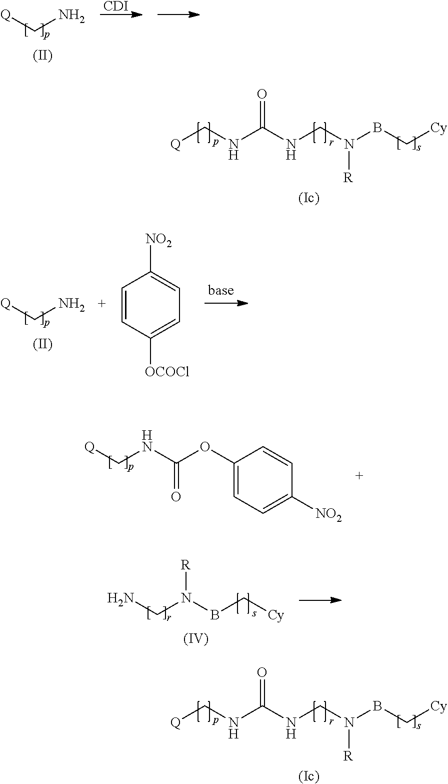

Compounds of general formula (I) which are cyclobut-3-ene-1,2-diones (squaric acids) (Id) can be prepared from reaction of amines (II) and 3,4-diethoxycyclobut-3-ene-1,2-dione to yield intermediates of general formula (VI) followed by reaction with amines (IV).

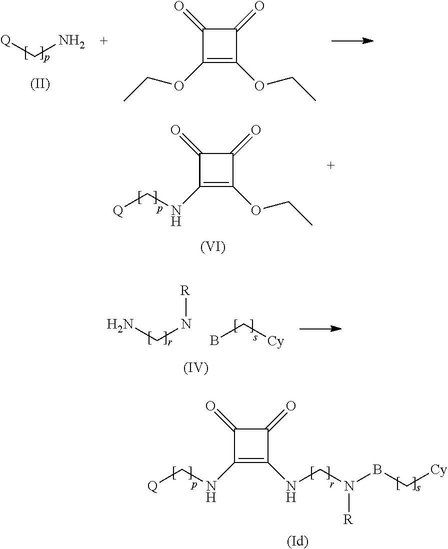

Compounds of general formula (I) which are acrylamides (Ie) can be prepared by coupling of acids of general formula (XXI) with amines of general formula (IV) using a peptide coupling reagent (e.g. EDC or HATU).

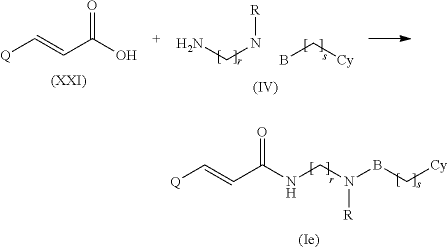

Amines of general formula (IV) containing an amine moiety at the other end (IVa) can be prepared by alkylation of amines of general formula (VII) using alkylbromides of general formula (VIII) (protecting group (Pg) e.g. phtalimido or Boc) followed by deprotection (by e.g. hydrazine hydrate or HCl, respectively).

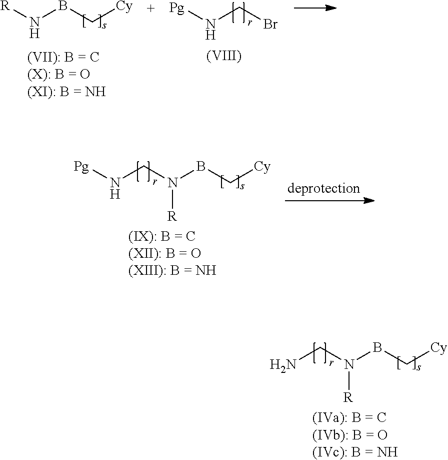

In a similar manner, amines of general formula (IV) which are hydroxylamines (IVb) or hydrazines (IVc), respectively, can be prepared by alkylation of hydroxylamines (X) or hydrazines (XI) using alkylbromides of general structure (VIII) as described in the literature (Can. J. Chem (2000) (78) 542-545) followed by deprotection.

The alkylbromides (VIII) are commercially available or can be prepared e.g. from dibromoalkyls by reaction with phthalimide or by reaction of potassium phtalimide with an aminoalcohol followed by bromination according to literature procedures (Hou et al: JOC (2004) (69) 6094-6099).

Amines in which R is hydrogen (VIIa) or alkyl (VIIb) are either commercially available or can be prepared by reductive amination of amines with aldehydes or ketones.

Hydroxylamines in which R is hydrogen (Xa) or alkyl (Xb) are either commercially available or can be prepared from N-hydroxyphtalimide (or alternatively tert-butylhydroxycarbamate) by alkylation with a halogenide and a base (e.g. DBU) or a Mitsunobu reaction with an alcohol (using e.g. DEAD), followed by deprotection with hydrazine or methylhydrazine, resulting in hydroxylamines (Xa). The resulting hydroxylamine (Xa) may be submitted to reductive amination with an aldehyde or ketone followed by reduction with e.g. sodium cyanoborohydride as described in the literature (e.g. B. J. Mavunkel et al.: Eur. J. Med. Chem. (1994) 29, 659-666; T. Ishikawa et. al.: J. Antibiotics (2000), 53 (10), 1071-1085; J. Ishwara Bhat et al.: J. Chem. Soc., Perkin Trans. 2 (2000), 1435-1446) to yield hydroxylamines (Xb). Alternatively, alkylation of the hydroxylamine (Xa) can be achieved by a Mitsunobu reaction or alkylation after protection with e.g. 2-nitrophenylsulfonylchloride and subsequent removal of the protecting group (using e.g. thiophenol and cesium carbonate).

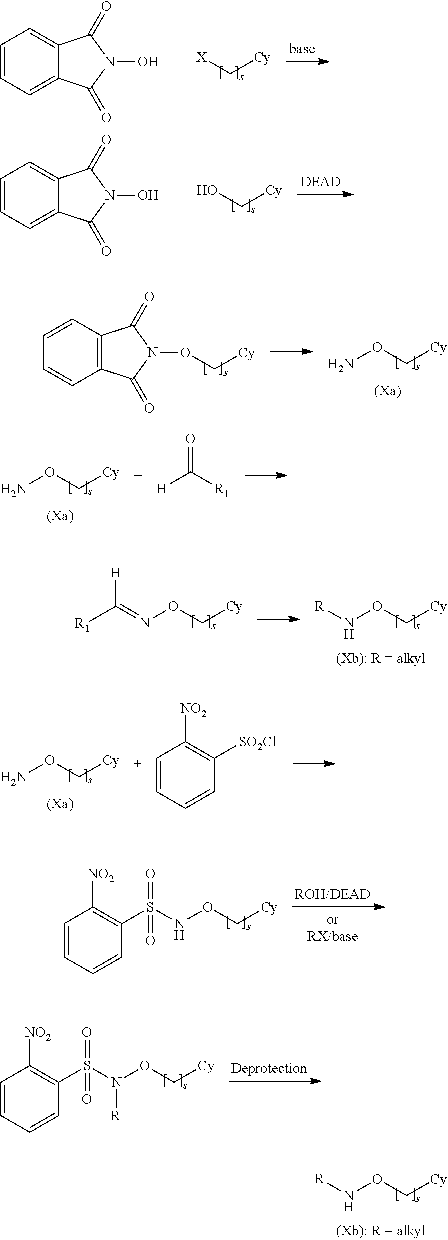

Hydrazines (XIa: R=H) or (XIb: R=alkyl) are either commercially available or can—in the case where R is H—be prepared from hydrazine hydrate by alkylation in the presence of a base according to literature procedures (e.g. D. J. Drain et al.: J. Med. Chem. (1963) δ 63-9; G. B. Marini-Bettolo et al.: Rend. Ist. Super. Sanita (1960) 23 1110-27). Hydrazines (Ib) can be obtained from monosubstituted hydrazines (XIa) by reaction with an aldehyde or ketone followed by reduction with e.g. hydrogen, LiAlH₄, or borane according to literature procedures (e.g. H. Dorn et al.: Zeitschrift für Chemie (1972) 12(4) 129-30; R. L. Hinman: JACS (1957) 79 414-417; J. A. Blair: JCS (Section) C: Organic (1970) (12) 1714-17) or alternatively by Boc-protection of hydrazine hydrate, alkylation with an alkylhalogenide in the presence of sodium hydride, followed by a second alkylation with another alkylhalogenide in the presence of sodium hydride and finally removal of the Boc-protecting groups (L. Ling et al.: Bioorg. Med. Chem. Lett. (2001) (11) 2715-2717).

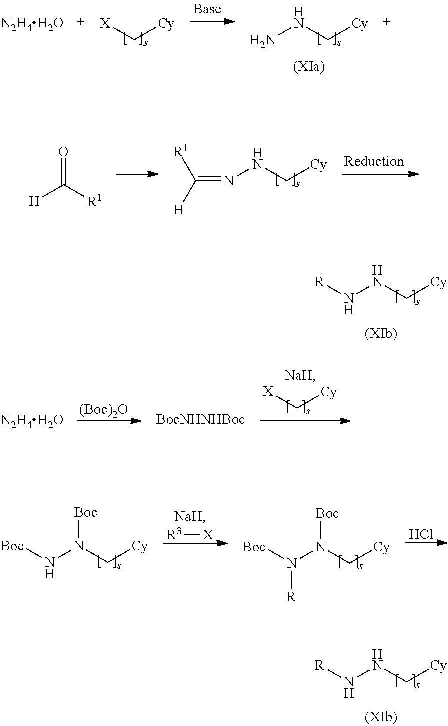

Amines of general formula (IV) which are sulfonamides (IVd), N-alkoxy or N-aryloxy sulfonamides (IVe), or N′-alkyl or N′-arylalkysulfonohydrazides (IVf) may be prepared by alkylation of sulfonamides of general formula (XIV) using alkylbromides of general formula (VIII), e.g. by treatment with Cs₂CO₃ and NaI, followed by deprotection. The sulfonamides of general formula (XIV) can be prepared by reaction of sulfonyl chlorides and amines, hydroxylamines or hydrazines, respectively.

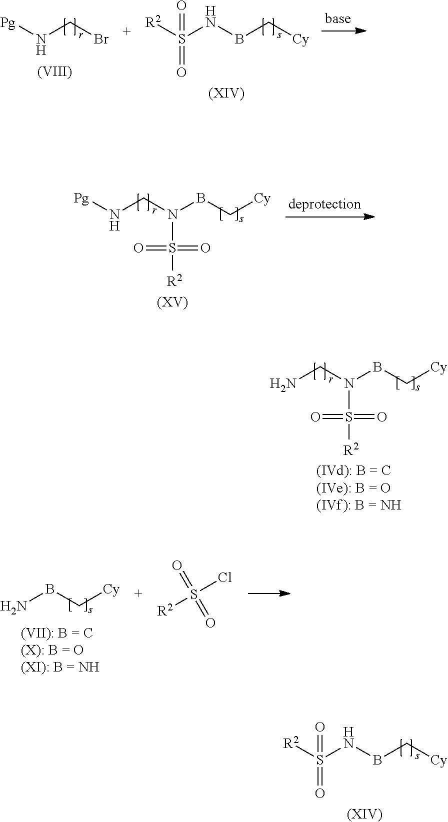

Amines of general formula (IV) which are amides (IVg) can be prepared by conversion of the mono-protected amine (XVI) to an amide by conventional amide coupling conditions (e.g. by using an acid chloride, or EDC, HOBt and NMM or TBTU and DIEA). The resulting amide is subsequently allowed to react with an alkyl bromide using e.g. Na, NaH or KOH as a base, or by a milder method using solvent-free conditions as described in the literature (e.g. Bogdal, Molecules, 4, 1999, 333-337), followed by deprotection.

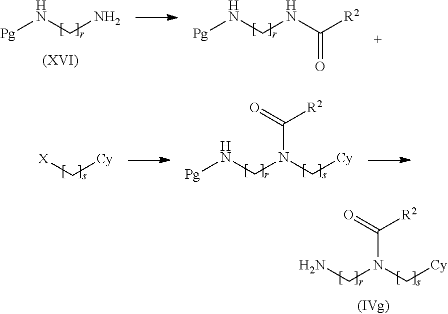

Amines of general formula (IV) which are N-alkoxy or N-phenoxy amides (IVh) or N′-alkyl or N′-arylalkyhydrazides (IVi) can be prepared from protected amino alcohols of general structure (XVII) by oxidation to aldehydes (XVIII), followed by reaction with hydroxylamines (X) or hydrazines (XI) and reduction with e.g. NaBH₄CN and HCl to yield intermediates (IXX), which can subsequently be coupled with acids using a peptide coupling reagent (e.g. EDC or HATU) followed by deprotection.

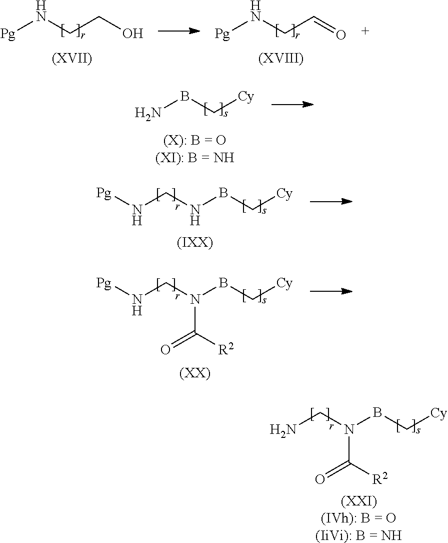

Amines of general formula (IV) can also be obtained by protection of amines (VII), hydroxylamines (X) or hydrazines (XI) with e.g. 2-nitrophenylsulfonylchloride followed by alkylation with alkyl bromides (VIII), and subsequent removal of the 2-nitrophenylsulfonyl group (using e.g. thiophenol and cesium carbonate) followed by derivatization with the appropriate reagent.

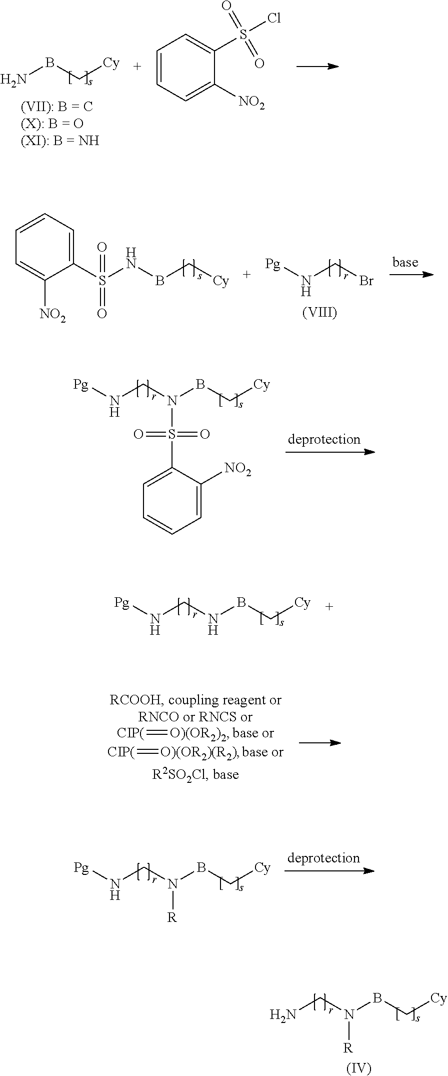

### Medical Uses

The compounds of the invention is believed to be particularly useful for down-regulating NAD via inhibition of NAMPRT, and such compounds are therefore particularly useful for treating diseases in which activation of NF-κB is implicated. Such methods are useful in the treatment of a variety of diseases including inflammatory and tissue repair disorders; particularly rheumatoid arthritis, inflammatory bowel disease, asthma and COPD (chronic obstructive pulmonary disease), osteoarthritis, osteoporosis and fibrotic diseases; dermatosis, including psoriasis, atopic dermatitis and ultra-violet induced skin damage; autoimmune diseases including systemic lupus erythematosis, multiple sclerosis, psoriatic arthritis, ankylosing spondylitis, tissue and organ rejection, Alzheimer's disease, stroke, athersclerosis, restenosis, diabetes, glomerulonephritis, cancer, particularly wherein the cancer is selected from breast, prostate, lung, colon, cervix, ovary, skin, CNS, bladder, pancreas, leukaemia, lymphoma or Hodgkin's disease, cachexia, inflammation associated with infection and certain viral infections, including Acquired Immune Deficiency Syndrome (AIDS), adult respiratory distress syndrome, ataxia telengiectasia.

Hence, the present invention provides a compound of the formula (I) for use as a medicament; more particular for use as a medicament for the treatment of a disease or a condition caused by an elevated level of nicotinamide phosphoribosyltransferase (NAMPRT), especially for the treatment of the above-mentioned diseases and conditions. Moreover, the invention also provides a method of inhibiting the enzymatic activity of nicotinamide phosphoribosyltransferase (NAMPRT) in a mammal, said method comprising the step of administering to said mammal a pharmaceutically relevant amount of a compound of the general formula (I).

Further, the invention provides a method of treating a disease or condition (in particular the diseases and conditions mentioned above) caused by an elevated level of nicotinamide phosphoribosyltransferase (NAMPRT) in a mammal, said method comprising the step of administering to said mammal a pharmaceutically relevant amount of a compound of the general formula (I).

In such methods, the compound may be administered in combination with a DNA damaging agent.

### Formulation of Pharmaceutical Compositions

The compounds of the general formula (I) are suitably formulated in a pharmaceutical composition so as to suit the desirable route of administration.

The administration route of the compounds may be any suitable route which leads to a concentration in the blood or tissue corresponding to a therapeutic effective concentration. Thus, e.g., the following administration routes may be applicable although the invention is not limited thereto: the oral route, the parenteral route, the cutaneous route, the nasal route, the rectal route, the vaginal route and the ocular route. It should be clear to a person skilled in the art that the administration route is dependent on the particular compound in question; particularly the choice of administration route depends on the physico-chemical properties of the compound together with the age and weight of the patient and on the particular disease or condition and the severity of the same.

The compounds may be contained in any appropriate amount in a pharmaceutical composition, and are generally contained in an amount of about 1-95%, e.g. 1-10%, by weight of the total weight of the composition. The composition may be presented in a dosage form which is suitable for the oral, parenteral, rectal, cutaneous, nasal, vaginal and/or ocular administration route. Thus, the composition may be in form of, e.g., tablets, capsules, pills, powders, granulates, suspensions, emulsions, solutions, gels including hydrogels, pastes, ointments, creams, plasters, drenches, delivery devices, suppositories, enemas, injectables, implants, sprays, aerosols and in other suitable form.

The pharmaceutical compositions may be formulated according to conventional pharmaceutical practice, see, e.g., “Remington's Pharmaceutical Sciences” and “Encyclopedia of Pharmaceutical Technology”, edited by Swarbrick, J. & J. C. Boylan, Marcel Dekker, Inc., New York, 1988. Typically, the compounds defined herein are formulated with (at least) a pharmaceutically acceptable carrier or excipient. Pharmaceutically acceptable carriers or excipients are those known by the person skilled in the art. Formation of suitable salts of the compounds of the Formula (I) will also be evident in view of the before-mentioned.

Thus, the present invention provides in a further aspect a pharmaceutical composition comprising a compound of the general Formula (I) in combination with a pharmaceutically acceptable carrier.

Pharmaceutical compositions according to the present invention may be formulated to release the active compound substantially immediately upon administration or at any substantially predetermined time or time period after administration. The latter type of compositions is generally known as controlled release formulations.

In the present context, the term “controlled release formulation” embraces i) formulations which create a substantially constant concentration of the drug within the body over an extended period of time, ii) formulations which after a predetermined lag time create a substantially constant concentration of the drug within the body over an extended period of time, iii) formulations which sustain drug action during a predetermined time period by maintaining a relatively, constant, effective drug level in the body with concomitant minimization of undesirable side effects associated with fluctuations in the plasma level of the active drug substance (saw-tooth kinetic pattern), iv) formulations which attempt to localize drug action by, e.g., spatial placement of a controlled release composition adjacent to or in the diseased tissue or organ, v) formulations which attempt to target drug action by using carriers or chemical derivatives to deliver the drug to a particular target cell type.

Controlled release formulations may also be denoted “sustained release”, “prolonged release”, “programmed release”, “time release”, “rate-controlled” and/or “targeted release” formulations.

Controlled release pharmaceutical compositions may be presented in any suitable dosage forms, especially in dosage forms intended for oral, parenteral, cutaneous nasal, rectal, vaginal and/or ocular administration. Examples include single or multiple unit tablet or capsule compositions, oil solutions, suspensions, emulsions, microcapsules, microspheres, nanoparticles, liposomes, delivery devices such as those intended for oral, parenteral, cutaneous, nasal, vaginal or ocular use.

Preparation of solid dosage forms for oral use, controlled release oral dosage forms, fluid liquid compositions, parenteral compositions, controlled release parenteral compositions, rectal compositions, nasal compositions, percutaneous and topical compositions, controlled release percutaneous and topical compositions, and compositions for administration to the eye will be well-known to those skilled in the art of pharmaceutical formulation. Specific formulations can be found in “Remington's Pharmaceutical Sciences”.

Capsules, tablets and pills etc. may contain for example the following compounds: microcrystalline cellulose, gum or gelatin as binders; starch or lactose as excipients; stearates as lubricants; various sweetening or flavouring agents. For capsules the dosage unit may contain a liquid carrier like fatty oils. Likewise coatings of sugar or enteric agents may be part of the dosage unit. The pharmaceutical compositions may also be emulsions of the compound(s) and a lipid forming a micellular emulsion.

For parenteral, subcutaneous, intradermal or topical administration the pharmaceutical composition may include a sterile diluent, buffers, regulators of tonicity and antibacterials. The active compound may be prepared with carriers that protect against degradation or immediate elimination from the body, including implants or microcapsules with controlled release properties. For intravenous administration the preferred carriers are physiological saline or phosphate buffered saline.

### Dosages

In one embodiment, the pharmaceutical composition is in unit dosage form. In such embodiments, each unit dosage form typically comprises 0.1-500 mg, such as 0.1-200 mg, e.g. 0.1-100 mg, of the compound.

More generally, the compound are preferably administered in an amount of about 0.1-250 mg per kg body weight per day, such as about 0.5-100 mg per kg body weight per day.

For compositions adapted for oral administration for systemic use, the dosage is normally 0.5 mg to 1 g per dose administered 1-4 times daily for 1 week to 12 months depending on the disease to be treated.

The dosage for oral administration of the composition in order to prevent diseases or conditions is normally 1 mg to 100 mg per kg body weight per day. The dosage may be administered once or twice daily for a period starting 1 week before the exposure to the disease until 4 weeks after the exposure.

For compositions adapted for rectal use for preventing diseases, a somewhat higher amount of the compound is usually preferred, i.e. from approximately 1 mg to 100 mg per kg body weight per day.

For parenteral administration, a dose of about 0.1 mg to about 100 mg per kg body weight per day is convenient. For intravenous administration, a dose of about 0.1 mg to about 20 mg per kg body weight per day administered for 1 day to 3 months is convenient. For intraarticular administration, a dose of about 0.1 mg to about 50 mg per kg body weight per day is usually preferable. For parenteral administration in general, a solution in an aqueous medium of 0.5-2% or more of the active ingredients may be employed.

For topical administration on the skin, a dose of about 1 mg to about 5 g administered 1-10 times daily for 1 week to 12 months is usually preferable.

### EXPERIMENTALS

### General Procedures, Preparations and Examples

For nuclear magnetic resonance ¹H NMR spectra (300 MHz) and ¹³C NMR (75.6) chemical shift values (δ) (in ppm) are quoted, unless otherwise specified, for deuteriochloroform solutions relative to tetramethylsilane (δ=0.0) or chloroform (δ=7.25) or deuteriochloroform (δ=76.81 for ¹³C NMR) standards. ¹H NMR spectra in CD₃OD were referenced to CHD₂OD: 3.33 ppm; CDCl₃ to CHCl₃: 7.26 ppm, DMSO-d₆ to CHD₂SOCD₃: 2.50 ppm The value of a multiplet, either defined (dublet (d), triplet (t), double dublet (dd), double triplet (dt), quartet (q)) or not (m) at the approximate mid point is given unless a range is quoted. (bs) indicates a broad singlet. NMR spectra were recorded at 300 MHz on a Bruker Avance 300 system.

MS was performed using an LC-MS using a Bruker Esquire 3000+ ESI Iontrap with an Agilent 1200 HPLC-system. The organic solvents used were anhydrous.

S-Methyl N-cyano-N′-4-pyridylisothiourea was prepared as described in Bioorg. Med. Chem. Lett. (1997) 7 (24), 3095-3100.

3-Ethoxy-4-(pyridin-4-ylamino)cyclobut-3-ene-2,3-dione and 3-ethoxy-4-(pyridin-3-ylamino)cyclobut-3-ene-2,3-dione were prepared as described in J. Med. Chem. (2000) 43 1187-1202.

The following abbreviations have been used throughout:
CDI 1,1′-carbonyldiimidazole
DCCI dicyclohexylcarbodiimide
DCM dichloromethane
DCE 1,2-dichloroethane
DIEA diisopropylehylamine
DMF N,N-dimthylformamide
DMAP N,N dimethylaminopryridine
DPT di(2-pyridyl) thionocarbonate
EDC N-(dimethylaminopropyl)-N′-ethylcarbodiimide hydrochloride
EtOAc ethyl acetate
HATU O-(7-Azabenzotriazol-1-yl)-N,N,N′,N′-tetramethyluronium hexafluorophosphate
HOBt 1-hydroxybenzotriazole
MS mass spectroscopy
NMM N-methylmorpholine
NMR nuclear magnetic resonance
rt room temperature
TBTU O-(Benzotriazol-1-yl)-N,N,N′,N′-tetramethyluronium tetrafluoroborate
THF tetrahydrofuran
TLC thin layer chromatography
   General Procedure 1: Reaction of Potassium Phthalimide with Bromoalkanols.

A solution of potassium phthalimide (1 eq) and a bromoalkanol (1 eq) in dry DMF (1 mL/mmol) was heated at 158° C. overnight. The reaction mixture was cooled to rt, concentrated to dryness and the residue was dissolved in EtOAc. The organic phase was washed with H₂O (2 times), brine, dried (MgSO₄) and concentrated to yield a pthalimide protected aminoalkanol.

### General Procedure 2: Bromination of Phtalimide Protected Aminoalkanols to Give Alkylbromides (VIII).

To a solution of a phtalimide protected aminalkanol (1 eq) in CH₃CN (3 mL/mmol) was added PPh₃ (1 eq) and CBr₄ (1 eq) and the mixture was stirred at rt for 2½ h. The reaction mixture was concentrated and purified by chromatography (mixtures of petroleum ether and EtOAc) to afford the corresponding alkylbromide (VIII).

### General Procedure 3: Alkylation of Amines of General Formula (VII) Using Alkylbromides of General Formula (VIII) to Give Protected Amines of General Formula (IX).

A solution of an alkylbromide of general formula (VIII) (1 eq) in dry DMF (0.5 mL/mmol) was added to a flask suited for microwave heating. The amine (VII) (1 eq) was dissolved in DMF and added to the reaction flask and then K₂CO₃ (3 eq) was added. The reaction mixture was heated in a microwave oven at 70° C. for 3 h. The reaction was quenched with water, extracted with EtOAc and the organic phase concentrated to dryness, purified by chromatography using appropriate mixture of MeOH/CHCl₃/NH₃ to afford protected amines of general formula (IX).

### General Procedure 4: Deprotection of Phtalimide Protected Amines.

A solution of a pthalimide protected amine (6 mL/mmol) was added to a flask suited for microwave heating. Hydrazine hydrate (5 eq) was added and the mixture heated in microwave oven at 130° C. for 20 min. After cooling the white precipitate formed was removed by filtration. The filtercake was washed with EtOH and the filtrate was concentrated. The residue was purified by chromatography using appropriate mixture of MeOH/CHCl₃/NH₃ to afford deprotected amines.

General Procedure 5: Preparation of Cyanoguanidines of General Formula (Ia) by Reaction of Intermediates of General Formula (III) with Amines of General Formula (IV).

Intermediate of general formula (III) (1.0 eq.) was dissolved in pyridine, amine of general formula (XXII) (1.05 eq.), triethylamine (1.1 eq.) and polystyrene-supported DMAP (catalytic amount) were added and the mixture heated with stirring at 80° C. overnight or until consumption of starting material (III). The reaction mixture was concentrated twice with toluene, the residue purified by chromatography (chloroform:methanol:NH₃ (25% aq.) 98:2:0.2 to 95:5:1) to afford cyanoguanidines of general formula (Ia).

General Procedure 6: Reaction of Amines of General Formula (II) with 4-Nitrophenyl Chloroformate Followed by Reaction with Amines of General Formula (IV) to Yield Ureas of General Formula (Ic).

Amine of general formula (II) (1.0 eq.) was dissolved in EtOAc, DIEA (1.2 eq.) was added, the mixture was cooled on an icebath and 4-nitrophenyl chloroformate (1.1 eq.) was added with stirring. After 4 h (or consumption of the amine (II)) the reaction mixture was washed successively with 5% Na₂CO₃ (twice), H₂O, brine, dried over Mg₂SO₄, filtered and concentrated. The resulting 4-nitrophenyl carbamate (1.0 eq.) was dissolved in DMF, amine of general formula (IV) (1.0 eq.) was added followed by HOBt (2.0 eq) and DIEA (0.5 eq.) and heated at 40° C. overnight. The mixture was concentrated and purified by chromatography (chloroform:methanol:NH₃ (25% aq.) 98:2:0.2 to 96:4:0.4) to afford urea of general formula (Ic).

General Procedure 7: Reaction of Amines of General Formula (IV) with 3-ethoxy-cyclobut-3-ene-1,2-diones of General Formula (VI) to Yield cyclobut-3-ene-1,2-diones of General Formula (Id).

Amine of general formula (IV) (1.02 eq) and 3-ethoxy-cyclobut-3-ene-1,2-dione of general formula (VI) (1.0 eq) were dissolved in acetonitrile (if the amine is a salt, 1.0 eq. of triethylamine is added) and stirred at rt until consumption of starting material as judged by TLC. The product was either purified by crystallization or chromatography (chloroform:methanol:NH₃ (25% aq.) 98:2:0.2 to 95:5:1) to afford compounds of general formula (Id).

### General Procedure 8: Preparation of Sulfonamides of General Formula (XIV).

The sulfonylchloride (1.02 eq.) was added in small portions to a solution of amine (VII), hydroxylamine (X) or hydrazine (XI) (1.0 eq.) and triethyamine or N-methylmorpholine (1.1 eq., or 2.2 eq. if the hydroxylamine, amine or hydrazine is a salt) in DCM at 0° C. with stirring. The mixture was gradually allowed to reach rt, stirred overnight, concentrated, purified by chromatography (mixtures of MeOH/CHCl₃/NH₃ or mixtures of petroleum ether and EtOAc) to yield intermediates of general formula (XIV).

### General Procedure 9: Alkylation of Sulfonamides of General Formula (XIV) Using Alkylbromides of General Formula (VIII) to Afford Compounds of General Formula (XV).

Cs₂CO₃ (2 eq) and NaI (catalytic amount) were added to a solution of a sulfonamide of general formula (XIV) in dry DMF (4 mL/mmol) and stirred at 50° C. for 1 h. Alkylbromide of general formula (VIII) (1 eq) was added and the mixture stirred at 50° C. overnight. The reaction mixture was concentrated, diluted with EtOAc and washed with H₂O. The water phase was extracted with EtOAc and the collected organic phases concentrated. The residue was purified by chromatography (mixtures of MeOH/CHCl₃/NH₃ or mixtures of petroleum ether and EtOAc) to yield intermediates of general formula (XV).

General Procedure 10: Coupling of Acids of General Formula (XXI) with Amines of General Formula (IV) to Afford Compounds of General Formula (Ie).

Acid of general formula (XXI) (1 eq.) and amine of general formula (IV) were dissolved in DMF. HOBt (1 eq.), NMM (1 eq.) and EDC (1.3 eq.) were added with stirring and the reaction mixture was stirred at rt overnight. The solvent was evaporated in vacuo and the residue was purified by chromatography (mixtures of MeOH/CHCl₃/NH₃ (25% aq.)) to yield compounds of general formula (Ie).

General Procedure 11: Reaction of Amines of General Formula (II) with CDI Followed by Reaction with Amines of General Formula (IV) to Yield Ureas of General Formula (Ic).

To a solution of CDI (1.1 eq.) in THF was added amine of general formula (II) (1.0 eq.) and the mixture was stirred at rt overnight. To the reaction mixture amine of general formula (IV) (1.0 eq.) was added and the reaction was stirred at rt overnight. The solvent was evaporated in vacuo and the residue was purified by chromatography (chloroform:methanol:NH₃ (25% aq.) 96:4:0.4 or with MeCN—H₂O—AcOH 3:1:1) to yield urea of general formula (Ic).

The oxalic acid salt of urea of general formula (Ic) may be obtained by dissolving compound of general formula (Ic) (1 eq.) in MeCN and adding a solution of oxalic acid (2 eq.) in MeCN. The precipitate was filtered and dried to give the oxalic acid salt of urea of general formula (Ic).

The HCl-salt of urea of general formula (Ic) may be obtained by dissolving compound of general formula (Ic) (1 eq) in 1N HCl/MeOH (2 eq.), the solvent was evaporated in vacuo, the residue was washed with DCM followed by Et₂O and dried to give the HCl-salt of urea of general formula (Ic).

General Procedure 12: Reaction of Amines of General Formula (II) with DPT Followed by Reaction with Amines of General Formula (IV) to Yield Thioureas of General Formula (Ib).

Amine of general formula (II) (1.0 eq.) was dissolved in THF, the reaction mixture was cooled on an icebath and NaH (1.1 eq.) was added with stirring. After 1 h DPT (1.0 eq.) was added and the mixture gradually allowed to reach rt. After a further 3 h (or consumption of the starting materials) the resulting isothiocyanate was either purified by chromatography (mixtures of petroleum ether and ETOAc) or used directly.

To a solution in THF of the isothiocyanate (1.0 eq.) was added amine of general formula (IV) (1.0 eq.) and DIEA (1.1 eq.) and the mixture was stirred at rt overnight, concentrated and purified by chromatography (1-5% methanol in DCM) to afford thiourea of general formula (Ib).

The oxalic acid salt of thiourea of general formula (Ib) may be obtained by dissolving compound of general formula (Ib) (1 eq.) in MeCN and adding a solution of oxalic acid (2 eq.) in MeCN. The precipitate was filtered and dried to give the oxalic acid salt of urea of general formula (Ib).

### Preparation 1

### N-(3-morpholinopropyl)cyclohexanamine (Compound 1)

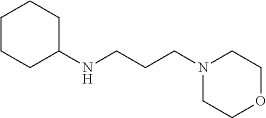

3-Morpholinopropylamine (1.46 mL, 10 mmol) and cyclohexanone (1.04 mL, 10 mmol) were dissolved in dichloroethane, sodium triacetoxyborohydride (3.18 g, 15 mmol) was added in small portions with stirring, and the mixture was stirred at room temperature overnight. 1 N NaOH was added carefully, and the mixture extracted 3 times with DCM. The collected organic phases were washed with brine, dried (MgSO₄) and concentrated to yield compound 91. ¹H-NMR (DMSO-d₆): δ 3.55 (m, 4H), 2.53 (t, 2H), 2.31 (m, 7H), 1.78 (m, 2H), 1.65 (m, 2H), 1.53 (m, 2H) 1.17 (m, 4H), 0.99 (m, 2H).

### Preparation 2

### (N-(8-hydroxyoctyl)phthalimide (Compound 2)

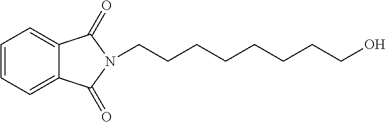

General procedure 1. Starting material: 8-bromo-1-octanol. ¹H-NMR (DMSO-d₆): δ 7.88-7.81 (m, 4H), 4.33 (t, 1H, OH), 3.55 (t, 2H), 3.38-3.32 (m, 2H), 1.59-1.55 (m, 2H), 1.40-1.36 (m, 2H), 1.25-1.23 (m, 8H).

### Preparation 3

### N-(8-bromooctyl)phthalimide (Compound 3)

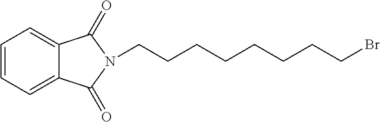

General procedure 2. Starting material: compound 2. ¹H-NMR (DMSO-d₆): δ 7.89-7.82 (m, 4H), 3.58-3.49 (m, 4H), 1.81-1.72 (m, 2H), 1.63-1.54 (m, 2H), 1.40-1.23 (m, 8H).

### Preparation 4

### N-(7-hydroxyheptyl)phthalimide (Compound 4)

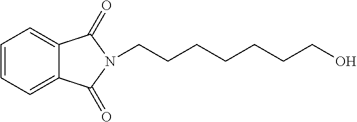

General procedure 1. Starting material: 7-bromoheptan-1-ol. ¹H-NMR (CDCl₃): δ 7.85-7.82 (m, 2H), 7.72-7.69 (m, 2H), 3.70-3.61 (m, 4H), 1.73-1.63 (m, 2H), 1.60-1.51 (m, 2H), 1.41-1.33 (m, 6H).

### Preparation 5

### N-(7-bromoheptyl)phthalimide (Compound 5)

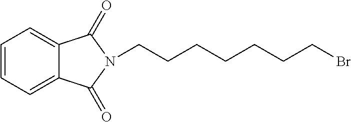

General procedure 2. Starting material: compound 4. ¹H-NMR (CDCl₃): δ 7.84 (dd, 2H), 7.70 (dd, 2H), 3.67 (t, 2H), 3.39 (t, 2H), 1.88-1.79 (m, 2H), 1.72-1.63 (m, 2H), 1.47-1.33 (m, 6H).

### Preparation 6

### 2-(8-(cyclohexyl(3-morpholinopropyl)amino)octyl)isoindoline-1,3-dione (Compound 6)

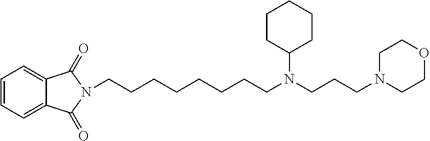

General procedure 3. Starting materials: compounds 1 and 3. ¹H-NMR (DMSO-d₆): δ 7.89-7.82 (m, 4H), 3.58-3.52 (m, 6H), 2.39-2.21 (m, 11H), 1.71-1.08 (m, 24H).

### Preparation 7

### 2-(7-(cyclohexyl(3-morpholinopropyl)amino)heptyl)isoindoline-1,3-dione (Compound 7)

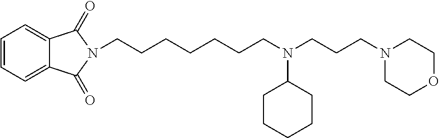

General procedure 3. Starting materials: compounds 1 and 5. ¹H-NMR (DMSO-d₆): δ 7.89-7.82 (m, 4H), 3.58-3.51 (m, 6H), 2.39-2.20 (m, 11H), 1.71-1.08 (m, 22H).

### Preparation 8

### tert-Butyl 6-(cyclohexyl(3-morpholinopropyl)amino)hexylcarbamate (Compound 8)

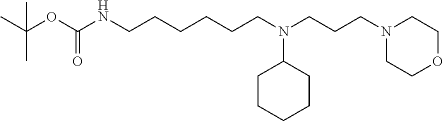

6-(Boc-amino)hexyl bromide (0.25 g, 0.80 mmol) was added to a flask suited for microwave heating. The amine 1 (0.165 g, 0.73 mmol) was dissolved in dry DMF (0.7 mL) and added to the flask together with K₂CO₃ (0.316 g, 2.28 mmol). The reaction mixture was heated in a microwave oven at 70° C. for 2 h. The mixture was quenched using water and extracted with EtOAc, the organic phase was dried (MgSO₄) and concentrated. The residue was purified by chromatography (CHCl₃:MeOH:NH₃ 94:4:1) to afford compound 8. ¹H-NMR (DMSO-d₆): δ 3.55 (t, 4H), 2.91-2.84 (m, 2H), 2.41-2.23 (m, 11H), 1.74-1.10 (m, 29H).

### Preparation 9

### N¹-cyclohexyl-N¹-(3-morpholinopropyl)octane-1,8-diamine (Compound 9)

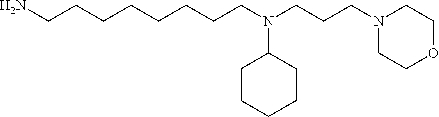

General procedure 4. Starting material: compound 6. ¹H-NMR (CDCl₃): δ 3.69 (t, 4H), 2.65 (t, 2H), 2.46-2.28 (m, 11H), 1.74-1.04 (m, 24H).

### Preparation 10

### N¹-cyclohexyl-N¹-(3-morpholinopropyl)heptane-1,7-diamine (Compound 10)

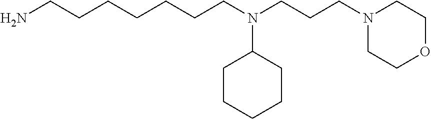

General procedure 4. Starting material: compound 7. ¹H-NMR (DMSO-d₆): δ 3.55 (t, 4H), 2.54 (t, 2H), 2.41-2.23 (m, 11H), 1.75-1.10 (m, 22H).

### Preparation 11

### N¹-cyclohexyl-N¹-(3-morpholinopropyl)hexane-1,6-diamine (Compound 11)

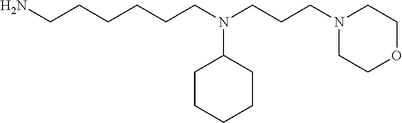

Compound 8 (0.100 g, 0.235 mmol) was dissolved in HCl in MeOH (3M, 2 mL) and stirred at rt for 4.5 h. The reaction mixture was then concentrated to dryness and the residue purified by chromatography (CHCl₃:MeOH:NH₃ 90:10:1) to afford compound 11 (not quite pure). Used for next step without further purification. ¹H-NMR (CDCl₃): δ 3.72 (t, 4H), 2.72-0.88 (m, 33H).

### Preparation 12

### N-(3-morpholinopropyl)cyclopentanesulfonamide (Compound 12)

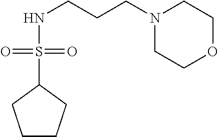

General procedure 8. Starting materials: 3-morpholinopropylamine and cyclopentyl sulfonyl chloride. ¹H-NMR (DMSO-d₆): δ 6.99 (t, 1H, NH), 3.57-3.46 (m, 5H), 2.97 (q, 2H), 2.34-2.28 (m, 6H), 1.90-1.76 (m, 4H), 1.68-1.52 (m, 6H).

### Preparation 13

### N-(3-morpholinopropyl)cyclohexanesulfonamide (Compound 13)

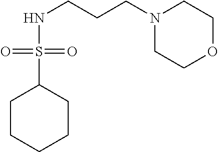

General procedure 8. Starting materials: 3-morpholinopropylamine and cyclohexyl sulfonyl chloride. ¹H-NMR (DMSO-d₆): δ 6.95 (t, 1H, NH), 3.56 (t, 4H), 2.99-2.84 (m, 3H), 2.32-2.27 (m, 6H), 2.02-1.99 (m, 2H), 1.80-1.76 (m, 2H), 1.64-1.54 (m, 3H), 1.39-1.06 (m, 5H).

### Preparation 14

### N-(7-(1,3-dioxoisoindolin-2-yl)heptyl)-N-(3-morpholinopropyl) cyclopentanesulfonamide (Compound 14)

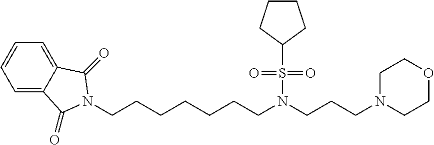

General procedure 9. Starting materials: compounds 12 and 5. ¹H-NMR (DMSO-d₆): δ 7.90-7.82 (m, 4H), 3.74-3.62 (m, 6H), 3.47-3.40 (m, 1H), 3.31-3.23 (m, 4H), 2.50 (bs, 4H), 2.41 (t, 2H), 2.03-1.36 (m, 20H).

### Preparation 15

### N-(7-(1,3-dioxoisoindolin-2-yl)heptyl)-N-(3-morpholinopropyl) cyclohexanesulfonamide (Compound 15)

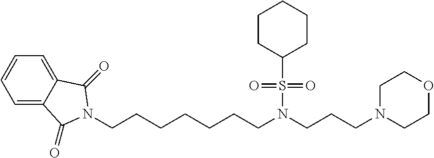

General procedure 9. Starting materials: compounds 13 and 5. ¹H-NMR (CDCl₃): δ 7.83 (dd, 2H), 7.70 (dd, 2H), 3.71-3.64 (m, 6H), 3.26-3.14 (m, 4H), 2.84 (m, 1H), 2.42 (t, 4H), 2.33 (t, 2H), 2.06 (d, 2H), 1.89-1.18 (m, 20H).

### Preparation 16

### N-(8-(1,3-dioxoisoindolin-2-yl)octyl)-N-(3-morpholinopropyl) cyclopentanesulfonamide (Compound 16)

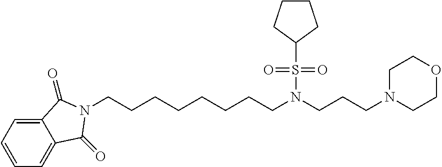

General procedure 9. Starting materials: compounds 12 and 3.

¹H-NMR (CDCl₃): δ 7.82 (dd, 2H), 7.69 (dd, 2H), 3.70-3.63 (m, 6H), 3.41 (q, 1H), 3.27-3.15 (m, 4H), 2.41 (t, 4H), 2.33 (t, 2H), 1.99-1.29 (m, 22H).

### Preparation 17

### N-(8-(1,3-dioxoisoindolin-2-yl)octyl)-N-(3-morpholinopropyl) cyclohexanesulfonamide (Compound 17)

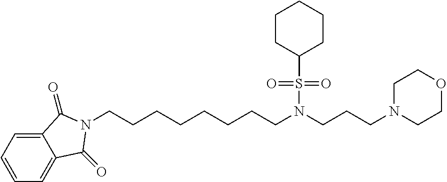

General procedure 9. Starting materials: compounds 13 and 3. ¹H-NMR (CDCl₃): δ 7.83 (dd, 2H), 7.70 (dd, 2H), 3.70-3.63 (m, 6H), 3.27-3.14 (m, 4H), 2.84 (tt, 1H), 2.41 (t, 4H), 2.33 (t, 2H), 2.06 (d, 2H), 1.89-1.44 (m, 10H), 1.31-1.13 (m, 12H).

### Preparation 18

### N-(7-aminoheptyl)-N-(3-morpholinopropyl)cyclopentane sulfonamide (Compound 18)

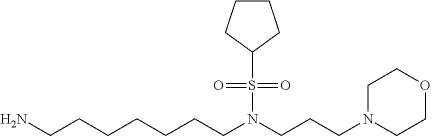

General procedure 4. Starting material: compound 14. ¹H-NMR (CDCl₃): δ 3.68 (t, 4H), 3.40 (q, 1H), 3.26-3.15 (m, 4H), 2.66 (t, 2H), 2.40 (t, 4H), 2.32 (t, 2H), 1.96-1.24 (m, 20H).

### Preparation 19

### N-(7-aminoheptyl)-N-(3-morpholinopropyl)cyclohexanesulfonamide (Compound 19)

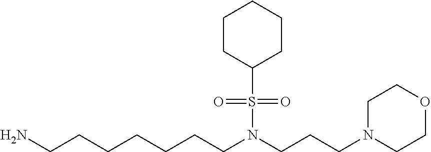

General procedure 4. Starting material: compound 15. ¹H-NMR (CDCl₃): δ 3.71 (t, 4H), 3.28-3.16 (m, 4H), 2.85 (tt, 1H), 2.68 (t, 2H), 2.43 (t, 4H), 2.34 (t, 2H), 2.07 (d, 2H), 1.91-1.19 (m, 20H).

### Preparation 20

### N-(8-aminooctyl)-N-(3-morpholinopropyl)cyclopentanesulfonamide (Compound 20)

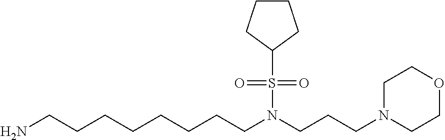

General procedure 4. Starting material: compound 16. ¹H-NMR (CD₃OD) δ: 3.71 (t, 4H), 3.70-3.58 (m, 1H), 3.32-3.22 (m, 4H), 2.64 (t, 2H), 2.49 (t, 4H), 2.41 (t, 2H), 2.02-1.37 (m, 22H).

### Preparation 21

### N-(8-aminooctyl)-N-(3-morpholinopropyl)cyclohexanesulfonamide (Compound 21)

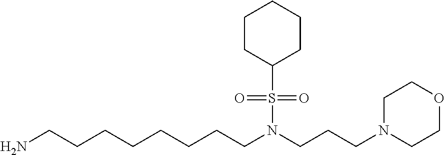

General procedure 4. Starting material: compound 17. ¹H-NMR (CDCl₃): δ 3.68 (t, 4H), 3.26-3.14 (m, 4H), 2.83 (tt, 1H), 2.66 (t, 2H), 2.40 (t, 4H), 2.32 (t, 2H), 2.05 (d, 2H), 1.88-1.17 (m, 22H).

### Preparation 22

### N-(benzyloxy)methanesulfonamide (Compound 22)

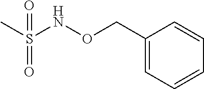

General procedure 8. Starting materials: O-benzylhydroxylamine hydrochloride and methanesulfonyl chloride. ¹H-NMR (CDCl₃): δ 7.39 (m, 5H), 6.90 (bs, 1H), 5.00 (s, 2H), 3.03 (s, 3H).

### Preparation 23

### N-(benzyloxy)-N-(8-(1,3-dioxoisoindolin-2-yl)octyl)methanesulfonamide (Compound 23)

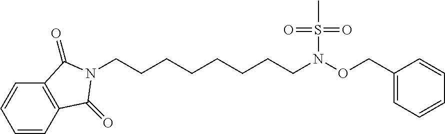

General procedure 9. Starting materials: compounds 22 and 3. ¹H-NMR (CDCl₃): δ 7.84 (dd, 2H), 7.71 (dd, 2H), 7.38 (m, 5H), 5.02 (s, 2H), 3.68 (t, 2H), 3.14 (m, 2H), 2.89 (s, 3H), 1.63 (m, 4H), 1.31 (m, 8H).

### Preparation 24

### N-(8-aminooctyl)-N-(benzyloxy)methanesulfonamide (Compound 24)

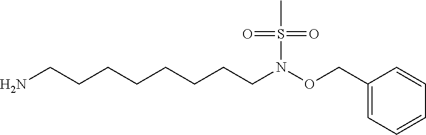

General procedure 4. Starting material: compound 23. ¹H-NMR (CD₃OD): δ 7.40 (m, 5H), 5.02 (s, 2H), 3.18 (t, 2H), 2.96 (s, 3H), 2.65 (t, 2H), 1.58 (m, 2H), 1.49 (m, 2H), 1.34 (m, 8H).

### Preparation 25

### N-(benzyloxy)propane-2-sulfonamide (Compound 25)

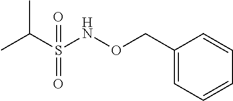

General procedure 8. Starting materials: O-benzylhydroxylamine hydrochloride and 2-propanesulfonyl chloride. ¹H-NMR (CDCl₃): δ 7.38 (m, 5H), 7.07 (bs, 1H), 4.98 (s, 2H), 3.59 (m, 1H), 1.40 (d, 6H).

### Preparation 26

### N-(benzyloxy)-N-(8-(1,3-dioxoisoindolin-2-yl)octyl)propane-2-sulfonamide (Compound 26)

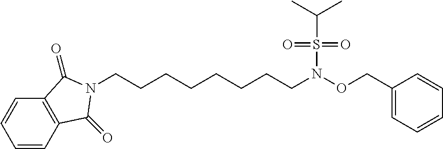

General procedure 9. Starting materials: compounds 25 and 3. ¹H-NMR (CDCl₃): δ 7.84 (dd, 2H), 7.71 (dd, 2H), 7.37 (m, 5H), 5.01 (s, 2H), 3.68 (t, 2H), 3.51 (m, 1H), 3.29 (t, 2H), 1.63 (m, 4H), 1.43 (d, 6H), 1.32 (m, 8H).

### Preparation 27

### N-(8-aminooctyl)-N-(benzyloxy)propane-2-sulfonamide (Compound 27)

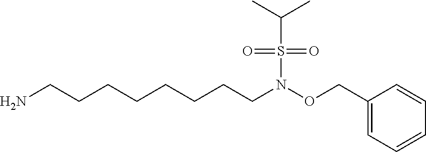

General procedure 4. Starting material: compound 26. ¹H-NMR (CD₃OD): δ 7.39 (m, 5H), 5.00 (s, 2H), 3.60 (m, 1H), 3.32 (m, 2H), 2.65 (t, 2H), 1.61 (m, 2H), 1.49 (m, 2H), 1.41 (d, 6H), 1.35 (m, 8H).

### Preparation 28

### N-(3-morpholinopropyl)methanesulfonamide (Compound 28)

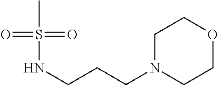

General procedure 8. Starting materials: 3-morpholinopropylamine and methanesulfonyl chloride. ¹H-NMR (CDCl₃): δ 6.59 (bs, 1H), 3.72 (t, 4H), 3.26 (t, 2H), 2.94 (s, 3H), 2.54 (t, 2H), 2.49 (m, 2H), 1.78 (m, 2H).

### Preparation 29

### N-(8-(1,3-dioxoisoindolin-2-yl)octyl)-N-(3-morpholinopropyl) methanesulfonamide (Compound 29)

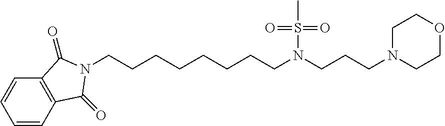

General procedure 9. Starting materials: compounds 28 and 3. ¹H-NMR (CDCl₃): δ 7.85 (m, 4H), 3.70 (m, 6H), 3.21 (m, 4H), 2.87 (s, 3H), 2.48 (m, 4H), 2.41 (t, 2H), 1.82 (m, 2H), 1.65 (m, 4H), 1.37 (m, 8H).

### Preparation 30

### N-(8-aminooctyl)-N-(3-morpholinopropyl)methanesulfonamide (Compound 30)

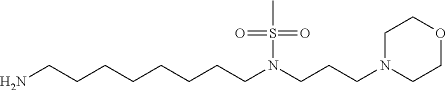

General procedure 4. Starting material: compound 29. ¹H-NMR (CD₃OD): δ 3.71 (m, 4H), 3.22 (m, 4H), 2.64 (t, 2H), 2.48 (m, 4H), 2.41 (t, 2H), 1.83 (m, 2H), 1.64 (m, 2H), 1.50 (m, 2H), 1.37 (m, 8H).

### Preparation 31

### N-(3-morpholinopropyl)benzenesulfonamide (Compound 31)

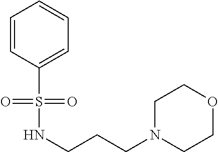

General procedure 8. Starting materials: 3-morpholinopropylamine and benzenesulfonyl chloride. ¹H-NMR (CD₃OD): δ 7.87 (m, 2H), 7.60 (m, 3H), 3.67 (t, 4H), 2.93 (t, 2H), 2.40 (t, 4H), 2.36 (t, 2H), 1.65 (m, 2H).

### Preparation 32

### N-(8-(1,3-dioxoisoindolin-2-yl)octyl)-N-(3-morpholinopropyl) benzenesulfonamide (Compound 32)

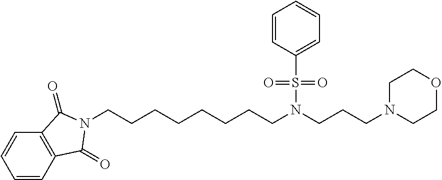

General procedure 9. Starting materials: compounds 31 and 3. ¹H-NMR (CD₃OD): δ 7.84 (m, 6H), 7.61 (m, 3H), 3.68 (m, 6H), 3.18 (m, 4H), 2.44 (t, 4H), 2.36 (t, 2H), 1.75 (m, 2H), 1.67 (m, 2H), 1.52 (m, 2H), 1.31 (m, 8H).

### Preparation 33

### N-(8-aminooctyl)-N-(3-morpholinopropyl)benzenesulfonamide (Compound 33)

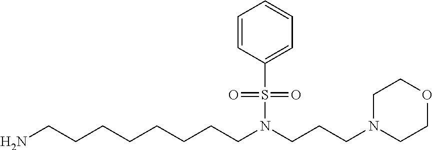

General procedure 4. Starting material: compound 32. ¹H-NMR (CD₃OD): δ 7.84 (m, 2H), 7.62 (m, 3H), 3.70 (t, 4H), 3.19 (m, 4H), 2.64 (t, 2H), 2.44 (t, 4H), 2.36 (t, 2H), 1.75 (m, 2H), 1.52 (m, 4H), 1.33 (m, 8H).

### Preparation 34

### tert-Butyl 6-(N-(3-morpholinopropyl)cyclopentanesulfonamido)hexylcarbamate (Compound 34)

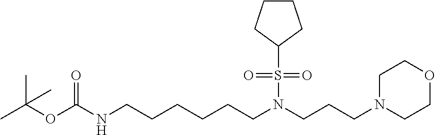

General procedure 9. Starting materials: compounds 12 and 6-(Boc-amino)hexyl bromide. ¹H-NMR (CD₃OD): δ 3.71 (m, 4H), 3.64 (m, 1H), 3.25 (m, 2H), 3.05 (m, 4H), 2.48 (m, 4H), 2.40 (t, 2H), 2.05-1.25 (m, 27H).

### Preparation 35

### N-(6-Aminohexyl)-N-(3-morpholinopropyl)cyclopentanesulfonamide (Compound 35)

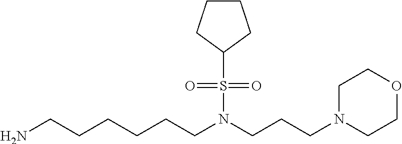

Compound 34 (0.515 g, 1.08 mmol) was dissolved MeOH (2 mL) and HCl in MeOH (3M, 3 mL) was added with stirring. After 1 h the reaction mixture was concentrated to dryness and the residue purified by chromatography (CHCl₃:MeOH:NH₃ 90:10:1) to afford compound 35. ¹H-NMR (CD₃OD) δ: 3.76 (m, 4H), 3.64 (m, 1H), 3.31 (m, 4H), 2.94 (t, 2H), 2.66 (bs, 4H), 2.57 (t, 2H), 2.1-1.3 (m, 18H).

### Preparation 36

### tert-Butyl 6-(N-(3-morpholinopropyl)cyclohexanesulfonamido)hexylcarbamate (Compound 36)

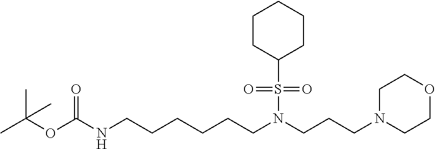

General procedure 9. Starting materials: compounds 13 and 6-(Boc-amino)hexyl bromide. ¹H-NMR (CD₃OD): δ 3.72 (t, 4H), 3.27 (m, 4H), 3.05 (m, 3H), 2.49 (m, 4H), 2.40 (t, 2H), 2.09 (m, 2H), 1.95-1.15 (m, 27H).

### Preparation 37

### N-(6-Aminohexyl)-N-(3-morpholinopropyl)cyclohexanesulfonamide (Compound 37)

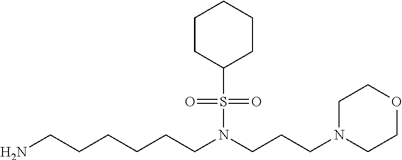

Compound 36 (0.47 g, 0.91 mmol) was dissolved MeOH (2 mL) and HCl in MeOH (3M, 3 mL) was added with stirring. After 1 h the reaction mixture was concentrated to dryness and the residue purified by chromatography (CHCl₃:MeOH:NH₃ 90:10:1) to afford compound 37. ¹H-NMR (CD₃OD): δ 3.72 (m, 4H), 3.28 (m, 4H), 3.05 (m, 1H), 2.67 (t, 2H), 2.48 (m, 4H), 2.40 (t, 2H), 2.08 (m, 2H), 1.95-1.1 (m, 18H).

### Preparation 38

### 1-Phenyl-N-(tetrahydro-2H-pyran-2-yloxy)methanesulfonamide (Compound 38)

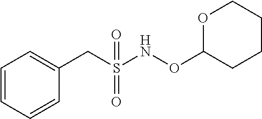

General procedure 8. Starting materials: 3-morpholinopropylamine and benzenesulfonyl chloride. ¹H-NMR (CDCl₃): δ 7.42 (m, 5H), 7.01 (s, 1H), 5.12 (m, 1H), 4.58 (d, 1H), 4.36 (d, 1H), 3.86 (m, 1H), 3.62 (m, 1H), 1.9-1.5 (m, 6H).

### Preparation 39

### N-(7-(1,3-dioxoisoindolin-2-yl)heptyl)-1-phenyl-N-(tetrahydro-2H-pyran-2-yloxy)methanesulfonamide (Compound 39)

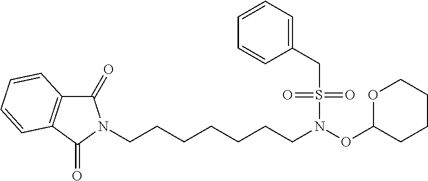

General procedure 9. Starting materials: compounds 38 and 5. ¹H-NMR (CDCl₃): δ 7.84 (m, 2H), 7.71 (m, 2H), 7.39 (m, 5H), 5.10 (m, 1H), 4.36 (q, 2H), 3.93 (m, 1H), 3.66 (t, 2H), 3.59 (m, 1H), 3.36 (m, 1H), 3.02 (m, 1H), 1.9-1.15 (m, 16H).

### Preparation 40

### N-(7-aminoheptyl)-1-phenyl-N-(tetrahydro-2H-pyran-2-yloxy)methanesulfonamide (Compound 40)

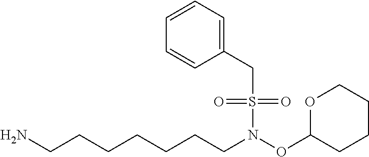

General procedure 4. Starting material: compound 39. ¹H-NMR (CD₃OD): δ 7.42 (m, 5H), 5.08 (m, 1H), 4.50 (q, 2H), 3.94 (m, 1H), 3.59 (m, 1H), 3.43 (m, 2H), 2.64 (t, 2H), 1.85-1.25 (m, 8H).

### Preparation 41

### N-(cyclohexylmethoxy)ethanesulfonamide (Compound 41)

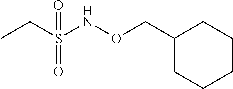

General procedure 8. Starting materials: O-cyclohexylmethylhydroxylamine (WO/2009/086835) and ethanesulfonyl chloride. ¹H-NMR (CDCl₃): δ 7.01 (s, 1H), 3.81 (d, 2H), 3.24 (q, 2H), 1.70 (m, 6H), 1.39 (m, 3H), 1.23 (m, 3H), 0.96 (m, 2H).

### Preparation 42

### N-(cyclohexylmethoxy)-N-(7-(1,3-dioxoisoindolin-2-yl)heptyl)ethanesulfonamide (Compound 42)

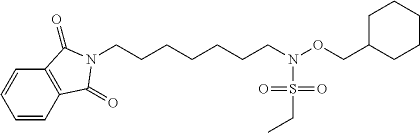

General procedure 9. Starting materials: compounds 41 and 5. ¹H-NMR (CDCl₃): δ 7.84 (m, 2H), 7.71 (m, 2H), 3.82 (d, 2H), 3.69 (t, 2H), 3.18 (t, 2H), 3.09 (q, 2H), 1.68 (m, 10H), 1.44 (t, 3H), 1.37 (m, 6H), 1.20 (m, 3H), 0.99 (m, 2H).

### Preparation 43

### N-(7-aminoheptyl)-N-(cyclohexylmethoxy)ethanesulfonamide (Compound 43)

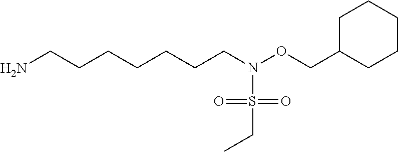

General procedure 4. Starting material: compound 42. ¹H-NMR (CDCl₃): δ 3.83 (d, 2H), 3.19 (t, 2H), 3.09 (q, 2H), 2.69 (t, 2H), 1.64 (m, 12H), 1.44 (t, 3H), 1.35 (m, 6H), 1.20 (m, 3H), 0.99 (m, 2H).

### Preparation 44

### N-(cyclohexyloxy)-4-fluorobenzensulfonamide (Compound 44)

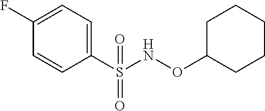

General procedure 8. Starting materials: O-cyclohexylhydroxylamine (see e.g. WO/2009/086835) and 4-fluorobenzenesulfonyl chloride. ¹H-NMR (CDCl₃): δ 7.96 (m, 2H), 7.25 (t, 2H), 6.68 (s, 1H), 3.99 (m, 1H), 1.98 (m, 2H), 1.71 (m, 2H), 1.29 (m, 6H).

### Preparation 45

### N-(cyclohexyloxy)-N-(7-(1,3-dioxoisoindolin-2-yl)heptyl)-4-fluorobenzenesulfonamide (Compound 45)

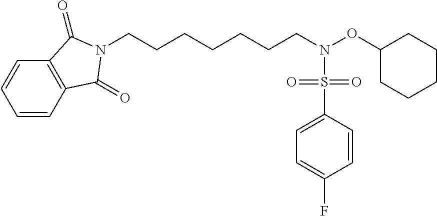

General procedure 9. Starting materials: compounds 44 and 5. ¹H-NMR (CDCl₃): δ 7.85 (m, 4H), 7.70 (m, 2H), 7.22 (t, 2H), 4.15 (m, 1H), 3.66 (t, 2H), 2.80 (bs, 2H), 2.07 (m, 2H), 1.75 (m, 2H), 1.65 (m, 2H), 1.58 (m, 4H), 1.4-1.05 (m, 10H).

### Preparation 46

### N-(7-aminoheptyl)-N-(cyclohexyloxy)-4-fluorobenzenesulfonamide (Compound 46)

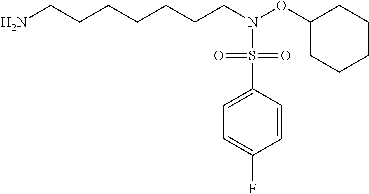

General procedure 4. Starting material: compound 45. ¹H-NMR (CDCl₃): δ 7.87 (m, 2H), 7.21 (t, 2H), 4.15 (m, 1H), 2.8 (bs, 2H), 2.66 (t, 2H), 2.07 (m, 2H), 1.77 (m, 4H), 1.55 (m, 4H), 1.41 (m, 2H), 1.24 (m, 10H).

### Preparation 47

### N-(3-morpholinopropyl)-2-nitrobenzenesulfonamide (Compound 47)

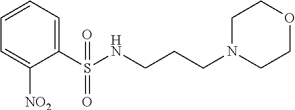

General procedure 8. Starting materials: 3-morpholinopropylamine and 2-nitrobenzene-1-sulfonyl chloride. ¹H-NMR (CDCl₃): δ 8.12 (m, 1H), 7.82 (m, 1H), 7.72 (m, 4H), 3.78 (t, 2H), 3.18 (t, 2H), 2.47 (m, 6H), 1.76 (m, 2H).

### Preparation 48

### N-(8-(1,3-dioxoisoindolin-2-yl)octyl)-N-(3-morpholinopropyl)-2-nitrobenzenesulfonamide (Compound 48)

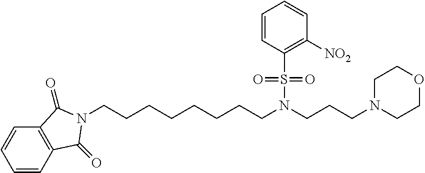

General procedure 9. Starting materials: compounds 47 and 3. ¹H-NMR (CDCl₃): δ 8.00 (m, 1H), 7.84 (m, 2H), 7.69 (m, 4H), 7.60 (m, 1H), 3.65 (m, 6H), 3.33 (t, 2H), 3.26 (t, 2H), 2.37 (t, 4H), 2.30 (t, 2H), 1.72 (m, 4H), 1.63 (m, 2H), 1.49 (m, 2H), 1.24 (m, 6H).

### Preparation 49

### 2-(8-(3-morpholinopropylamino)octyl)isoindoline-1,3-dione (Compound 49)

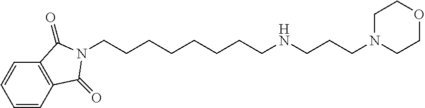

Compound 48 (176 mg, 3.0 mmol) was dissolved in CH₃CN, thiophenol (0.34 ml, 3.3 mmol) and Cs₂CO₃ (0.98 g, 3.0 mmol) were added and the mixture stirred at rt overnight, filtered, concentrated and purified by chromatography (chloroform:methanol:NH₃ (25% aq.) 98:2:0.2 to 95:5:1) to afford compound 49. ¹H-NMR (CDCl₃): δ 7.84 (m, 2H), 7.70 (m, 2H), 3.69 (m, 6H), 2.66 (t, 2H), 2.58 (t, 2H), 2.42 (m, 4H), 2.40 (t, 2H), 1.80 (bs, 1H), 1.68 (m, 4H), 1.47 (m, 2H), 1.30 (m, 8H).

### Preparation 50

### N-(8-(1,3-dioxoisoindolin-2-yl)octyl)-N-(3-morpholinopropyl)benzamide (Compound 50)

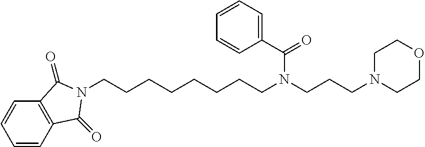

Benzoyl chloride (1.02 eq.) was added to a solution of compound 49 (1.0 eq.) and triethyamine (1.1 eq.) in DCM at 0° C. with stirring. The mixture was gradually allowed to reach rt, and after 2 h the mixture was concentrated and purified by chromatography (MeOH:CHCl₃:NH₃ (25% aq.) 98:2:0.2) to yield compound 50. ¹H-NMR (CDCl₃): δ ¹H-NMR (CDCl₃): δ 7.83 (m, 2H), 7.71 (m, 2H), 7.36 (m, 5H), 3.69 (bs, 4H), 3.51 (bs, 4H), 3.21 (bs, 2H), 2.47 (bs, 4H), 2.19 (bs, 2H), 1.95-1.0 (m, 14H).

### Preparation 51

### N-(8-aminooctyl)-N-(3-morpholinopropyl)benzamide (Compound 51)

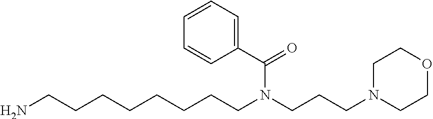

General procedure 4. Starting material: compound 50. Used without NMR-data.

### Preparation 52

### 3-cyclohexyl-1-(8-(1,3-dioxoisoindolin-2-yl)octyl)-1-(3-morpholinopropyl)urea (Compound 52)

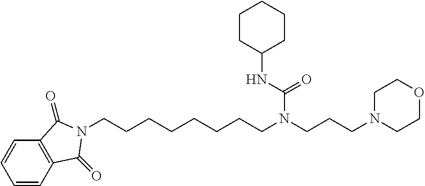

Cyclohexyl isocyanate (1.02 eq.) was added to a solution of compound 49 (1.0 eq.) and triethyamine (1.1 eq.) in DCM with stirring. The mixture was stirred at rt overnight, concentrated and purified by chromatography (MeOH:CHCl₃:NH₃ (25% aq.) 98:2:0.2) to yield compound 52. ¹H-NMR (CDCl₃): δ 7.83 (m, 2H), 7.70 (m, 2H), 5.16 (d, 1H), 3.73 (t, 4H), 3.66 (t, 2H), 3.59 (m, 1H), 3.20 (t, 2H), 3.13 (t, 2H), 2.43 (t, 4H), 2.33 (t, 2H), 1.94 (m, 2H), 1.67 (m, 8H), 1.51 (m, 2H), 1.31 (m, 9H), 1.08 (m, 3H).

### Preparation 53

### 1-(8-aminooctyl)-3-cyclohexyl-1-(3-morpholinopropyl)urea (Compound 53)

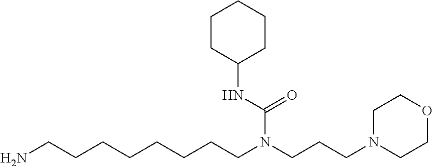

General procedure 4. Starting material: compound 52. ¹H-NMR (CDCl₃): δ 4.34 (d, 1H), 3.70 (m, 4H), 3.49 (m, 1H), 3.13 (t, 2H), 2.72 (t, 2H), 2.62 (t, 2H), 2.44 (m, 4H), 2.32 (t, 2H), 1.92 (m, 2H), 1.81 (m, 2H), 1.8-0.85 (m, 20H).

### Preparation 54

### O-Benzyl-N-ethylhydroxylamine (Compound 54)

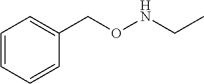

A solution of O-benzylhydroxylamine hydrochloride (2.0 g, 12.5 mmol), sodium acetate (2.0 g, 24.4 mmol) and acetaldehyde (1.44 ml, 25.5 mmol) in H₂O-MeOH (200 ml, 5:1 mixture) was stirred at room temperature for 10 min. The reaction mixture was extracted with EtOAc (2×200 ml), washed with 10% citric acid (400 ml), and dried (Na₂SO₄). The solvents were evaporated to give the crude acetaldehyde O-benzyloxime (2.3 g) as a 1:1 mixture of E and Z isomers which was used in the next step without further purification. ¹H-NMR (CDCl₃, HMDSO) δ: 1.84 (d, J=5.8 Hz, 0.5H); 1.87 (d, J=5.5 Hz, 0.5H); 5.04 (s, 1H); 5.11 (s, 1H); 6.79 (q, J=5.5 Hz, 0.5H); 7.23-7.39 (m, 5H); 7.48 (q, J=5.8 Hz, 0.5H). GC-MS (m/z): 149, 134, 119, 105, 91, 77.

Acetaldehyde O-benzyloxime from the previous step was dissolved in CH₂Cl₂ (60 ml) and solid NaCNBH₃ (2.66 g, 42 mmol) followed by 2N HCl solution in methanol (36 ml) were added. The reaction mixture was stirred overnight and evaporated. The residue was suspended in CH₂Cl₂ (25 ml) and 1N NaOH solution was added until the pH of the medium was 9. The organic layer was separated and the aqueous layer was washed with CH₂Cl₂ (2×50 ml). The organic extracts were combined, dried (Na₂SO₄), and evaporated. The residue was purified by FC with petroleum ether-EtOAc (gradient from 90:10 to 20:80) as eluent to give the title compound 54 as a colorless oil. ¹H-NMR (200 MHz, CDCl₃, HMDSO): δ 1.10 (t, J=7.1 Hz, 3H); 2.98 (q, J=7.1 Hz, 2H); 4.71 (s, 2H); 5.48 (b s, 1H); 7.23-7.40 (m, 5H).

### Preparation 55

### 2-(8-(benzyloxy(ethyl)amino)octyl)isoindoline-1,3-dione (Compound 55)

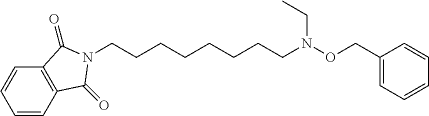

A mixture of compound 54 (0.85 g, 5.62 mmol), Na₂CO₃ (0.95 g, 8.99 mmol), and compound 3 (2.16 g, 6.39 mmol) in CH₃CN (40 ml) was stirred under reflux for 48 h, cooled, and poured onto ice-water (300 ml). The mixture was extracted with CH₂Cl₂ (3×100 ml), the combined extracts were dried (Na₂SO₄), and concentrated. The residue was purified by column chromatography (petroleum ether-EtOAc (5:1)) to afford a 2:1 mixture of the title compound 55 with the starting amine 54 as a colourless oil which was used in the next step without further purification. ¹H-NMR (200 MHz, CDCl₃, HMDSO): δ 1.15 (t, J=7.1 Hz, 3H); 1.22-1.41 (m, 8H); 1.47-1.76 (m, 4H); 2.74 (q, J=7.1 Hz, 2H); 3.67 (t, J=7.2 Hz, 2H); 4.69 (s, 2H); 7.23-7.39 (m, 5H), 7.64-7.75 (m, 2H); 7.78-7.88 (m, 2H).

### Preparation 56

### 8-(benzyloxy(ethyl)amino)octane-1-amine (Compound 56)

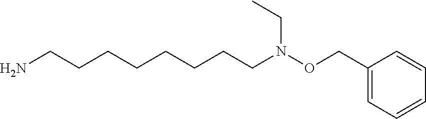

Compound 55 (1.210 g, containing ca 0.810 g (1.98 mmol) of 54) was dissolved in EtOH (30 ml), hydrazine hydrate (0.29 ml, 5.92 mmol) was added, and the obtained solution was refluxed for 3 h. The reaction mixture was cooled, precipitated solid was filtered off, and the filtrate was concentrated. The residue was purified by chromatography (CH₂Cl₂-MeOH—NH₄OH (75:8:1)) to afford compound 56 as a colorless oil. ¹H-NMR (200 MHz, CDCl₃, HMDSO): δ 1.16 (t, J=7.1 Hz, 3H); 1.23-1.50 (m, 12H); 1.50-1.68 (m, 2H); 2.67 (t, J=7.0 Hz, 2H); 2.67 (t, J=7.0 Hz, 2H); 2.75 (q, J=7.1 Hz, 2H); 4.70 (s, 2H); 7.23-7.39 (m, 5H).

### Preparation 57

### N-Benzyl-O-ethylhydroxylamine (Compound 57)

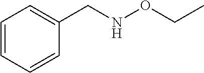

Prepared as described for compound 54 using O-ethylhydroxylamine hydrochloride and benzaldehyde.

¹H-NMR (400 MHz, CDCl₃, HMDSO): δ 1.13 (t, J=7.0 Hz, 3H); 3.69 (q, J=7.0 Hz, 2H); 4.04 (s, 2H); 5.58 (b s, 1H); 7.24-7.38 (m, 5H).

### Preparation 58

### 2-(8-(benzyl(ethoxy)amino)octyl)isoindoline-1,3-dione (Compound 58)

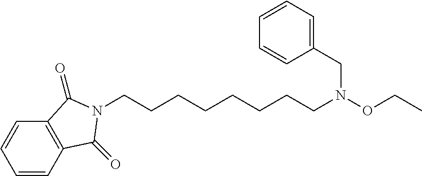

Prepared as described for compound 55 using compound 57 and compound 3. ¹H-NMR (400 MHz, CDCl₃, HMDSO): δ 0.99 (t, J=7.0 Hz, 3H); 1.22-1.37 (m, 8H); 1.55 (qui, J=7.2 Hz, 2H); 1.65 (qui, J=7.2 Hz, 2H); 2.63 (t, J=7.3 Hz, 2H); 3.50 (q, J=7.0 Hz, 2H); 3.66 (t, J=7.4 Hz, 2H); 3.78 (s, 2H); 7.21-7.37 (m, 5H); 7.66-7.72 (m, 2H); 7.80-7.86 (m, 2H).

### Preparation 59

### 8-(benzyl(ethoxy)amino)octane-1-amine (Compound 59)

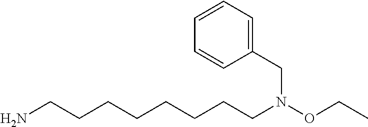

Prepared as described for compound 56 using compound 58. ¹H-NMR (400 MHz, CDCl₃, HMDSO): δ 1.00 (t, J=7.0 Hz, 3H); 1.28 (m, 8H); 1.36 (b s, 2H); 1.42 (qui, J=7.0 Hz, 2H); 1.56 (qui, J=7.2 Hz, 2H); 2.64 (t, J=7.4 Hz, 2H); 2.67 (t, J=7.0 Hz, 2H); 3.51 (q, J=7.0 Hz, 2H); 3.79 (s, 2H); 7.25 (m, 1H); 7.30 (t, J=7.3 Hz, 2H); 7.34 (d, J=7.2 Hz, 2H).

### Preparation 60

### N-Ethyl-O-(2-morpholinoethyl)hydroxylamine (Compound 60)

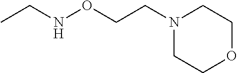

Prepared as described for compound 54 using O-(2-morpholinoethyl)hydroxylamine (see, e.g. WO/2009/086835) and acetaldehyde.

¹H-NMR (200 MHz, DMSO-d₆): δ 0.96 (t, J=7.1 Hz, 3H); 2.38 (m, 4H); 2.45 (t, J=5.9 Hz, 2H); 2.77 (q, J=7.1 Hz, 2H); 3.54 (m, 4H); 3.66 (t, J=5.9 Hz, 2H)

### Preparation 61

### 2-(8-(ethyl(2-morpholinoethoxy)amino)octyl)isoindoline-1,3-dione (Compound 61)

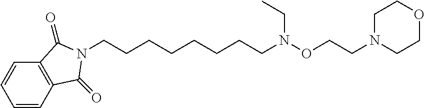

Prepared as described for compound 55 using compound 60 and compound 3. ¹H-NMR (400 MHz, CDCl₃, HMDSO): δ 1.11 (t, J=7.1 Hz, 3H); 1.22-1.37 (m, 8H); 1.53 (qui, J=7.2 Hz, 2H); 1.66 (qui, J=7.1 Hz, 2H); 2.48 (m, 4H); 2.54 (t, J=5.9 Hz, 2H); 2.62 (t, J=7.5 Hz, 2H); 2.70 (q, J=7.1 Hz, 2H); 3.67 (t, J=7.4 Hz, 2H); 3.71 (m, 4H); 3.82 (t, J=5.9 Hz, 2H); 7.67-7.73 (m, 2H); 7.80-7.86 (m, 2H).

### Preparation 62

### 8-(ethyl(2-morpholinoethoxy)amino)octane-1-amine (Compound 62)

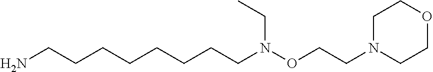

Prepared as described for compound 56 using compound 61. ¹H-NMR (400 MHz, CDCl₃, HMDSO): δ 1.11 (t, J=7.1 Hz, 3H); 1.23-1.36 (m, 10H); 1.43 (qui, J=7.0 Hz, 2H); 1.54 (qui, J=7.1 Hz, 2H); 2.48 (m, 4H); 2.54 (t, J=5.9 Hz, 2H); 2.63 (t, J=7.5 Hz, 2H); 2.67 (t, J=7.0 Hz, 2H); 2.71 (q, J=7.1 Hz, 2H); 3.71 (m, 4H); 3.83 (t, J=5.9 Hz, 2H).

### Preparation 63

### N¹-(3-morpholinopropyl)octane-1,8-diamine (Compound 63)

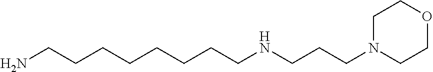

General procedure 4. Starting material: compound 49. ¹H-NMR (CD₃OD): δ 3.70 (m, 4H), 3.00 (t, 2H), 2.89 (m, 4H), 2.48 (m, 6H), 1.86 (m, 2H), 1.63 (m, 4H), 1.37 (m, 8H).

### Preparation 64

### N-(8-(1,3-dioxoisoindolin-2-yl)octyl)-P,P-dimethyl-N-(3-morpholinopropyl)phosphinic amide (Compound 64)

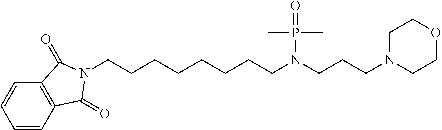

Compound 49 (229 mg, 0.6 mmol) and NEt₃ (0.09 ml, 0.63 mmol) were dissolved in DCM and cooled on an icebath, and dimethyl phosphinic chloride (64 mg, 0.63 mmol) was added with stirring. The reaction mixture was gradually allowed to reach rt and stirred overnight, concentrated and purified by chromatography (chloroform:methanol:NH₃ (25% aq.) 98:2:0.2 to 95:5:1) to afford compound 64. ¹H-NMR (CDCl₃): δ 7.83 (m, 2H), 7.71 (m, 2H), 3.69 (m, 4H), 3.66 (t, 2H), 3.00 (m, 2H), 2.91 (m, 2H), 2.42 (m, 4H), 2.32 (t, 2H), 1.67 (m, 4H), 1.49 (m, 2H), 1.47 (s, 3H), 1.42 (s, 3H), 1.29 (m, 8H).

### Preparation 65

### N-(8-aminooctyl)-P,P-dimethyl-N-(3-morpholinopropyl)phosphinic amide (Compound 65)

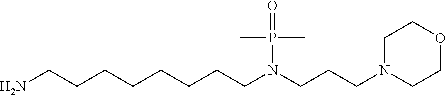

General procedure 4. Starting material: compound 64. ¹H-NMR (CD₃OD): δ 3.70 (m, 4H), 3.01 (m, 4H), 2.64 (t, 2H), 2.47 (m, 4H), 2.38 (t, 2H), 1.75 (m, 2H), 1.65-1.40 (m, 10H), 1.36 (m, 8H).

### Preparation 66

### N-(8-aminooctyl)-N-(3-morpholinopropyl)hydrazinecarboxamide (Compound 66)

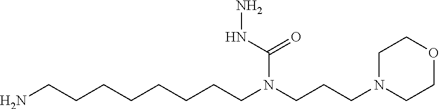

General procedure 4. Starting material: compound 52 (compound 66 was obtained as a byproduct). ¹H-NMR (CDCl₃): δ 6.48 (bs, 2H), 6.04 (t, 1H), 3.70 (bs, 2H), 3.67 (t, 4H), 3.17 (m, 2H), 2.62 (t, 2H), 2.55 (t, 2H), 2.40 (m, 4H), 2.36 (t, 2H), 1.65 (m, 2H), 1.55 (m, 4H), 1.27 (m, 8H).

### Preparation 67

### N-(2-fluoroethyl)cyclohexansulfonamide (Compound 67)

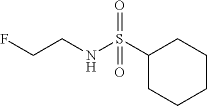

General procedure 8. Starting materials: 2-fluoroethanamine and cyclohexanesulfonyl chloride. ¹H-NMR (CDCl₃): δ 4.53 (dt, 2H), 3.45 (dt, 2H), 2.89 (m, 1H), 2.36 (bs, 1H), 2.3-1.15 (m, 10H).

### Preparation 68

### N-(7-(1,3-dioxoisoindolin-2-yl)heptyl)-N-(2-fluoroethyl)cyclohexanesulfonamide (Compound 68)

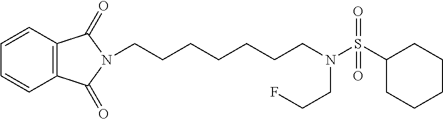

General procedure 9. Starting materials: compounds 67 and 5. ¹H-NMR (CDCl₃): δ 7.83 (m, 2H), 7.71 (m, 2H), 4.53 (dt, 2H), 3.66 (t, 2H), 3.53 (dt, 2H), 3.25 (t, 2H), 2.91 (m, 1H), 2.08 (m, 2H), 1.87 (m, 2H), 1.75-1.1 (m, 16H).

### Preparation 69

### N-(7-aminoheptyl)-N-(2-fluoroethyl)cyclohexanesulfonamide (Compound 69)

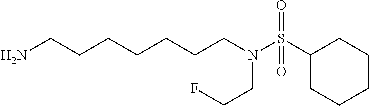

General procedure 4. Starting material: compound 68. ¹H-NMR (CD₃OD): δ 4.56 (dt, 2H), 3.57 (dt, 2H), 3.30 (t, 2H), 3.10 (m, 1H), 2.65 (t, 2H), 2.10 (m, 2H), 1.89 (m, 2H), 1.8-1.1 (m, 16H).

### Preparation 70

### N-(7-(1,3-dioxoisoindolin-2-yl)octyl)-N-(2-fluoroethyl)cyclohexanesulfonamide (Compound 70)

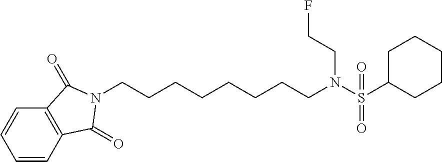

General procedure 9. Starting materials: compounds 67 and 3. ¹H-NMR (CDCl₃): δ 7.83 (m, 2H), 7.71 (m, 2H), 4.53 (dt, 2H), 3.65 (t, 2H), 3.53 (dt, 2H), 3.24 (t, 2H), 2.89 (m, 1H), 2.08 (m, 2H), 1.87 (m, 2H), 1.75-1.4 (m, 8H), 1.4-1.1 (m, 10H).

### Preparation 71

### N-(7-aminooctyl)-N-(2-fluoroethyl)cyclohexanesulfonamide (Compound 71)

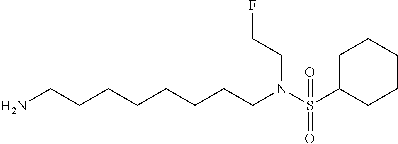

General procedure 4. Starting material: compound 70. ¹H-NMR (CD₃OD): δ 4.55 (dt, 2H), 3.57 (dt, 2H), 3.30 (t, 2H), 3.10 (m, 1H), 2.68 (t, 2H), 2.10 (m, 2H), 1.89 (m, 2H), 1.8-1.1 (m, 18H).

### Preparation 72

### N-(cyclohexylmethoxy)-2-nitrobenzenesulfonamide (Compound 72)

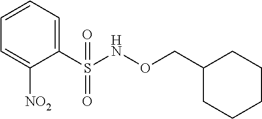

General procedure 8. Starting materials: O-(cyclohexylmethyl)hydroxylamine (see e.g. (WO/2009/086835)) and 2-nitrobenzene-1-sulfonyl chloride. ¹H-NMR (CDCl₃): δ 8.21 (m, 1H), 8.06 (bs, 1H), 7.90 (m, 1H), 7.81 (m, 2H), 3.90 (d, 2H), 1.67 (m, 6H), 1.20 (m, 3H), 0.94 (m, 2H).

### Preparation 73

### N-(cyclohexylmethoxy)-N-(8-(1,3-dioxoisoindolin-2-yl)octyl)-2-nitrobenzenesulfonamide (Compound 73)

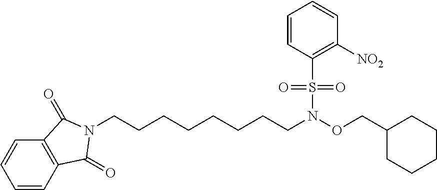

General procedure 9. Starting materials: compounds 72 and 3. ¹H-NMR (CDCl₃): δ 8.02 (m, 1H), 7.82 (m, 2H), 7.77 (m, 1H), 7.72 (m, 1H), 7.69 (m, 2H), 7.55 (m, 1H), 3.87 (d, 2H), 3.65 (t, 2H), 3.05 (t, 2H), 1.8-1.5 (m, 10H), 1.4-1.05 (m, 11H), 0.98 (m, 2H).

### Preparation 74

### 2-(8-(cyclohexylmethoxyamino)octyl)isoindolin-1,3-dione (Compound 74)

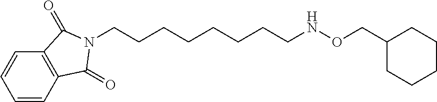

Compound 73 (236 mg, 0.41 mmol) was dissolved in CH₃CN, thiophenol (0.06 ml, 0.57 mmol) and Cs₂CO₃ (0.40 g, 1.23 mmol) were added and the mixture stirred at rt overnight, filtered, concentrated and purified by chromatography (1% methanol in DCM) to afford compound 74. ¹H-NMR (CDCl₃): 7.84 (m, 2H), 7.70 (m, 2H), 3.67 (t, 2H), 3.46 (d, 2H), 2.88 (t, 2H), 1.8-1.0 (m, 22H), 0.91 (m, 2H).

### Preparation 75

### 8-(cyclohexylmethoxyamino)octan-1-amine (Compound 75)

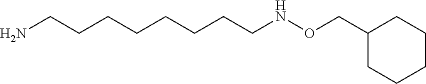

General procedure 4. Starting material: compound 74. ¹H-NMR (CDCl₃): 3.44 (d, 2H), 2.86 (t, 2H), 2.64 (t, 2H), 1.75-1.0 (m, 24H), 0.89 (m, 2H).

### Preparation 76

### N-(cyclohexylmethoxy)-N-(8-(1,3-dioxoisoindolin-2-yl)heptyl)-2-nitrobenzenesulfonamide (Compound 76)

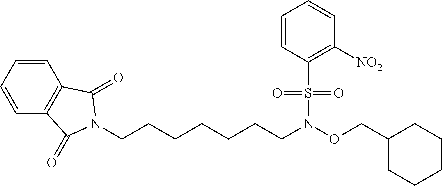

General procedure 9. Starting materials: compounds 72 and 5. ¹H-NMR (CDCl₃): δ 8.04 (dd, 1H), 7.83 (m, 2H), 7.77 (m, 1H), 7.70 (m, 3H), 7.56 (dd, 1H), 3.88 (d, 2H), 3.67 (t, 2H), 3.06 (t, 2H), 1.8-1.5 (m, 10H), 1.4-1.05 (m, 9H), 0.99 (m, 2H).

### Preparation 77

### 2-(8-(cyclohexylmethoxyamino)heptyl)isoindolin-1,3-dione (Compound 77)

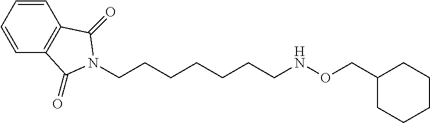

Compound 76 (440 mg, 0.79 mmol) was dissolved in CH₃CN, thiophenol (0.09 ml, 0.87 mmol) and Cs₂CO₃ (0.77 g, 2.37 mmol) were added and the mixture stirred at rt overnight, filtered, concentrated and purified by chromatography (1% methanol in DCM) to afford compound 77. ¹H-NMR (CDCl₃): 7.84 (m, 2H), 7.70 (m, 2H), 3.67 (t, 2H), 3.46 (d, 2H), 2.88 (t, 2H), 1.8-1.05 (m, 20H), 0.91 (m, 2H).

### Preparation 78

### 8-(cyclohexylmethoxyamino)heptan-1-amine (Compound 78)

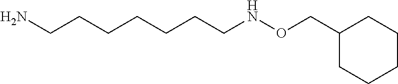

General procedure 4. Starting material: compound 77. ¹H-NMR (CDCl₃): 3.47 (d, 2H), 2.89 (t, 2H), 2.68 (t, 2H), 1.85-1.0 (m, 22H), 0.92 (m, 2H).

### Preparation 79

### N-(6-hydroxyhexyl)phthalimide (Compound 79)

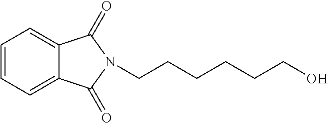

General procedure 1. Starting material: 6-bromohexan-1-ol. Used without NMR-data.

### Preparation 80

### N-(6-bromohexyl)phthalimide (Compound 80)

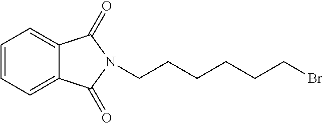

General procedure 2. Starting material: compound 79. ¹H-NMR (CDCl₃): δ 7.88-7.78 (m, 2H), 7.76-7.65 (m, 2H), 3.68 (t, 2H), 3.39 (t, 2H), 1.85 (m, 2H), 1.69 (m, 2H), 1.56-1.27 (m, 4H).

### Preparation 81

### N-(cyclohexylmethoxy)-N-(6-(1,3-dioxoisoindolin-2-yl)hexyl)-2-nitrobenzenesulfonamide (Compound 81)

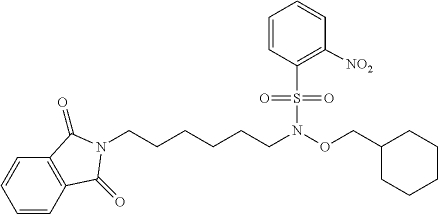

General procedure 9. Starting materials: compounds 72 and 80. ¹H-NMR (CDCl₃): δ 8.03 (dd, 1H), 7.84 (m, 2H), 7.75 (m, 1H), 7.71 (m, 3H), 7.55 (dd, 1H), 3.88 (d, 2H), 3.67 (t, 2H), 3.07 (t, 2H), 1.8-1.5 (m, 10H), 1.4-1.05 (m, 7H), 0.96 (m, 2H).

### Preparation 82

### 2-(6-(cyclohexylmethoxyamino)hexyl)isoindolin-1,3-dione (Compound 82)

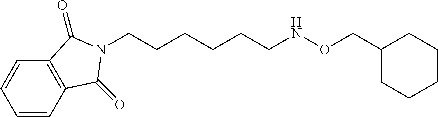

Compound 81 (463 mg, 0.85 mmol) was dissolved in CH₃CN, thiophenol (0.11 ml, 1.02 mmol) and Cs₂CO₃ (0.83 g, 2.55 mmol) were added and the mixture stirred at rt overnight, filtered, concentrated and purified by chromatography (1% methanol in DCM) to afford compound 77. ¹H-NMR (CDCl₃): 7.84 (m, 2H), 7.70 (m, 2H), 3.68 (t, 2H), 3.46 (d, 2H), 2.88 (t, 2H), 1.8-1.05 (m, 18H), 0.92 (m, 2H).

### Preparation 83

### 6-(cyclohexylmethoxyamino)hexan-1-amine (Compound 83)

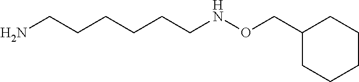

General procedure 4. Starting material: compound 82. ¹H-NMR (CDCl₃): 3.47 (d, 2H), 2.89 (t, 2H), 2.68 (t, 2H), 1.85-1.0 (m, 20H), 0.91 (m, 2H).

### Preparation 84

### N-(6-(1,3-dioxoisoindolin-2-yl)hexyl)-N-(2-fluoroethyl)cyclohexanesulfonamide (Compound 84)

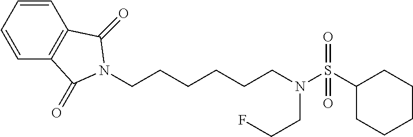

General procedure 9. Starting materials: compounds 67 and 80. ¹H-NMR (CDCl₃): δ 7.81 (m, 2H), 7.69 (m, 2H), 4.50 (dt, 2H), 3.55 (t, 2H), 3.55 (dt, 2H), 3.23 (t, 2H), 2.87 (m, 1H), 2.15 (m, 2H), 1.86 (m, 2H), 1.75-1.1 (m, 14H).

### Preparation 85

### N-(6-aminohexyl)-N-(2-fluoroethyl)cyclohexanesulfonamide (Compound 85)

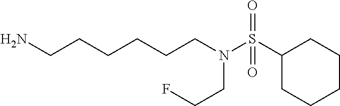

General procedure 4. Starting material: compound 84. ¹H-NMR (CDCl₃): δ 4.51 (dt, 2H), 3.51 (dt, 2H), 3.24 (t, 2H), 2.86 (m, 1H), 2.65 (t, 2H), 2.06 (m, 2H), 1.85 (m, 2H), 1.75-1.1 (m, 14H).

### Preparation 86

### 2-(7-morpholinoheptyl)isoindoline-1,3-dione (Compound 86)

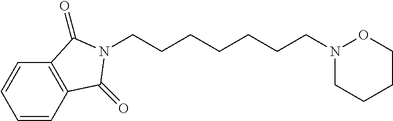

Compound 80 (648 mg, 2 mmol) and tetrahydro-1,2-oxazin-2-ium chloride (J. Chem. Soc., Pekin Trans 2 (2000), 1435-144) (272 mg, 2.2 mmol) were dissolved in DMF (6 ml), Cs₂CO₃ (1.955 g, 6 mmol) was added and the mixture stirred at 70° C. overnight, filtered, concentrated and purified by chromatography (petroleum ether:EtOAc 5:1 to 2:1) to afford compound 86. ¹H-NMR (CDCl₃): δ 7.84 (m, 2H), 7.71 (m, 2H), 3.90 (t, 2H), 3.67 (t, 2H), 2.71 (bs, 2H), 2.57 (t, 2H), 1.77 (m, 2H), 1.66 (m, 2H), 1.54 (m, 4H), 1.34 (m, 6H).

### Preparation 87

### 7-morpholinoheptan-1-amine (Compound 87)

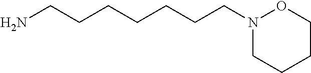

General procedure 4. Starting material: compound 86. ¹H-NMR (CDCl₃): δ 3.90 (t, 2H), 2.71 (bs, 2H), 2.67 (t, 2H), 2.58 (t, 2H), 1.52 (m, 2H), 1.66 (m, 6H), 1.52 (m, 2H), 1.31 (m, 6H).

### Preparation 88

### N-(5-hydroxypentyl)phthalimide (Compound 88)

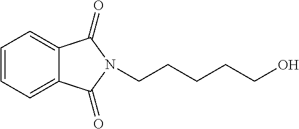

Phtalic anhydride (7.6 g, 51.3 mmol) and 5-amino-pentan-1-ol (5.0 ml, 53.9 mmol) were heated to 140° C. overnight, cooled to rt, extracted with EtOAc/NaHCO₃ (aq., sat.). The organic phase was subsequently washed with water, 10% citric acid, brine, dried (MgSO₄) and concentrated to yield compound 88. ¹H-NMR (CDCl₃): δ 7.83 (m, 2H), 7.70 (m, 2H), 3.69 (t, 2H), 3.63 (t, 2H), 1.71 (m, 2H), 1.60 (m, 3H), 1.41 (m, 2H).

### Preparation 89

### N-(5-bromopentyl)phthalimide (Compound 89)

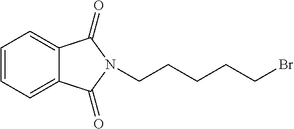

General procedure 2. Starting material: compound 88. ¹H-NMR (CDCl₃): δ 7.84 (m, 2H), 7.71 (m, 2H), 3.69 (t, 2H), 3.39 (t, 2H), 1.91 (m, 2H), 1.71 (m, 2H), 1.49 (m, 2H).

### Preparation 90

### N-(cyclohexylmethoxy)-N-(5-(1,3-dioxoisoindolin-2-yl)pentyl)-2-nitrobenzenesulfonamide (Compound 90)

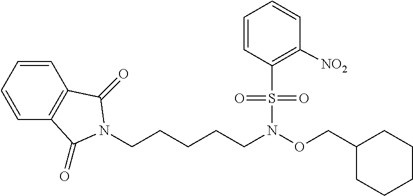

General procedure 9. Starting materials: compounds 72 and 89. ¹H-NMR (CDCl₃): δ 8.04 (dd, 1H), 7.84 (m, 2H), 7.73 (m, 4H), 7.56 (dd, 1H), 3.88 (d, 2H), 3.68 (t, 2H), 3.09 (t, 2H), 1.70 (m, 10H), 1.44 (m, 2H), 1.23 (m, 3H), 0.98 (m, 2H).

### Preparation 91

### 2-(5-(cyclohexylmethoxyamino)pentyl)isoindolin-1,3-dione (Compound 91)

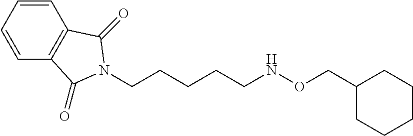

Compound 90 (537 mg, 1.01 mmol) was dissolved in CH₃CN, thiophenol (0.28 ml, 2.02 mmol) and Cs₂CO₃ (0.99 g, 3.03 mmol) were added and the mixture stirred at rt overnight, filtered, concentrated and purified by chromatography (1% methanol in DCM) to afford compound 91. ¹H-NMR (CDCl₃): 7.84 (m, 2H), 7.70 (m, 2H), 5.39 (bs, 1H), 3.69 (t, 2H), 3.45 (d, 2H), 2.89 (t, 2H), 1.8-1.05 (m, 15H), 0.91 (m, 2H).

### Preparation 92

### 5-(cyclohexylmethoxyamino)pentan-1-amine (Compound 92)

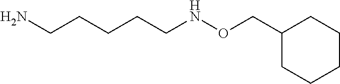

General procedure 4. Starting material: compound 91. ¹H-NMR (CD₃OD): 3.48 (d, 2H), 2.87 (t, 2H), 2.65 (t, 2H), 1.85-1.1 (m, 15H), 0.98 (m, 2H).

### Preparation 93

### N-(benzyloxy)-N-(6-(1,3-dioxoisoindolin-2-yl)hexyl)methanesulfonamide (Compound 93)

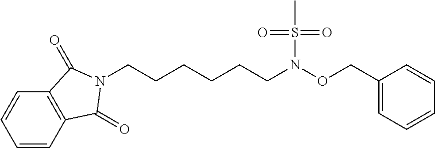

General procedure 9. Starting materials: compounds 22 and 80. ¹H-NMR (CDCl₃): δ 7.85 (dd, 2H), 7.72 (dd, 2H), 7.36 (m, 5H), 5.02 (s, 2H), 3.68 (t, 2H), 3.14 (m, 2H), 2.88 (s, 3H), 1.67 (m, 2H), 1.59 (m, 2H), 1.36 (m, 4H).

### Preparation 94

### N-(6-aminohexyl)-N-(benzyloxy)methanesulfonamide (Compound 94)

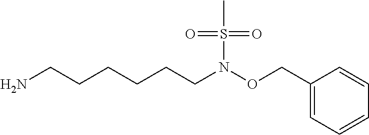

General procedure 4. Starting material: compound 93. ¹H-NMR (CD₃OD): δ 7.40 (m, 5H), 5.02 (s, 2H), 3.19 (t, 2H), 2.96 (s, 3H), 2.65 (t, 2H), 1.60 (m, 2H), 1.49 (m, 2H), 1.37 (m, 4H).

### Preparation 95

### N-(benzyloxy)-N-(7-(1,3-dioxoisoindolin-2-yl)heptyl)methanesulfonamide (Compound 95)

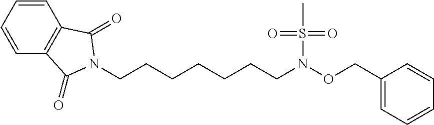

General procedure 9. Starting materials: compounds 22 and 5. ¹H-NMR (CDCl₃): δ 7.84 (dd, 2H), 7.71 (dd, 2H), 7.37 (m, 5H), 5.02 (s, 2H), 3.68 (t, 2H), 3.14 (m, 2H), 2.88 (s, 3H), 1.63 (m, 4H), 1.33 (m, 6H).

### Preparation 96

### N-(7-aminoheptyl)-N-(benzyloxy)methanesulfonamide (Compound 96)

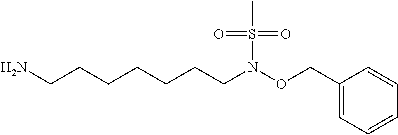

General procedure 4. Starting material: compound 95. ¹H-NMR (CD₃OD): δ 7.40 (m, 5H), 5.02 (s, 2H), 3.18 (t, 2H), 2.96 (s, 3H), 2.66 (t, 2H), 1.59 (m, 2H), 1.50 (m, 2H), 1.34 (m, 6H).

### Preparation 97

### N-(4-fluorobenzyloxy)methanesulfonamide (Compound 97)

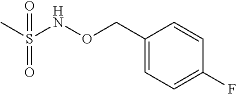

General procedure 8. Starting materials: O-(4-fluorobenzyl)hydroxylamine and methanesulfonyl chloride. ¹H-NMR (CDCl₃): δ 7.38 (m, 2H), 7.07 (m, 2H), 6.86 (bs, 1H), 4.96 (s, 2H), 3.04 (s, 3H).

### Preparation 98

### N-(8-(1,3-dioxoisoindolin-2-yl)octyl)-N-(4-fluorobenzyloxy)-methanesulfonamide (Compound 98)

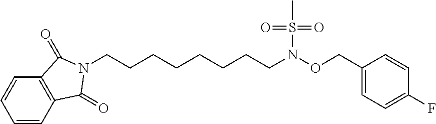

General procedure 9. Starting materials: compounds 97 and 3. ¹H-NMR (CDCl₃): δ 7.85 (dd, 2H), 7.71 (dd, 2H), 7.38 (m, 2H), 7.05 (m, 2H), 4.99 (s, 2H), 3.68 (t, 2H), 3.13 (t, 2H), 2.88 (s, 3H), 1.62 (m, 4H), 1.32 (m, 8H).

### Preparation 99

### N-(8-aminooctyl)-N-(4-fluorobenzyloxy)methanesulfonamide (Compound 99)

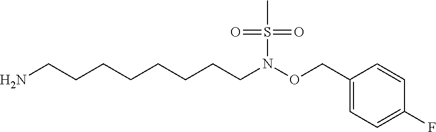

General procedure 4. Starting material: compound 98. ¹H-NMR (CD₃OD): δ 7.45 (m, 2H), 7.12 (m, 2H), 5.00 (s, 2H), 3.17 (t, 2H), 2.96 (s, 3H), 2.66 (t, 2H), 1.53 (m, 4H), 1.33 (m, 8H).

### EXAMPLES

### Example 1

### 2-cyano-1-(7-(cyclohexyl(3-morpholinopropyl)amino)octyl)-3-(pyridin-4-yl)guanidine (Compound 1001)

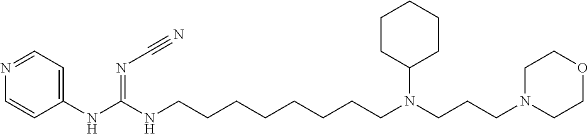

General procedure 5. Starting materials: S-Methyl N-cyano-N′-4-pyridylisothiourea and compound 9. ¹H-NMR (CD₃OD) δ: 3.39 (d, 2H), 7.94 (s, 1H, NH), 7.35 (bs, 2H), 3.71 (t, 4H), 3.40 (t, 2H), 2.60-2.47 (m, 9H), 2.39 (dd, 2H), 1.85-1.82 (m, 4H), 1.70-1.63 (m, 4H), 1.51-1.17 (m, 16H).

### Example 2

### 2-cyano-1-(7-(cyclohexyl(3-morpholinopropyl)amino)heptyl)-3-(pyridin-4-yl)guanidine (Compound 1002)

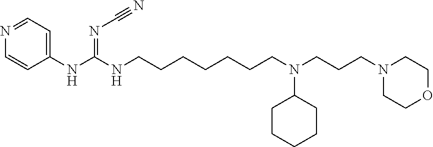

General procedure 5. Starting materials: S-Methyl N-cyano-N′-4-pyridylisothiourea and compound 10. ¹H-NMR (CDCl₃): δ 8.44 (d, 2H), 7.30 (d, 2H), 6.25 (bs, 1H, NH), 3.69 (t, 4H), 3.47 (bs, 2H), 2.69-2.55 (m, 4H), 2.43-2.33 (m, 7H), 1.87-1.09 (m, 22H).

### Example 3

### 2-cyano-1-(6-(cyclohexyl(3-morpholinopropyl)amino)hexyl)-3-(pyridin-4-yl)guanidine (Compound 1003)

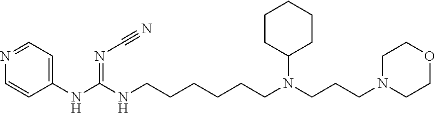

General procedure 5. Starting materials: S-Methyl N-cyano-N′-4-pyridylisothiourea and compound 11. ¹H-NMR (CDCl₃): δ 8.48 (d, 2H), 7.32 (d, 2H), 3.69 (t, 4H), 3.54-3.47 (m, 2H), 2.73-2.61 (m, 4H), 2.44-2.34 (m, 7H), 1.90-1.03 (m, 20H).

### Example 4

### 1-(8-(cyclohexyl(3-morpholinopropyl)amino)octyl)-3-(pyridin-3-ylmethyl)urea (Compound 1004)

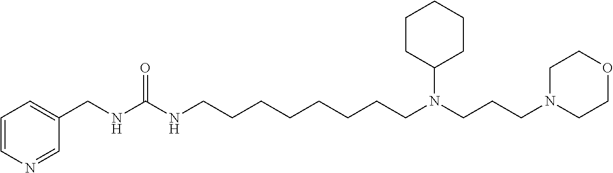

General procedure 6. Starting material: 3-picolylamine and compound 9. ¹H-NMR (CDCl₃): δ 8.52 (bs, 1H), 8.48 (d, 1H), 7.66 (d, 1H), 7.26-7.21 (m, 1H), 5.25 (bs, 1H, NH), 4.90 (bs, 1H, NH), 4.39 (d, 2H), 3.70 (t, 4H), 3.19 (q, 2H), 2.60-2.42 (m, 9H), 2.34 (t, 2H), 1.85-1.20 (m, 24H).

### Example 5

### 1-(7-(cyclohexyl(3-morpholinopropyl)amino)heptyl)-3-(pyridin-3-ylmethyl)urea (Compound 1005)

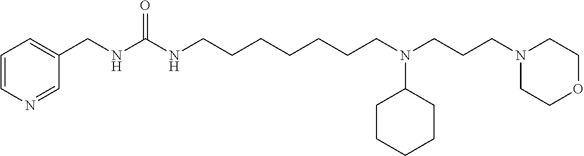

General procedure 6. Starting materials: 3-picolylamine and compound 10. ¹H-NMR (CD₃OD): δ 8.50 (bs, 1H), 8.43 (d, 1H), 7.80 (d, 1H), 7.44-7.40 (m, 1H), 4.37 (s, 2H), 3.71 (t, 4H), 3.14 (t, 2H), 2.62-2.37 (m, 11H), 1.85-1.16 (m, 22H).

### Example 6

### 3-(8-(cyclohexyl(3-morpholinopropyl)amino)-4-(pyridin-4-ylamino)cyclobut-3-ene-1,2-dione (Compound 1006)

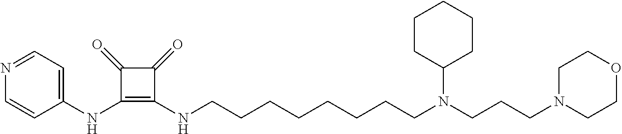

General procedure 7. Starting materials: 3-ethoxy-4-(pyridin-4-ylamino)cyclobut-3-ene-2,3-dione and compound 9. ¹H-NMR (CD₃OD) δ: 8.36 (d, 2H), 7.53 (d, 2H), 3.75-3.69 (m, 6H), 2.63-2.48 (m, 9H), 2.39 (t, 2H), 0.84-1.24 (m, 24H).

### Example 7

### 3-(7-cyclohexyl(3-morpholinopropyl)amino)heptylamino)-4-(pyridin-4-ylamino)cyclobut-3-ene-1,2-dione (Compound 1007)

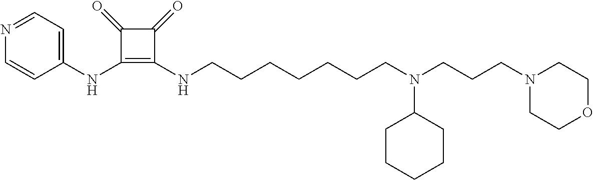

General procedure 7. Starting materials: 3-ethoxy-4-(pyridin-4-ylamino)cyclobut-3-ene-2,3-dione compound 10. ¹H-NMR (CD₃OD) δ: 8.38 (d, 1H), 7.57 (d, 1H), 3.77-3.70 (m, 6H), 3.10-2.90 (m, 5H), 2.53-2.46 (m, 6H), 1.98-1.18 (m, 22H).

### Example 8

### N-(8-(2-cyano-3-(pyridin-4-yl)guanidino)octyl)-N-(3-morpholinopropyl)cyclopentanesulfonamide (Compound 1008)

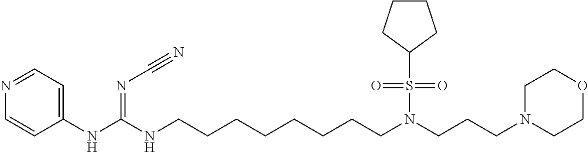

General procedure 5. Starting materials: S-Methyl N-cyano-N′-4-pyridylisothiourea and compound 20. ¹H-NMR (CDCl₃): δ 8.41 (d, 2H), 7.23 (d, 2H), 6.05 (t, 1H, NH), 3.67 (t, 4H), 3.47-3.32 (m, 3H), 3.25-3.14 (m, 4H), 2.39 (t, 4H), 2.31 (t, 2H), 1.95-1.89 (m, 4H), 1.78-1.71 (m, 4H), 1.60-1.51 (m, 6H), 1.33-1.28 (m, 8H).

### Example 9

### N-(8-(2-cyano-3-(pyridin-4-yl)guanidino)octyl)-N-(3-morpholinopropyl)cyclohexanesulfonamide (Compound 1009)

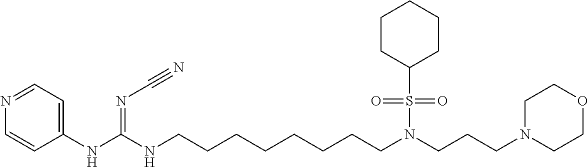

General procedure 5. Starting materials: S-Methyl N-cyano-N′-4-pyridylisothiourea and compound 21. ¹H-NMR (CDCl₃): δ 8.50 (d, 2H), 7.25 (d, 2H), 5.72 (bs, 1H, NH), 3.69 (t, 4H), 3.37 (q, 2H), 3.27-3.17 (m, 4H), 2.86 (tt, 1H), 2.42 (t, 4H), 2.33 (t, 2H), 2.04 (d, 2H), 1.89-1.18 (m, 22H).

### Example 10

### N-(7-(2-cyano-3-(pyridin-4-yl)guanidino)heptyl)-N-(3-morpholino propyl)cyclohexanesulfonamide (Compound 1010)

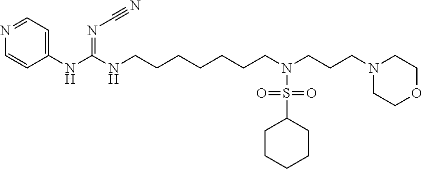

General procedure 5. Starting materials: S-Methyl N-cyano-N′-4-pyridylisothiourea and compound 19. ¹H-NMR (CDCl₃): δ 8.51 (d, 2H), 7.93 (bs, 1H, NH), 7.26 (d, 2H), 5.68 (bs, 1H, NH), 3.70 (t, 4H), 3.38 (q, 2H), 3.27-3.18 (m, 4H), 2.88 (tt, 1H), 2.42 (t, 4H), 2.34 (t, 2H), 2.05 (d, 2H), 1.90-1.19 (m, 20H).

### Example 11

### N-(7-(2-cyano-3-(pyridin-4-yl)guanidino)heptyl)-N-(3-morpholino propyl)cyclopentanesulfonamide (Compound 1011)

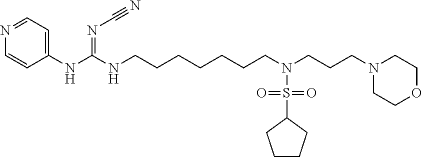

General procedure 5. Starting materials: S-Methyl N-cyano-N′-4-pyridylisothiourea and compound 18. ¹H-NMR (CDCl₃): δ 8.48 (d, 2H), 7.25 (d, 2H), 5.78 (t, 1H, NH), 3.69 (t, 4H), 3.50-3.34 (m, 3H), 3.27-3.18 (m, 4H), 2.41 (t, 4H), 2.34 (t, 2H), 1.98-1.91 (m, 4H), 1.81-1.73 (m, 4H), 1.66-1.55 (m, 6H), 1.36-1.28 (m, 6H).

### Example 12

### N-(3-morpholinopropyl)-N-(8-(3-(pyridin-3-ylmethyl)ureido)octyl)cyclopentanesulfonamide (Compound 1012)

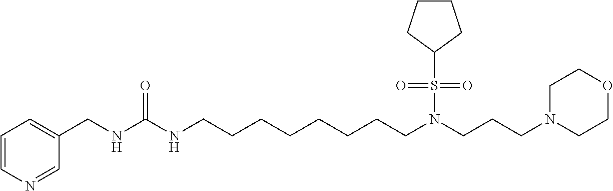

General procedure 6. Starting materials: 3-picolylamine and compound 20. ¹H-NMR (CDCl₃): δ 8.49 (d, 1H), 8.46 (dd, 1H), 7.63 (dt, 1H), 7.22 (dd, 1H), 5.29 (t, 1H, NH), 4.87 (t, 1H, NH), 4.35 (d, 2H), 3.69 (t, 4H), 3.42 (q, 1H), 3.26-3.11 (m, 6H), 2.43 (t, 4H), 2.35 (t, 2H), 1.96-1.89 (m, 4H), 1.80-1.70 (m, 4H), 1.64-1.21 (m, 14H).

### Example 13

### N-(3-morpholinopropyl)-N-(8-(3-(pyridin-3-ylmethyl)ureido)octyl)cyclohexanesulfonamide (Compound 1013)

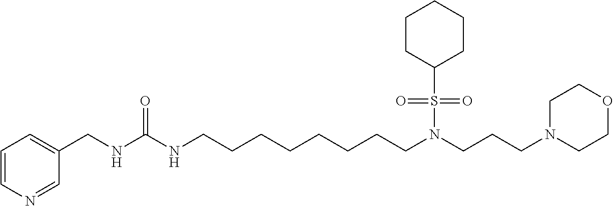

General procedure 6. Starting materials: 3-picolylamine compound 21. ¹H-NMR (CDCl₃): δ 8.51 (d, 1H), 8.47 (dd, 1H), 7.64 (dt, 1H), 7.22 (dd, 1H), 5.15 (t, 1H, NH), 4.74 (t, 1H, NH), 4.37 (d, 2H), 3.69 (t, 4H), 3.26-3.12 (m, 6H), 2.84 (tt, 1H), 2.41 (t, 4H), 2.33 (t, 2H), 2.02 (d, 2H), 1.87-1.17 (m, 20H).

### Example 14

### N-(3-morpholinopropyl)-N-(7-(3-(pyridin-3-ylmethyl)ureido)heptyl) cyclohexanesulfonamide (Compound 1014)

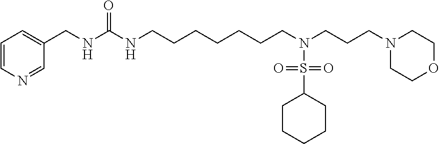

General procedure 6. Starting materials: 3-picolylamine and compound 19. ¹H-NMR (CDCl₃): δ 8.50 (d, 1H), 8.47 (dd, 1H), 7.63 (dt, 1H), 7.22 (dd, 1H), 5.22 (t, 1H, NH), 4.85 (t, 1H, NH), 4.35 (d, 2H), 3.69 (t, 4H), 3.25-3.12 (m, 6H), 0.85 (tt, 1H), 2.41 (t, 4H), 2.33 (t, 2H), 2.05-1.17 (m, 22H).

### Example 15

### N-(3-morpholinopropyl)-N-(7-(3-(pyridin-3-ylmethyl)ureido)heptyl) cyclopentanesulfonamide (Compound 1015)

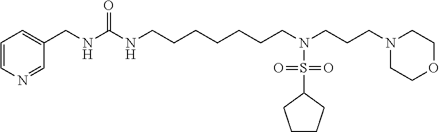

General procedure 6. Starting materials: 3-picolylamine and compound 18. ¹H-NMR (CDCl₃): δ 8.49 (d, 1H), 8.46 (dd, 1H), 7.63 (dt, 1H), 7.22 (dd, 1H), 5.31 (t, 1H, NH), 4.93 (t, 1H, NH), 4.35 (d, 2H), 3.70 (t, 4H), 3.42 (q, 1H), 3.26-3.11 (m, 6H), 2.43 (t, 4H), 2.35 (t, 2H), 1.96-1.89 (m, 4H), 1.81-1.25 (m, 14H).

### Example 16

### N-(8-(3,4-dioxo-2-(pyridin-4-ylamino)cyclobut-1-enylamino)octyl)-N-(3-morpholinopropyl)cyclopentanesulfonamide (Compound 1016)

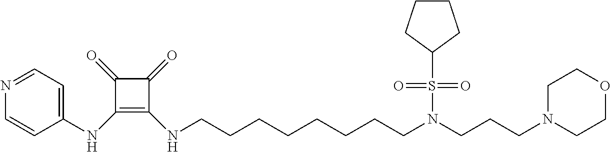

General procedure 7. Starting materials: 3-ethoxy-4-(pyridin-4-ylamino)cyclobut-3-ene-2,3-dione and compound 20. ¹H-NMR (DMSO-d₆): δ 9.89 (1H, NH), 8.41 (d, 2H), 7.80 (bs, 1H, NH), 7.43 (d, 2H), 3.63-3.54 (m, 7H), 3.19-3.11 (m, 4H), 2.32 (t, 4H), 2.26 (t, 2H), 1.92-1.25 (m, 22H).

### Example 17

### N-(8-(3,4-dioxo-2-(pyridin-4-ylamino)cyclobut-1-enylamino)octyl)-N-(3-morpholinopropyl)cyclohexanesulfonamide (Compound 1017)

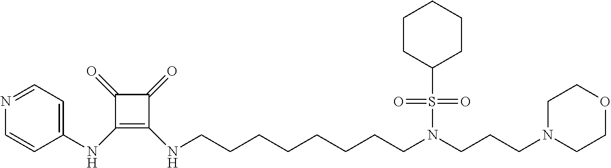

General procedure 7. Starting materials: 3-ethoxy-4-(pyridin-4-ylamino)cyclobut-3-ene-2,3-dione and compound 21. ¹H-NMR (CDCl₃): δ 9.15 (bs, 1H, NH), 8.44 (d, 1H), 7.48 (d, 1H), 7.12 (bs, 1H), 3.79-3.70 (m, 6H), 3.29-3.19 (m, 4H), 2.94 (tt, 1H), 2.41 (t, 4H), 2.34 (t, 2H), 2.08 (d, 2H), 1.91-1.87 (m, 2H), 1.81-1.46 (m, 8H), 1.33-1.20 (m, 12H).

### Example 18

### N-(7-(3,4-dioxo-2-(Pyridin-4-ylamino)cyclobut-1-enylamino)heptyl)-N-(3-morpholinopropyl)cyclohexanesulfonamide (Compound 1018)

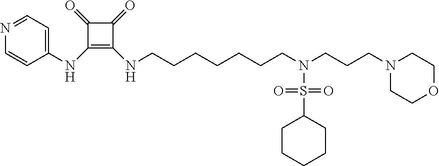

General procedure 7. Starting materials: 3-ethoxy-4-(pyridin-4-ylamino)cyclobut-3-ene-2,3-dione and compound 19. ¹H-NMR (DMSO-d₆): δ 9.89 (br, 1H, NH), 8.40 (d, 2H), 7.80 (bs, 1H, NH), 7.43 (d, 2H), 3.63-3.54 (m, 6H), 3.20-3.11 (m, 4H), 3.07-2.99 (m, 1H), 2.31 (t, 4H), 2.25 (t, 2H), 1.96-1.22 (m, 24H).

### Example 19

### N-(7-(3,4-dioxo-2-(pyridin-4-ylamino)cyclobut-1-enylamino)heptyl)-N-(3-morpholinopropyl)cyclopentanesulfonamide (Compound 1019)

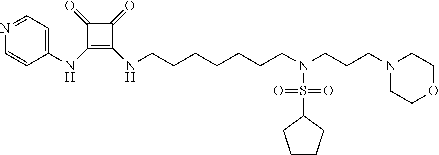

General procedure 7. Starting materials: 3-ethoxy-4-(pyridin-4-ylamino)cyclobut-3-ene-2,3-dione and compound 18. ¹H-NMR (CDCl₃): δ 8.45 (d, 2H), 7.52 (d, 2H), 7.00 (bs, 1H, NH), 3.73-3.68 (m, 6H), 3.52 (m, 1H), 3.31-3.21 (m, 4H), 2.42 (t, 4H), 2.35 (t, 2H), 2.00-1.28 (m, 20H).

### Example 20

### N-(benzyloxy)-N-(8-(2-cyano-3-pyridin-4-yl)guanidino)octyl)methanesulfonamide (Compound 1020)

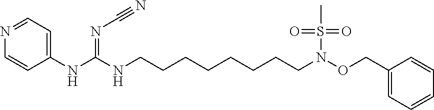

General procedure 5. Starting materials: S-Methyl N-cyano-N′-4-pyridylisothiourea and compound 24. ¹H-NMR (CD₃OD): δ 8.39 (m, 2H), 7.39 (m, 7H), 5.01 (s, 2H), 3.40 (t, 2H), 3.17 (t, 2H), 2.95 (s, 3H), 1.60 (m, 4H), 1.36 (m, 8H).

### Example 21

### N-(Benzyloxy)-N-(8-(3,4-dioxo-2-(pyridin-4-ylamino)cyclobute-1-enylamino)octyl)methansulfonamide (Compound 1021)

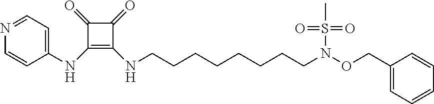

General procedure 7. Starting materials: 3-ethoxy-4-(pyridin-4-ylamino)cyclobut-3-ene-2,3-dione and compound 24. ¹H-NMR (DMSO-d₆): 9.89 (bs, 1H), 8.40 (d, 2H), 7.80 (t, 1H), 7.41 (d, 2H), 7.38 (m, 5H), 4.94 (s, 2H), 3.61 (m, 2H), 3.10 (t, 2H), 3.02 (s, 3H), 1.53 (m, 4H), 1.28 (m, 8H).

### Example 22

### N-(Benzyloxy)-N-(8-(3-pyridin-3-ylmethyl)ureido)octyl)methansulfonamide (Compound 1022)

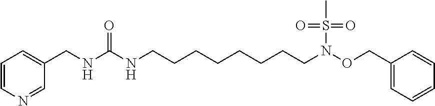

General procedure 6. Starting materials: 3-picolylamine and compound 24. ¹H-NMR (CD₃OD): δ 8.49 (d, 1H), 8.41 (dd, 1H), 7.79 (dt, 1H), 7.39 (m, 6H), 5.01 (s, 2H), 4.36 (s, 2H), 3.16 (m, 4H), 2.96 (s, 3H), 1.53 (m, 4H), 1.32 (m, 8H).

### Example 23

### N-(8-(N-Benzyloxy)methylsulfonamido)octyl-3-(pyridin-3-yl)acrylamide (Compound 1023)

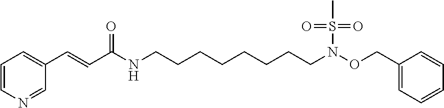

General procedure 10. Starting materials: (E)-3-(pyridin-3-yl)acrylic acid and compound 24. ¹H-NMR (CD₃OD): δ 8.72 (d, 1H), 8.52 (dd, 1H), 8.05 (dt, 1H), 7.56 (d, 1H), 7.47 (m, 1H), 7.38 (m, 5H), 6.75 (d, 1H), 5.00 (s, 2H), 3.33 (t, 2H), 3.16 (t, 2H), 2.95 (s, 3H), 1.57 (m, 4H), 1.34 (m, 8H).

### Example 24

### N-(benzyloxy)-N-(8-(2-cyano-3-Pyridin-4-yl)guanidino)octyl)propane-2-sulfonamide (Compound 1024)

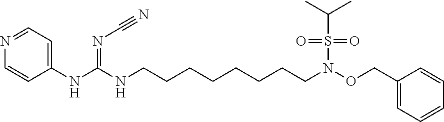

General procedure 5. Starting materials: S-Methyl N-cyano-N′-4-pyridylisothiourea and compound 27. ¹H-NMR (CD₃OD): δ 8.39 (m, 2H), 7.38 (m, 7H), 4.99 (s, 2H), 3.60 (m, 1H), 3.40 (t, 2H), 3.31 (t, 2H), 1.62 (m, 4H), 1.41 (d, 6H), 1.38 (m, 8H).

### Example 25

### N-(Benzyloxy)-N-(8-(3,4-dioxo-2-(pyridin-4-ylamino)cyclobute-1-enylamino)octyl)propane-2-sulfonamide (Compound 1025)

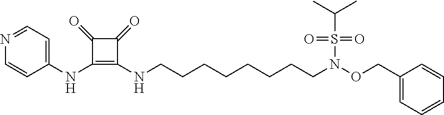

General procedure 7. Starting materials: 3-ethoxy-4-(pyridin-4-ylamino)cyclobut-3-ene-2,3-dione and compound 27. ¹H-NMR (DMSO-d₆): 9.87 (bs, 1H), 8.40 (d, 2H), 7.80 (bs, 1H), 7.42 (d, 2H), 7.37 (m, 5H), 4.92 (s, 2H), 3.62 (m, 3H), 3.23 (t, 2H), 1.55 (m, 4H), 1.30 (m, 10H).

### Example 26

### N-(Benzyloxy)-N-(8-(3-pyridin-3-ylmethyl)ureido)octyl)propane-2-sulfonamide (Compound 1026)

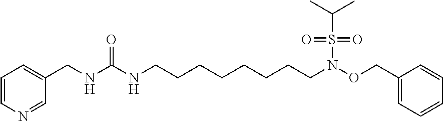

General procedure 6. Starting materials: 3-picolylamine and compound 27. ¹H-NMR (CD₃OD): δ 8.49 (d, 1H), 8.41 (dd, 1H), 7.78 (dt, 1H), 7.38 (m, 6H), 4.99 (s, 2H), 4.36 (s, 2H), 3.60 (m, 1H), 3.31 (t, 2H), 3.14 (t, 2H), 1.60 (m, 2H), 1.49 (m, 2H), 1.41 (d, 6H), 1.33 (m, 8H).

### Example 27

### N-(8-(N-Benzyloxy)propan-2-ylsulfonamido)octyl)-3-(pyridin-3-yl)acrylamide (Compound 1027)

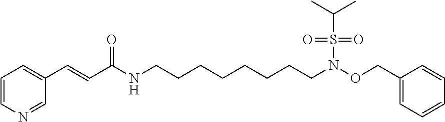

General procedure 10. Starting materials: (E)-3-(pyridin-3-yl)acrylic acid and compound 27. ¹H-NMR (CD₃OD): δ 8.71 (d, 1H), 8.52 (dd, 1H), 8.05 (dt, 1H), 7.56 (d, 1H), 7.48 (m, 1H), 7.38 (m, 5H), 6.75 (d, 1H), 4.99 (s, 2H), 3.59 (m, 1H), 3.31 (m, 4H), 1.59 (m, 4H), 1.40 (d, 6H), 1.36 (m, 8H).

### Example 28

### N-(Benzyloxy)-N-(8-(3-pyridin-4-ylureido)octyl)propane-2-sulfonamide (Compound 1028)

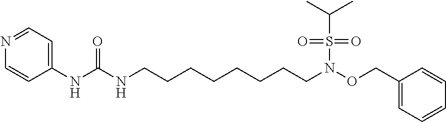

General procedure 11. Starting materials: 4-aminopyridine and compound 27. ¹H-NMR (CD₃OD): δ 8.27 (m, 2H), 7.45 (m, 2H), 7.38 (m, 5H), 4.99 (s, 2H), 3.60 (m, 1H), 3.31 (t, 2H), 3.22 (t, 2H), 1.58 (m, 4H), 1.41 (d, 6H), 1.36 (m, 8H).

### Example 29

### N-(Benzyloxy)-N-(8-(3-pyridin-4-ylthioureido)octyl)propane-2-sulfonamide (Compound 1029)

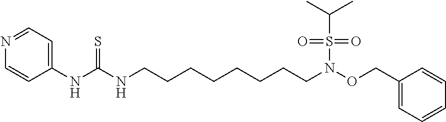

General procedure 12. Starting materials: 4-aminopyridine and compound 27. ¹H-NMR (CD₃OD): δ 8.34 (m, 2H), 7.72 (m, 2H), 7.38 (m, 5H), 5.00 (s, 2H), 3.60 (m, 3H), 3.32 (m, 2H), 1.65 (m, 4H), 1.41 (d, 6H), 1.38 (m, 8H).

### Example 30

### N-(8-(2-cyano-3-(pyridin-4-yl)guanidino)octyl)-N-(3-morpholinopropyl)methanesulfonamide (Compound 1030)

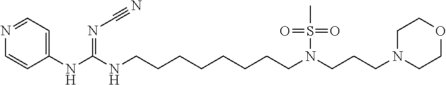

General procedure 5. Starting materials: S-Methyl N-cyano-N′-4-pyridylisothiourea and compound 30. ¹H-NMR (CD₃OD): δ 8.40 (d, 2H), 7.35 (d, 2H), 3.71 (m, 4H), 3.40 (t, 2H), 3.22 (m, 4H), 2.88 (s, 3H), 2.48 (m, 4H), 2.41 (t, 2H), 1.82 (m, 2H), 1.63 (m, 4H), 1.40 (m, 8H).

### Example 31

### N-(8-(3,4-dioxo-2-(pyridin-4-ylamino)cyclobute-1-enylamino)octyl)-N-(3-morpholinopropyl)methanesulfonamide (Compound 1031)

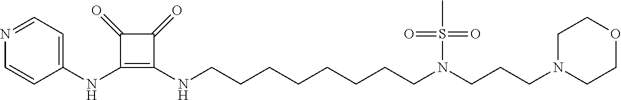

General procedure 7. Starting materials: 3-ethoxy-4-(pyridin-4-ylamino)cyclobut-3-ene-2,3-dione and compound 30. ¹H-NMR (CD₃OD): δ 8.37 (m, 2H), 7.53 (m, 2H), 3.70 (m, 6H), 3.20 (m, 4H), 2.87 (s, 3H), 2.47 (t, 4H), 2.40 (t, 2H), 1.81 (m, 2H), 1.66 (m, 4H), 1.40 (m, 8H).

### Example 32

### N-(3-Morpholinopropyl)-N-(8-(3-pyridin-3-ylmethyl)ureido)octyl)methanesulfonamide (Compound 1032)

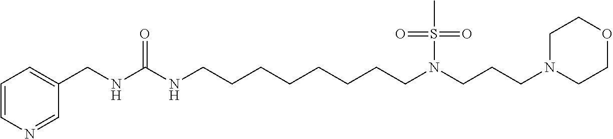

General procedure 6. Starting materials: 3-picolylamine and compound 30. ¹H-NMR (CD₃OD): δ 8.49 (d, 1H), 8.43 (dd, 1H), 7.79 (dt, 1H), 7.41 (m, 1H), 4.36 (s, 2H), 3.71 (t, 4H), 3.19 (m, 6H), 2.88 (s, 3H), 2.48 (t, 4H), 2.41 (t, 2H), 1.82 (m, 2H), 1.63 (m, 2H), 1.50 (m, 2H), 1.36 (m, 8H).

### Example 33

### N-(8-(N-(3-morpholinopropyl)methylsulfonamido)octyl)-3-(pyridin-3-yl)acrylamide (Compound 1033)

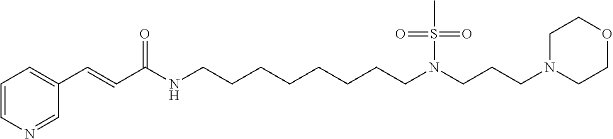

General procedure 10. Starting materials: (E)-3-(pyridin-3-yl)acrylic acid and compound 30. ¹H-NMR (CD₃OD): δ 8.73 (d, 1H), 8.53 (dd, 1H), 8.06 (dt, 1H), 7.57 (d, 1H), 7.48 (m, 1H), 6.75 (d, 1H), 3.70 (m, 4H), 3.32 (m, 2H), 3.21 (m, 4H), 2.88 (s, 3H), 2.47 (m, 4H), 2.41 (t, 2H), 1.82 (m, 2H), 1.60 (m, 4H), 1.34 (m, 8H).

### Example 34

### N-(3-Morpholinopropyl)-N-(8-(3-Pyridin-4-ylthioureido)octyl)methanesulfonamide (Compound 1034)

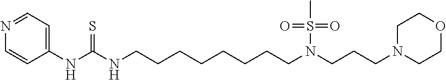

General procedure 12. Starting materials: 4-aminopyridine and compound 30. ¹H-NMR (CD₃OD): δ 8.35 (m, 2H), 7.73 (m, 2H), 3.70 (m, 4H), 3.60 (t, 2H), 3.21 (m, 4H), 2.48 (m, 4H), 2.41 (t, 2H), 1.82 (m, 2H), 1.65 (m, 4H), 1.40 (m, 8H).

### Example 35

### N-(8-(2-cyano-3-(pyridin-4-yl)guanidino)octyl)-N-(3-morpholinopropyl)benzenesulfonamide (Compound 1035)

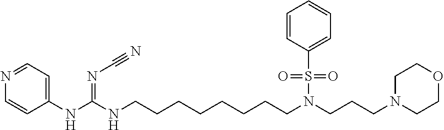

General procedure 5. Starting materials: S-Methyl N-cyano-N′-4-pyridylisothiourea and compound 33. ¹H-NMR (CD₃OD): δ 8.39 (m, 2H), 7.84 (m, 2H), 7.62 (m, 3H), 7.35 (d, 2H), 3.69 (m, 4H), 3.40 (t, 2H), 3.18 (m, 4H), 2.43 (m, 4H), 2.35 (t, 2H), 1.75 (m, 2H), 1.64 (m, 2H), 1.54 (m, 2H), 1.34 (m, 8H).

### Example 36

### N-(8-(3,4-dioxo-2-(pyridin-4-ylamino)cyclobute-1-enylamino)octyl)-N-(3-morpholinopropyl)benzenesulfonamide (Compound 1036)

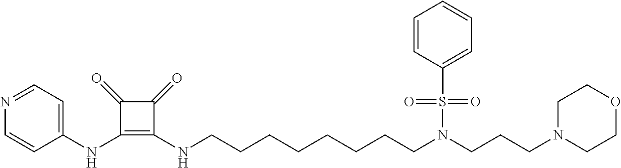

General procedure 7. Starting materials: 3-ethoxy-4-(pyridin-4-ylamino)cyclobut-3-ene-2,3-dione and compound 33. ¹H-NMR (DMSO-d₆): δ 9.88 (bs, H), 8.40 (m, 2H), 7.77 (m, 3H), 7.62 (m, 3H), 7.43 (m, 2H), 3.60 (m, 2H), 3.53 (t, 4H), 3.08 (t, 4H), 2.25 (m, 4H), 2.20 (t, 2H), 1.57 (m, 4H), 1.43 (m, 2H), 1.25 (m, 8H).

### Example 37

### N-(3-Morpholinopropyl)-N-(8-(3-pyridin-3-ylmethyl)ureido)octyl)benzenesulfonamide (Compound 1037)

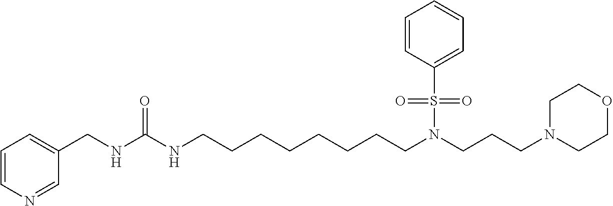

General procedure 6. Starting materials: 3-picolylamine and compound 33. ¹H-NMR (CD₃OD): δ 8.49 (d, 1H), 8.43 (dd, 1H), 7.82 (m, 3H), 7.62 (m, 3H), 7.40 (m, 1H), 4.37 (s, 2H), 3.69 (m, 4H), 3.16 (m, 6H), 2.48 (t, 4H), 2.35 (t, 2H), 1.75 (m, 2H), 1.51 (m, 4H), 1.30 (m, 8H).

### Example 38

### N-(8-(N-(3-morpholinopropyl)phenylsulfonamido)octyl)-3-(pyridin-3-yl)acrylamide (Compound 1038)

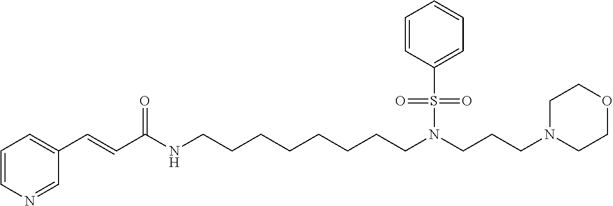

General procedure 10. Starting materials: (E)-3-(pyridin-3-yl)acrylic acid and compound 33. ¹H-NMR (CD₃OD): δ 8.73 (d, 1H), 8.53 (dd, 1H), 8.06 (dt, 1H), 7.84 (m, 2H), 7.62 (m, 4H), 7.49 (m, 1H), 6.76 (d, 1H), 3.69 (m, 4H), 3.32 (m, 2H), 3.19 (m, 4H), 2.43 (m, 4H), 2.36 (t, 2H), 1.75 (m, 2H), 1.56 (m, 4H), 1.33 (m, 8H).

### Example 39

### N-(3-Morpholinopropyl)-N-(8-(3-pyridin-4-ylureido)octyl)benzenesulfonamide (Compound 1039)

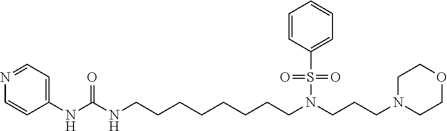

General procedure 11. Starting materials: 4-aminopyridine and compound 33. MS [M+H]⁺=532.3, [M−H]⁻=530.3, [M−H+HCOOH]⁻=576.4.

### Example 40

### N-(3-Morpholinopropyl)-N-(8-(3-pyridin-4-ylureido)octyl)benzenesulfonamide (Compound 1040)

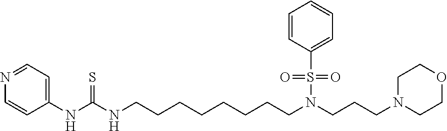

General procedure 12. Starting materials: 4-aminopyridine and compound 33. ¹H-NMR (CD₃OD): δ 8.35 (m, 2H), 7.84 (m, 2H), 7.73 (m, 2H), 7.63 (m, 3H), 3.69 (t, 4H), 3.60 (t, 2H), 3.20 (m, 4H), 2.44 (t, 4H), 2.36 (t, 2H), 1.76 (m, 2H), 1.67 (m, 2H), 1.55 (m, 2H), 1.36 (m, 8H).

### Example 41

### 1-(7-Cyclohexyl(3-morpholinopropyl)amino)heptyl)-3-(pyridin-4-yl)thiourea oxalate (Compound 1041)

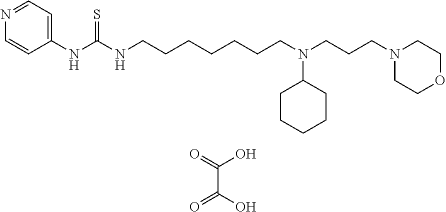

General procedure 12. Starting materials: 4-aminopyridine and compound 10. ¹H-NMR (400 MHz, DMSO-d₆): δ 10.44 (bs, 1H), 8.80 (bs, 1H), 8.37 (d, 2H), 7.77-7.65 (m, 2H), 3.66-3.54 (m, 4H), 3.53-3.42 (m, 2H), 3.22 (t, 1H), 3.14-2.96 (m, 4H), 2.58-2.36 (m, 6H), 2.01-1.88 (m, 2H), 1.88-1.71 (m, 4H), 1.70-1.50 (m, 5H), 1.50-1.20 (m, 10H), 1.20-1.04 (m, 1H).

### Example 42

### 1-(7-Cyclohexyl(3-morpholinopropyl)amino)heptyl)-3-(Pyridin-4-yl)urea oxalate (Compound 1042)

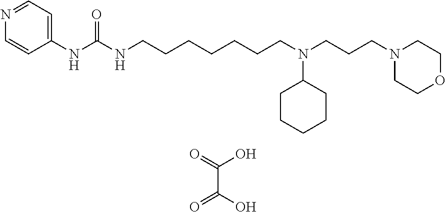

General procedure 11. Starting materials: 4-aminopyridine and compound 10. ¹H-NMR (400 MHz, DMSO-d₆): δ 10.36 (bs, 1H), 8.37 (d, 2H), 7.61 (d, 2H), 7.45 (bs, 1H), 3.68-3.60 (m, 4H), 3.22 (t, 1H), 3.14-2.98 (m, 6H), 2.36-2.65 (m, 6H), 1.99-1.82 (m, 4H), 1.82-1.73 (m, 2H), 1.68-1.54 (m, 3H), 1.50-1.20 (m, 12H), 1.18-1.04 (m, 1H).

### Example 43

### (E)-N-(7-Cyclohexyl(3-morpholinopropyl)amino)heptyl)-3-(pyridin-3-yl)acrylamide (Compound 1043)

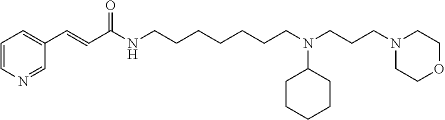

General procedure 10. Starting materials: (E)-3-(pyridin-3-yl)acrylic acid and compound 10. ¹H-NMR (CDCl₃): δ 8.73 (d, 1H), 8.55 (dd, 1H), 7.78 (dt, 1H), 7.61 (d, 1H), 7.30 (m, 1H), 6.50 (d, 1H), 5.94 (bs, 1H), 3.70 (t, 4H), 3.38 (q, 2H), 2.42 (m, 9H), 2.32 (t, 2H), 1.76 (m, 4H), 1.7-0.95 (m, 18H).

### Example 44

### N-(6-(2-Cyano-3-pyridin-4-yl)guanidino)hexyl)-N-(3-morpholinopropyl)cyclopentanesulfonamide (Compound 1044)

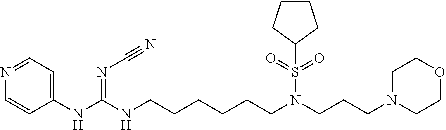

General procedure 5. Starting materials: S-Methyl N-cyano-N′-4-pyridylisothiourea and compound 35. ¹H-NMR (CD₃OD): δ 8.40 (m, 2H), 7.35 (m, 2H), 3.70 (m, 5H), 3.42 (t, 2H), 3.28 (m, 4H), 2.49 (m, 4H), 2.41 (t, 2H), 1.95 (m, 4H), 1.79 (m, 4H), 1.65 (m, 6H), 1.42 (m, 4H).

### Example 45

### N-(6-(3,4-Dioxo-2-(pyridin-4-ylamino)cyclobut-1-enylamino)hexyl-N-(3-morpholinopropyl)cyclopentanesulfonamide (Compound 1045)

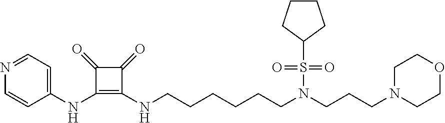

General procedure 7. Starting materials: 3-ethoxy-4-(pyridin-4-ylamino)cyclobut-3-ene-2,3-dione and compound 35. ¹H-NMR (CD₃OD): δ 8.36 (m, 2H), 7.54 (m, 2H), 3.69 (m, 8H), 3.25 (m, 3H), 2.46 (m, 4H), 2.38 (t, 2H), 2.05-1.25 (m, 18H).

### Example 46

### N-(3-morpholinopropyl)-N-(6-(3-pyridin-3-ylmethyl)ureido)hexyl)cyclopentanesulfonamide (Compound 1046)

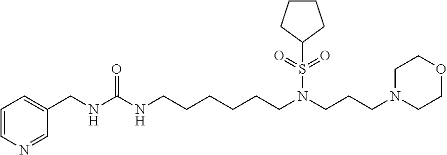

General procedure 6. Starting materials: 3-picolylamine and compound 35. ¹H-NMR (CD₃OD): δ 8.49 (d, 1H), 8.43 (dd, 1H), 7.80 (m, 1H), 7.42 (m, 1H), 4.37 (s, 2H), 3.70 (m, 6H), 3.25 (m, 3H), 3.15 (t, 2H), 2.53 (m, 4H), 2.45 (t, 2H), 1.96 (m, 4H), 1.80 (m, 4H), 1.65 (m, 4H), 1.51 (m, 2H), 1.37 (m, 4H).

### Example 47

### (E)-N-(6-(N-(3-morpholinopropyl)cyclopentanesulfonamido)hexyl)-3-(Pyridin-3-yl)acrylamide (Compound 1047)

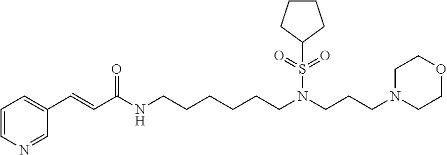

General procedure 10. Starting materials: (E)-3-(pyridin-3-yl)acrylic acid and compound 35. ¹H-NMR (CD₃OD): δ 8.73 (d, 1H), 8.53 (dd, 1H), 8.06 (dt, 1H), 7.57 (d, 1H), 7.49 (m, 1H), 6.74 (d, 1H), 3.69 (m, 6H), 3.28 (m, 5H), 2.46 (m, 4H), 2.39 (t, 2H), 1.95 (m, 4H), 1.77 (m, 4H), 1.62 (m, 6H), 1.41 (m, 4H).

### Example 48

### N-(3-morpholinopropyl)-N-(6-(3-pyridin-4-ylureido)hexyl)cyclopentanesulfonamide (Compound 1048)

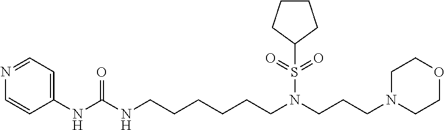

General procedure 11. Starting materials: 4-aminopyridine and compound 35. MS [M+H]⁺=496.3, [M−H+HCOOH]⁻=540.3.

### Example 49

### N-(3-morpholinopropyl)-N-(6-(3-pyridin-4-ylthioureido)hexyl)cyclopentanesulfonamide (Compound 1049)

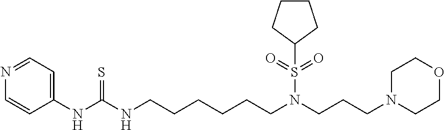

General procedure 12. Starting materials: 4-aminopyridine and compound 35. ¹H-NMR (CD₃OD): δ 8.35 (m, 2H), 7.73 (m, 2H), 3.66 (m, 8H), 3.28 (m, 3H), 2.48 (m, 4H), 2.41 (m, 2H), 2.05-1.25 (m, 18H).

### Example 50

### N-(6-(2-Cyano-3-pyridin-4-yl)guanidino)hexyl)-N-(3-morpholinopropyl)cyclohexanesulfonamide (Compound 1050)

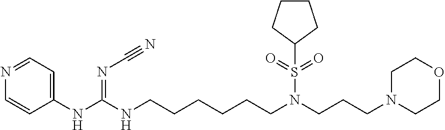

General procedure 5. Starting materials: S-Methyl N-cyano-N′-4-pyridylisothiourea and compound 37. ¹H-NMR (CD₃OD): δ 8.40 (d, 2H), 7.35 (d, 2H), 3.70 (m, 4H), 3.41 (t, 2H), 3.28 (m, 4H), 3.05 (m, 1H), 2.47 (m, 4H), 2.39 (t, 2H), 2.07 (m, 2H), 1.95-1.0 (m, 18H).

### Example 51

### N-(6-(3,4-Dioxo-2-(pyridin-4-ylamino)cyclobut-1-enylamino)hexyl-N-(3-morpholinopropyl)cyclohexanesulfonamide (Compound 1051)

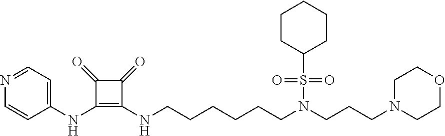

General procedure 7. Starting materials: 3-ethoxy-4-(pyridin-4-ylamino)cyclobut-3-ene-2,3-dione and compound 37. ¹H-NMR (DMSO-d₆): δ 9.89 (bs, 1H), 8.40 (d, 2H), 7.81 (bs, 1H), 7.43 (d, 2H), 3.60 (m, 2H), 3.55 (t, 4H), 3.16 (m, 4H), 3.04 (m, 1H), 2.31 (t, 4H), 2.24 (t, 2H), 1.94 (m, 2H), 1.8-1.0 (m, 18H).

### Example 52

### N-(3-morpholinopropyl)-N-(6-(3-pyridin-3-ylmethyl)ureido)hexyl)cyclohexanesulfonamide (Compound 1052)

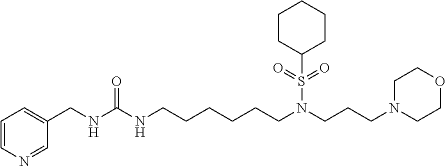

General procedure 6. Starting materials: 3-picolylamine and compound 37. ¹H-NMR (CD₃OD): δ 8.49 (d, 1H), 8.43 (dd, 1H), 7.79 (dt, 1H), 7.41 (m, 1H), 4.37 (s, 2H), 3.71 (t, 4H), 3.26 (m, 4H), 3.15 (t, 2H), 3.05 (m, 1H), 2.47 (m, 4H), 2.39 (t, 2H), 2.07 (m, 2H), 1.95-1.05 (m, 18H).

### Example 53

### (E)-N-(6-(N-(3-morpholinopropyl)cyclohexanesulfonamido)hexyl)-3-(Pyridin-3-yl)acrylamide (Compound 1053)

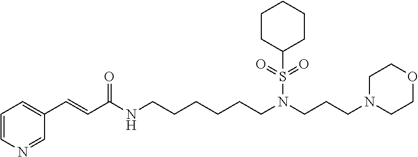

General procedure 10. Starting materials: (E)-3-(pyridin-3-yl)acrylic acid and compound 37. ¹H-NMR (CD₃OD): δ 8.73 (d, 1H), 8.53 (dd, 1H), 8.07 (dt, 1H), 7.57 (d, 1H), 7.49 (m, 1H), 6.76 (d, 1H), 3.70 (m, 4H), 3.28 (m, 6H), 3.05 (m, 1H), 2.47 (m, 4H), 2.39 (t, 2H), 2.07 (m, 2H), 1.95-1.05 (m, 18H).

### Example 54

### N-(3-morpholinopropyl)-N-(6-(3-pyridin-4-ylthioureido)hexyl)cyclohexanesulfonamide (Compound 1054)

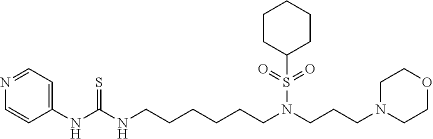

General procedure 12. Starting materials: 4-aminopyridine and compound 37. ¹H-NMR (CD₃OD): δ 8.35 (m, 2H), 7.73 (m, 2H), 3.70 (m, 4H), 3.62 (t, 2H), 3.28 (m, 4H), 3.05 (m, 1H), 2.48 (m, 4H), 2.40 (t, 2H), 2.07 (m, 2H), 1.95-1.0 (m, 18H).

### Example 55

### N-(7-(2-Cyano-3-pyridin-4-yl)guanidino)heptyl)-1-phenyl-N-(tetrahydro-2H-pyran-2-yloxy)methanesulfonamide (Compound 1055)

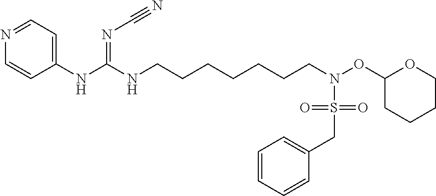

General procedure 5. Starting materials: S-Methyl N-cyano-N′-4-pyridylisothiourea and compound 40. ¹H-NMR (CD₃OD): δ 8.38 (d, 2H), 7.38 (m, 7H), 5.08 (m, 1H), 4.50 (q, 4H), 3.95 (m, 1H), 3.60 (m, 1H), 3.42 (m, 3H), 3.14 (m, 1H), 1.85-1.2 (m, 16H).

### Example 56

### N-(7-(3,4-Dioxo-2-(pyridin-4-ylamino)cyclobut-1-enylamino)heptyl)-1-Phenyl-N-(tetrahydro-2H-pyran-2-yloxy)methanesulfonamide (Compound 1056)

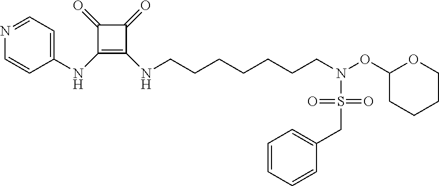

General procedure 7. Starting materials: 3-ethoxy-4-(pyridin-4-ylamino)cyclobut-3-ene-2,3-dione and compound 40. ¹H-NMR (CD₃OD): δ 8.35 (m, 2H), 7.52 (m, 2H), 7.44 (m, 2H), 7.38 (m, 3H), 5.07 (m, 1H), 4.49 (q, 2H), 3.94 (m, 1H), 3.72 (t, 2H), 3.60 (m, 1H), 3.42 (m, 1H), 3.14 (m, 1H), 1.85-1.2 (m, 16H).

### Example 57

### 1-Phenyl-N-(7-(3-(pyridin-3-ylmethyl)ureido)heptyl)-N-(tetrahydro-2H-pyran-2-yloxy)methanesulfonamide (Compound 1057)

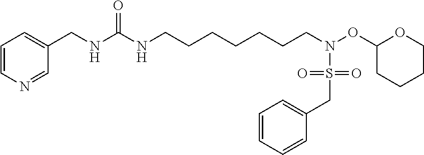

General procedure 6. Starting materials: 3-picolylamine and compound 40. ¹H-NMR (CD₃OD): δ 8.49 (d, 1H), 8.43 (dd, 1H), 7.79 (dt, 1H), 7.43 (m, 6H), 5.08 (m, 1H), 4.50 (q, 2H), 4.37 (s, 2H), 3.94 (m, 1H), 3.59 (m, 1H), 3.42 (m, 2H), 3.14 (m, 2H), 1.85-1.2 (m, 16H).

### Example 58

### (E)-N-(7-(1-phenyl-N-(tetrahydro-2H-pyran-2-yloxy)methylsulfonamido)heptyl)-3-(pyridin-3-yl)acrylamide (Compound 1058)

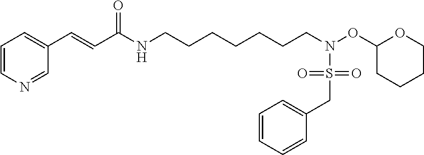

General procedure 10. Starting materials: (E)-3-(pyridin-3-yl)acrylic acid and compound 40. ¹H-NMR (CD₃OD): δ 8.71 (d, 1H), 8.52 (dd, 1H), 8.04 (dt, 1H), 7.56 (d, 1H), 7.41 (m, 6H), 6.74 (d, 1H), 5.08 (m, 1H), 4.49 (q, 2H), 3.94 (m, 1H), 3.59 (m, 1H), 3.42 (m, 2H), 3.30 (m, 1H), 3.14 (m, 1H), 1.9-1.2 (m, 16H).

### Example 59

### 1-phenyl-N-(7-(3-pyridin-4-ylthioureido)heptyl)-N-(tetrahydro-2H-pyran-2-yloxy)methanesulfonamide (Compound 1059)

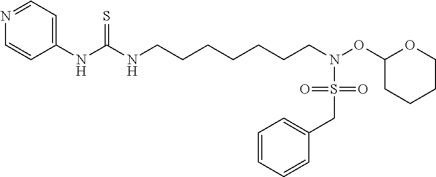

General procedure 12. Starting materials: 4-aminopyridine and compound 40. ¹H-NMR (CD₃OD): δ 8.34 (m, 2H), 7.73 (m, 2H), 7.46 (m, 2H), 7.39 (m, 3H), 5.10 (m, 1H), 4.50 (q, 2H), 3.95 (m, 1H), 3.60 (m, 3H), 3.45 (m, 1H), 3.15 (m, 1H), 1.85-1.25 (m, 16H).

### Example 60

### N-(7-(2-cyano-3-(pyridin-4-yl)guanidino)heptyl)-N-(cyclohexylmethoxy)ethanesulfonamide (Compound 1060)

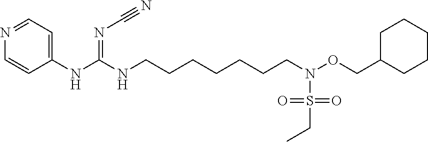

General procedure 5. Starting materials: S-Methyl N-cyano-N′-4-pyridylisothiourea and compound 43. ¹H-NMR (CDCl₃): δ 8.43 (m, 2H), 7.23 (m, 2H), 6.00 (bs, 1H), 3.80 (d, 2H), 3.37 (m, 2H), 3.17 (t, 2H), 3.08 (q, 2H), 1.68 (m, 10H), 1.41 (t, 3H), 1.35 (m, 6H), 1.19 (m, 3H), 0.97 (m, 2H).

### Example 61

### N-(cyclohexylmethoxy)-N-(7-(3,4-dioxo-2-(pyridin-4-ylamino)cyclobut-1-enylamino)heptyl)ethanesulfonamide (Compound 1061)

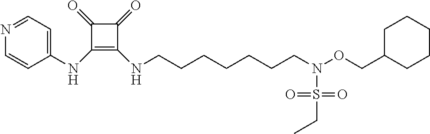

General procedure 7. Starting materials: 3-ethoxy-4-(pyridin-4-ylamino)cyclobut-3-ene-2,3-dione and compound 43. ¹H-NMR (DMSO-d₆): δ 9.89 (bs, 1H), 8.41 (d, 2H), 7.80 (t, 1H), 7.43 (d, 2H), 3.74 (d, 2H), 3.61 (m, 2H), 3.18 (q, 2H), 3.14 (m, 2H), 1.61 (m, 10H), 1.34 (m, 6H), 1.28 (t, 3H), 1.14 (m, 3H), 0.96 (m, 2H).

### Example 62

### N-(cyclohexylmethoxy)-N-(7-(3-(pyridin-3-ylmethyl)ureido)heptyl)ethanesulfonamide (Compound 1062)

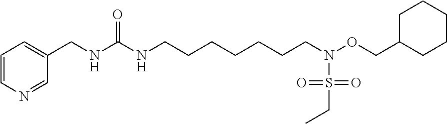

General procedure 6. Starting materials: 3-picolylamine and compound 43. ¹H-NMR (CDCl₃): δ 8.45 (m, 2H), 7.60 (dt, 1H), 7.20 (m, 1H), 5.48 (t, 1H), 5.08 (t, 1H), 4.32 (d, 2H), 3.79 (d, 2H), 3.10 (m, 6H), 1.68 (m, 8H), 1.40 (t, 3H), 1.35 (m, 11H), 0.97 (m, 2H).

### Example 63

### (E)-N-(7-(N-(cyclohexylmethoxy)ethylsulfonamido)heptyl)-3-(pyridin-3-yl)acrylamide (Compound 1063)

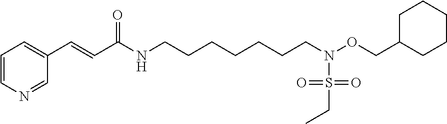

General procedure 10. Starting materials: (E)-3-(pyridin-3-yl)acrylic acid and compound 43. ¹H-NMR (CDCl₃): δ 8.72 (d, 1H), 8.54 (dd, 1H), 7.78 (dt, 1H), 7.59 (d, 1H), 7.29 (m, 1H), 6.52 (d, 1H), 6.14 (t, 1H), 3.81 (d, 2H), 3.37 (q, 2H), 3.18 (t, 2H), 3.08 (q, 2H), 1.63 (m, 10H), 1.43 (t, 3H), 1.35 (m, 6H), 1.18 (m, 3H), 0.97 (m, 2H).

### Example 64

### N-(cyclohexylmethoxy)-N-(7-(3-(pyridin-4-yl)ureido)heptyl)ethanesulfonamide (Compound 1064)

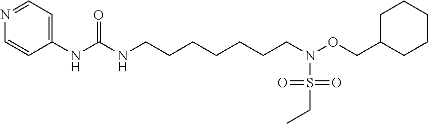

General procedure 11. Starting materials: 4-aminopyridine and compound 43. ¹H-NMR (CDCl₃): δ 8.34 (m, 2H), 8.18 (s, 1H), 7.39 (m, 2H), 5.55 (t, 1H), 3.81 (d, 2H), 3.21 (m, 4H), 3.14 (q, 2H), 1.69 (m, 10H), 1.46 (t, 3H), 1.28 (m, 9H), 0.98 (m, 2H).

### Example 65

### N-(cyclohexylmethoxy)-N-(7-(3-(pyridin-4-yl)thioureido)heptyl)ethanesulfonamide (Compound 1065)

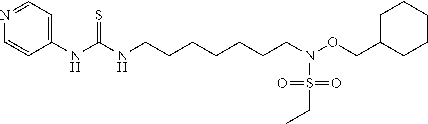

General procedure 12. Starting materials: 4-aminopyridine and compound 43. ¹H-NMR (CDCl₃): δ 8.53 (bs, 1H), 8.48 (d, 2H), 7.44 (d, 2H), 6.82 (bs 1H), 3.82 (d, 2H), 3.63 (m, 2H), 3.20 (t, 2H), 3.12 (q, 2H), 1.66 (m, 10H), 1.44 (t, 3H), 1.36 (m, 6H), 1.20 (m, 3H), 0.97 (m, 2H).

### Example 66

### N-(7-(2-cyano-3-(pyridin-4-yl)guanidino)heptyl)-N-(cyclohexyloxy)-4-fluorobenzenesulfonamide (Compound 1066)

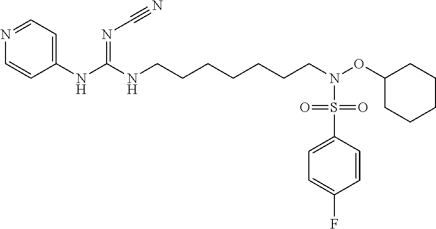

General procedure 5. Starting materials: S-Methyl N-cyano-N′-4-pyridylisothiourea and compound 46. ¹H-NMR (CDCl₃): δ 8.46 (m, 2H), 7.86 (m, 2H), 7.22 (m, 4H), 5.86 (bs, 1H), 4.14 (m, 1H), 3.35 (m, 2H), 2.83 (bs, 2H), 2.06 (m, 2H), 1.72 (m, 2H), 1.58 (m, 4H), 1.28 (m, 12H).

### Example 67

### N-(cyclohexyloxy)-N-(7-(3,4-dioxo-2-(pyridin-4-ylamino)cyclobut-1-enylamino)heptyl)-4-fluorobenzenesulfonamide (Compound 1067)

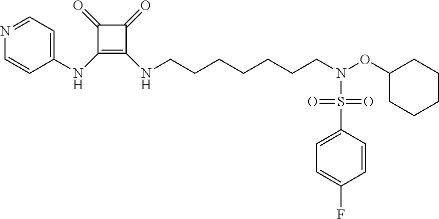

General procedure 7. Starting materials: 3-ethoxy-4-(pyridin-4-ylamino)cyclobut-3-ene-2,3-dione and compound 46. ¹H-NMR (DMSO-d₆): δ 9.91 (bs, 1H), 8.41 (d, 2H), 7.92 (m, 2H), 7.79 (t, 1H), 7.52 (m, 2H), 7.43 (d, 2H), 4.05 (m, 1H), 3.60 (m, 2H), 2.8 (bs, 2H), 1.98 (m, 2H), 1.67 (m, 2H), 1.51 (m, 4H), 1.27 (m, 12H).

### Example 68

### N-(cyclohexyloxy)-4-fluoro-N-(7-(3-(pyridin-3-ylmethyl)ureido)heptyl) benzenesulfonamide (Compound 1068)

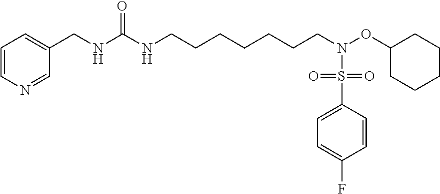

General procedure 6. Starting materials: 3-picolylamine and compound 46. ¹H-NMR (CDCl₃): δ 8.44 (m, 2H), 7.85 (m, 2H), 7.60 (dt, 1H), 7.21 (m, 3H), 5.37 (t, 1H), 4.98 (t, 1H), 4.32 (d, 2H), 4.12 (m, 1H), 3.35 (t, 2H), 3.11 (q, 2H), 2.05 (m, 2H), 1.73 (m, 2H), 1.54 (m, 4H), 1.42 (m, 2H), 1.25 (m, 10H).

### Example 69

### (E)-N-(7-(N-(cyclohexyloxy)-4-fluorophenylsulfonamido)heptyl)-3-(pyridin-3-yl)acrylamide (Compound 1069)

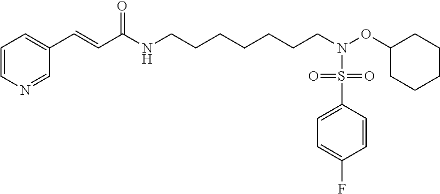

General procedure 10. Starting materials: (E)-3-(pyridin-3-yl)acrylic acid and compound 46. ¹H-NMR (CDCl₃): δ 8.74 (d, 1H), 8.55 (dd, 1H), 7.89 (m, 2H), 7.79 (dt, 1H), 7.61 (d, 1H), 7.30 (m, 1H), 7.23 (t, 2H), 6.53 (d, 1H), 6.11 (t, 1H), 4.16 (m, 1H), 3.38 (q, 2H), 2.85 (bs, 2H), 2.08 (m, 2H), 1.76 (m, 2H), 1.57 (m, 4H), 1.45-1.0 (m, 12H).

### Example 70

### N-(cyclohexyloxy)-4-fluoro-N-(7-(3-(pyridin-4-yl)thioureido)heptyl) benzenesulfonamide (Compound 1070)

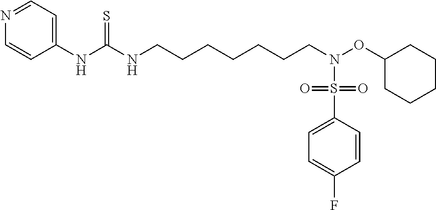

General procedure 12. Starting materials: 4-aminopyridine and compound 46. ¹H-NMR CDCl₃): δ 8.50 (m, 2H), 7.88 (m, 2H), 7.35 (m, 2H), 7.23 (t, 2H), 6.74 (t, 1H), 4.14 (m, 1H), 3.63 (m, 2H), 2.8 (bs, 2H), 2.06 (m, 2H), 1.75 (m, 2H), 1.58 (m, 4H), 1.4-1.0 (m, 12H).

### Example 71

### N-(8-(2-cyano-3-(pyridin-4-yl)guanidino)octyl)-N-(3-morpholinopropyl)benzamide (Compound 1071)

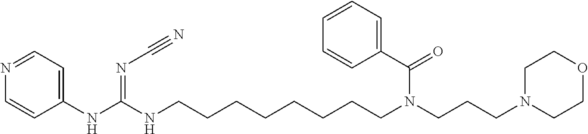

General procedure 5. Starting materials: S-Methyl N-cyano-N′-4-pyridylisothiourea and compound 51. ¹H-NMR (CD₃OD): δ 8.39 (d, 2H), 7.46 (m, 3H), 7.36 (m, 4H), 3.72 (m, 2H), 3.54 (m, 4H), 3.30 (m, 2H), 2.51 (m, 4H), 2.19 (m, 4H), 2.0-1.0 (m, 14H).

### Example 72

### N-(8-(3,4-dioxo-2-(pyridin-4-ylamino)cyclobut-1-enylamino)octyl)-N-(3-morpholinopropyl)benzamide (Compound 1072)

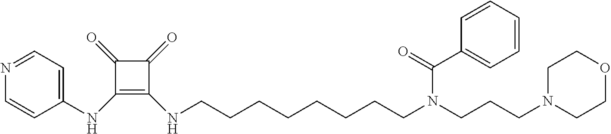

General procedure 7. Starting materials: 3-ethoxy-4-(pyridin-4-ylamino)cyclobut-3-ene-2,3-dione and compound 51. ¹H-NMR (DMSO-d₆): δ 9.89 (bs, 1H), 8.41 (d, 2H), 7.80 (bs, 1H), 7.41 (m, 5H), 7.31 (m, 2H), 3.58 (m, 4H), 3.17 (m, 4H), 2.35 (m, 4H), 2.06 (m, 4H), 1.8-0.95 (m, 14H).

### Example 73

### N-(3-morpholinopropyl)-N-(8-(3-(Pyridin-3-ylmethyl)ureido)octyl)benzamide (Compound 1073)

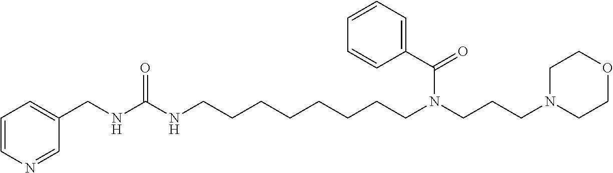

General procedure 6. Starting materials: 3-picolylamine and compound 51. ¹H-NMR (DMSO-d₆): δ 8.45 (d, 1H), 8.41 (dd, 1H), 7.62 (dt, 1H), 7.41 (m, 3H), 7.32 (m, 3H), 6.36 (t, 1H), 5.97 (bs, 1H), 4.20 (d, 2H), 4.13 (q, 2H), 3.57 (bs, 4H), 3.16 (bs, 2H), 2.97 (bs, 2H), 2.37 (m, 4H), 2.07 (m, 2H), 1.8-0.9 (m, 14).

### Example 74

### (E)-N-(3-morpholinopropyl)-N-(8-(3-(pyridin-3-yl)acrylamido)octyl)benzamide (Compound 1074)

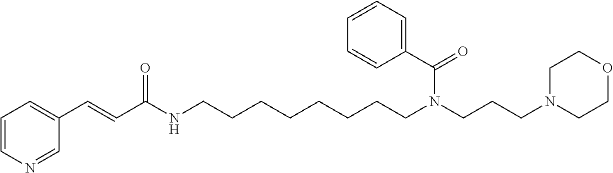

General procedure 10. Starting materials: (E)-3-(pyridin-3-yl)acrylic acid and compound 51. ¹H-NMR (CD₃OD): δ 8.72 (d, 1H), 8.52 (dd, 1H), 8.05 (dt, 1H), 7.57 (d, 1H), 7.46 (m, 4H), 7.37 (m, 2H), 6.73 (d, 1H), 3.72 (bs, 2H), 3.53 (bs, 4H), 3.31 (m, 2H), 2.51 (m, 4H), 2.20 (m, 4H), 1.92 (m, 2H), 1.8-1.0 (m, 12H).

### Example 75

### N-(3-morpholinopropyl)-N-(8-(3-(pyridin-4-yl)thioureido)octyl)benzamide (Compound 1075)

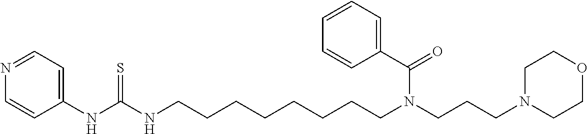

General procedure 12. Starting materials: 4-aminopyridine and compound 51. MS [M+H]⁺=512.4, [M−H]⁻=510.4.

### Example 76

### N-(8-(2-cyano-3-(pyridin-3-yl)guanidino)octyl)-N-(3-morpholinopropyl)benzamide (Compound 1076)

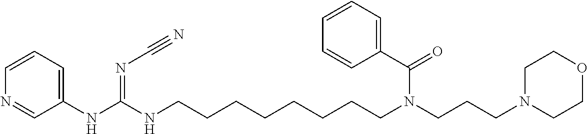

General procedure 5. Starting materials: S-Methyl N-cyano-N′-3-pyridylisothiourea (see e.g. WO/2009/086835) and compound 51. MS [M+H]⁺=520.4, [M−H]⁻=518.4.

### Example 77

### 3-cyclohexyl-1-(3-morpholinopropyl)-1-(8-(3-(pyridin-4-yl)thioureido)octyl)urea (Compound 1077)

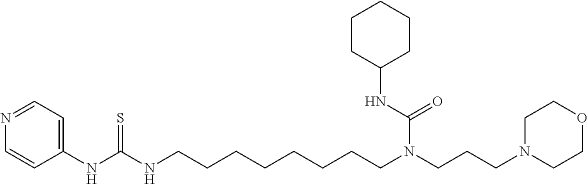

General procedure 12. Starting materials: 4-aminopyridine and compound 53. MS [M+H]⁺=533.4, [M−H]=531.4.

### Example 78

### 3-(8-(benzyloxy(ethyl)amino)octylamino)-4-(pyridin-4-ylamino)cyclobut-3-ene-1,2-dione (Compound 1078)

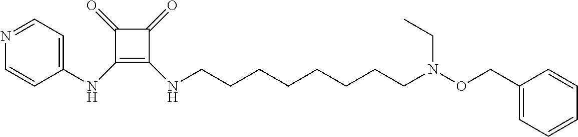

General procedure 7. Starting materials: 3-ethoxy-4-(pyridin-4-ylamino)cyclobut-3-ene-2,3-dione and compound 56. ¹H-NMR (400 MHz, CDCl₃, HMDSO): δ 9.77 (bs, 1H), 8.41 (d, 2H), 7.98 (bs, 1H), 7.40 (d, 2H), 7.34-7.21 (m, 5H), 4.67 (s, 2H), 3.74 (t, 2H), 2.73 (q, 2H), 2.64 (t, 2H), 1.69-1.50 (m, 4H), 1.39-1.23 (m, 8H), 1.13 (t, 3H).

### Example 79

### N-(3-morpholinopropyl)-N-(7-(3-(pyridin-4-yl)thioureido)heptyl)cyclohexanesulfonamide oxalate (Compound 1079)

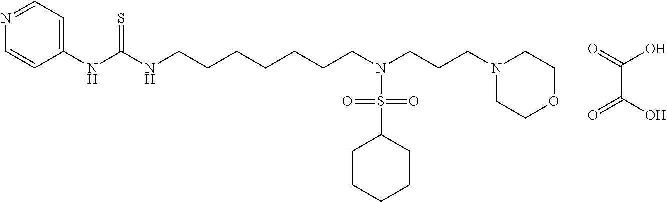

General procedure 12. Starting materials: 4-aminopyridine and compound 19. ¹H-NMR (400 MHz, CDCl₃, HMDSO): δ 10.43 (bs, 1H), 8.75 (bs, 1H), 8.41-8.37 (m, 2H), 7.70-7.77 (m, 2H), 3.72 (bs, 4H), 3.47 (q, 2H), 3.21 (t, 2H), 3.14 (t, 2H), 3.07 (m, 1H), 2.91 (bs, 4H), 2.78 (t, 2H), 2.00-1.89 (m, 2H), 1.87-1.71 (m, 4H), 1.65-1.45 (m, 5H), 1.41-1.18 (m, 10H), 1.04-1.18 (m, 1H).

### Example 80

### 1-(8-(benzyloxy(ethyl)amino)octyl)-3-(pyridin-4-yl)thiourea oxalate (Compound 1080)

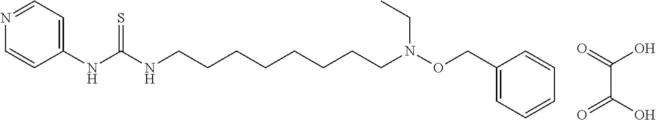

General procedure 12. Starting materials: 4-aminopyridine and compound 56. ¹H-NMR (400 MHz, CDCl₃, HMDSO): δ 10.52 (bs, 1H), 8.81 (bs, 1H), 8.46-8.40 (m, 2H), 7.81 (d, 2H), 7.36-7.26 (m, 5H), 4.62 (s, 2H), 3.48 (q, 2H), 2.68 (q, 2H), 2.61 (t, 2H), 1.56 (m, 2H), 1.49 (m, 2H), 1.34-1.24 (m, 8H), 1.05 (t, 3H).

### Example 81

### 1-(8-(benzyloxy(ethyl)amino)octyl)-3-(pyridin-3-ylmethyl)urea oxalate (Compound 1081)

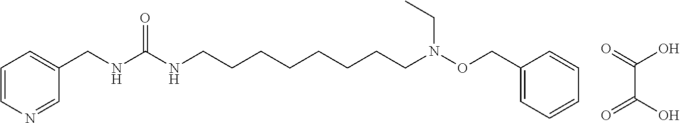

General procedure 6. Starting materials: 3-picolylamine and compound 56. ¹H-NMR (400 MHz, DMSO-d₆, HMDSO): δ 8.54-8.38 (m, 2H), 7.68 (d, 1H), 7.41-7.24 (m, 6H), 6.35 (bs, 1H), 5.96 (bs, 1H), 4.62 (s, 2H), 4.21 (d, 2H), 2.98 (m, 2H), 2.68 (q, 2H), 2.60 (t, 2H), 1.54-1.42 (m, 2H), 1.42-1.17 (m, 10H), 1.05 (t, 3H).

### Example 82

### 1-(8-(benzyloxy(ethyl)amino)octyl)-3-(pyridin-4-yl)urea (Compound 1082)

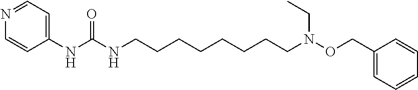

General procedure 11. Starting materials: 4-aminopyridine and compound 56. ¹H-NMR (400 MHz, CDCl₃, HMDSO): δ 8.36 (d, 2H), 7.7-6.8 (bs, 1H), 7.39-7.25 (m, 7H), 5.09 (t, 1H), 4.70 (s, 2H), 3.23 (q, 2H), 2.76 (q, 2H), 2.67 (t, 2H), 1.58 (m, 2H), 1.49 (m, 2H), 1.36-1.22 (m, 8H), 1.16 (t, 3H).

### Example 83

### N-(3-morpholinopropyl)-N-(7-(3-(pyridin-4-yl)ureido)heptyl)cyclohexanesulfonamide (Compound 1083)

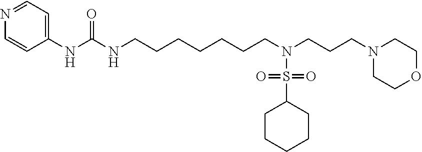

General procedure 11. Starting materials: 4-aminopyridine and compound 19. ¹H-NMR (400 MHz, CDCl₃, HMDSO): δ 8.38-8.34 (m, 2H), 7.80 (s, 1H), 7.41-7.37 (m, 2H), 5.48 (t, 1H), 3.75-3.66 (m, 4H), 3.30-3.17 (m, 6H), 2.93 (m, 1H), 2.44-2.37 (m, 4H), 2.34 (t, 2H), 2.13-2.04 (m, 2H), 1.94-1.85 (m, 2H), 1.82-1.67 (m, 3H), 1.63-1.43 (m, 6H), 1.36-1.13 (m, 9H).

### Example 84

### 1-(8-(benzyloxy(ethyl)amino)octyl)-2-cyano-3-(pyridin-4-yl)guanidine (Compound 1084)

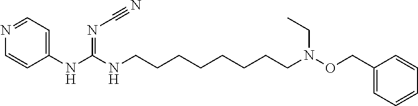

General procedure 5. Starting materials: S-Methyl N-cyano-N′-4-pyridylisothiourea and compound 56. ¹H-NMR (400 MHz, DMSO₆, HMDSO): δ ˜9-6.5 (bs, 1H), 8.43 (d, 2H), 8.13 (bs, 1H), 7.37-7.25 (m, 7H), 4.61 (s, 2H), 3.28 (q, 2H), 2.68 (q, 2H), 2.60 (t, 2H), 1.58-1.3 (m, 4H), 1.33-1.22 (m, 8H), 1.05 (t, 3H).

### Example 85

### 1-(8-(benzyl(ethoxy)amino)octyl)-2-cyano-3-(pyridin-4-yl)guanidine (Compound 1085)

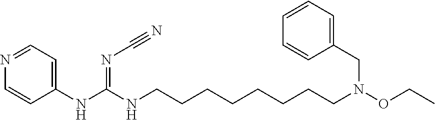

General procedure 5. Starting materials: S-Methyl N-cyano-N′-4-pyridylisothiourea and compound 59. ¹H-NMR (400 MHz, DMSO₆, HMDSO): δ 9.33 (bs, 1H), 8.39 (d, 2H), 7.81 (t, 1H), 7.34-7.16 (m, 7H), 3.75 (s, 2H), 3.44 (q, 2H), 3.25 (q, 2H), 2.58 (t, 2H), 1.56-1.43 (m, 4H), 1.33-1.18 (m, 8H), 0.90 (t, 3H).

### Example 86

### 2-Cyano-1-(8-(ethyl(2-morpholinoethoxy)amino)octyl)-3-(pyridin-4-yl)guanidine oxalate (Compound 1086)

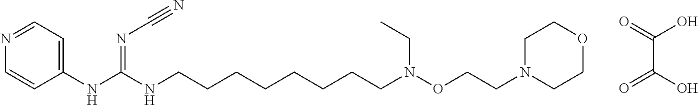

General procedure 5. Starting materials: S-Methyl N-cyano-N′-4-pyridylisothiourea and compound 62. ¹H-NMR (400 MHz, DMSO₆, HMDSO): δ 8.41 (d, 2H), 8.12 (bs, 1H), 7.27 (d, 2H), 3.87 (t, 2H), 3.76-3.69 (m, 4H), 3.28 (q, 2H), 3.02 (t, 2H), 2.99-2.92 (m, 4H), 2.68 (q, 2H), 2.61 (t, 2H), 1.57-141 (m, 4H), 1.33-1.24 (m, 8H), 1.04 (t, 3H).

### Example 87

### 2-Cyano-1-(8-(3-morpholinopropylamino)octyl)-3-(pyridin-4-yl)guanidine (Compound 1087)

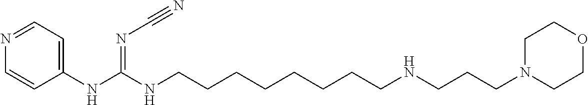

General procedure 5. Starting materials: S-Methyl N-cyano-N′-4-pyridylisothiourea and compound 63. ¹H-NMR (CD₃OD): δ 8.38 (m, 2H), 7.33 (d, 2H), 3.70 (t, 4H), 3.40 (t, 2H), 2.62 (m, 4H), 2.48 (t, 4H), 2.42 (t, 2H), 1.73 (m, 2H), 1.65 (m, 2H), 1.54 (m, 2H), 1.39 (m, 8H).

### Example 88

### 2-Cyano-1-(8-((dimethylphosphoryl)(3-morpholinopropyl)amino)octyl)-3-(pyridin-4-yl)guanidine (Compound 1088)

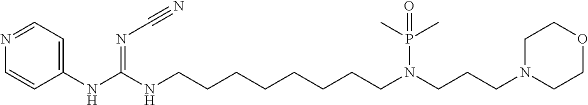

General procedure 5. Starting materials: S-Methyl N-cyano-N′-4-pyridylisothiourea and compound 65. ¹H-NMR (CD₃OD): δ 8.39 (m, 2H), 7.35 (d, 2H), 3.70 (t, 4H), 3.40 (t, 2H), 3.02 (m, 4H), 2.48 (t, 4H), 2.37 (t, 2H), 1.75 (m, 2H), 1.65 (m, 2H), 1.57 (m, 2H), 1.53 (d, 6H), 1.38 (m, 8H).

### Example 89

### N-(8-(3,4-dioxo-2-(pyridin-4-ylamino)cyclobut-1-enylamino)octyl)-P,P-dimethyl-N-(3-morpholinopropyl)phosphinic amide (Compound 1089)

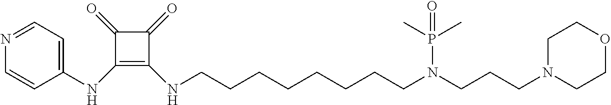

General procedure 7. Starting materials: 3-ethoxy-4-(pyridin-4-ylamino)cyclobut-3-ene-2,3-dione and compound 65. ¹H-NMR (CD₃OD): δ 8.37 (m, 2H), 7.54 (m, 2H), 3.71 (m, 6H), 3.02 (m, 4H), 2.47 (t, 4H), 2.37 (t, 2H), 1.72 (m, 4H), 1.55 (m, 2H), 1.53 (d, 6H), 1.39 (m, 8H).

### Example 90

### 1-(8-((dimethylphosphoryl)(3-morpholinopropyl)amino)octyl)-3-(pyridin-3-ylmethyl)urea (Compound 1090)

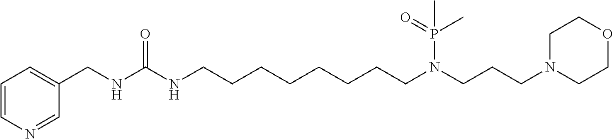

General procedure 6. Starting materials: 3-picolylamine and compound 65. ¹H-NMR (CD₃OD): δ 8.49 (d, 1H), 8.43 (dd, 1H), 7.79 (dt, 1H), 7.41 (m, 1H), 4.37 (s, 2H), 3.71 (t, 4H), 3.14 (t, 2H), 3.03 (m, 4H), 2.48 (m, 4H), 2.38 (t, 2H), 1.75 (m, 2H), 1.57 (m, 2H), 1.53 (d, 6H), 1.51 (m, 2H), 1.35 (m, 8H).

### Example 91

### (E)-N-(8-((dimethylphosphoryl)(3-morpholinopropyl)amino)octyl)-3-(pyridin-3-yl)acrylamide (Compound 1091)

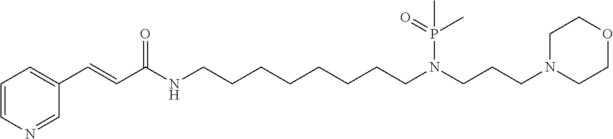

General procedure 10. Starting materials: (E)-3-(pyridin-3-yl)acrylic acid and compound 65. ¹H-NMR (CD₃OD): δ 8.73 (d, 1H), 8.53 (dd, 1H), 8.06 (dt, 1H), 7.55 (d, 1H), 7.49 (m, 1H), 6.75 (d, 1H), 3.70 (t, 4H), 3.32 (m, 2H), 3.02 (m, 4H), 2.47 (m, 4H), 2.37 (t, 2H), 1.75 (m, 2H), 1.58 (m, 4H), 1.53 (d, 6H), 1.40 (m, 8H).

### Example 92

### 1-(8-((dimethylphosphoryl)(3-morpholinopropyl)amino)octyl)-3-(pyridin-4-yl)thiourea (Compound 1092)

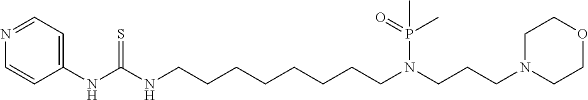

General procedure 12. Starting materials: 4-aminopyridine and compound 65. ¹H-NMR (CD₃OD): δ 8.35 (m, 2H), 7.73 (d, 2H), 3.71 (t, 4H), 3.61 (t, 2H), 3.04 (m, 4H), 2.48 (t, 4H), 2.38 (t, 2H), 1.75 (m, 2H), 1.67 (m, 2H), 1.58 (m, 2H), 1.54 (d, 6H), 1.40 (m, 8H).

### Example 93

### 1-(8-(2-cyano-3-(pyridin-4-yl)guanidino)octyl)-3-cyclohexyl-1-(morpholinopropyl)urea (Compound 1093)

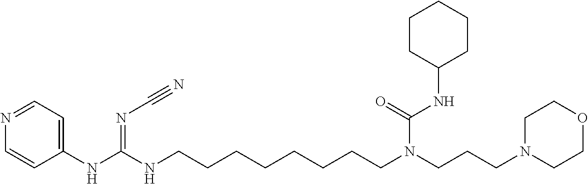

Compound 87 (16 mg, 0.04 mmol) was dissolved in DCM, cyclohexylisocyanate (0.005 ml, 0.043 mmol) and NEt₃ (0.006 ml, 0.043 mmol) were added with stirring and left at rt for 7 days, concentrated and purified by chromatography (chloroform:methanol:NH₃ (25% aq.) 98:2:0.2 to 95:5:1) to afford compound 93. ¹H-NMR (CD₃OD): δ 8.39 (m, 2H), 7.35 (m, 2H), 3.72 (m, 4H), 3.53 (m, 1H), 3.40 (t, 2H), 3.26 (m, 4H), 2.47 (m, 4H), 2.36 (t, 2H), 1.95-1.1 (m, 24H).

### Example 94

### 1-(8-(2-cyano-3-(pyridin-4-yl)guanidino)octyl)-1-(3-morpholinopropyl)-3-Phenylthiourea (Compound 1094)

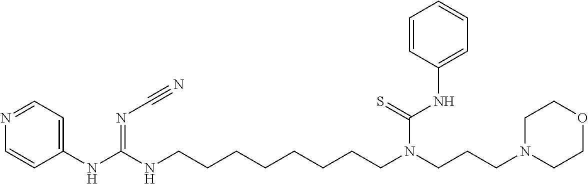

Compound 87 (17 mg, 0.04 mmol) was dissolved in DCM, phenyl isothiocyanate (0.009 ml, 0.045 mmol) and NEt₃ (0.006 ml, 0.045 mmol) were added with stirring and left at rt for 7 days, concentrated and purified by chromatography (chloroform:methanol:NH₃ (25% aq.) 98:2:0.2 to 95:5:1) to afford compound 94. ¹H-NMR (CD₃OD): δ 8.39 (m, 2H), 7.30 (m, 7H), 3.78 (m, 4H), 3.53 (t, 4H), 3.40 (t, 2H), 2.46 (m, 6H), 1.95 (m, 2H), 1.78 (m, 2H), 1.65 (m, 2H), 1.42 (m, 8H).

### Example 95

### N-(8-(2-cyano-3-(pyridin-4-yl)guanidino)octyl)-N-(3-morpholinopropyl)hydrazinecarboxamide (Compound 1095)

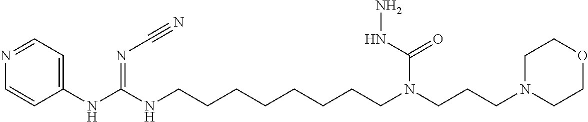

General procedure 5. Starting materials: S-Methyl N-cyano-N′-4-pyridylisothiourea and compound 66. MS [M+H]⁺=474.4.

### Example 96

### N-(8-(3,4-dioxo-2-(pyridin-4-ylamino)cyclobut-1-enylamino)octyl)-N-(3-morpholinopropyl)hydrazinecarboxamide (Compound 1096)

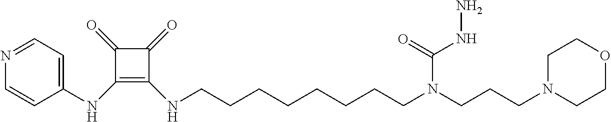

General procedure 7. Starting materials: 3-ethoxy-4-(pyridin-4-ylamino)cyclobut-3-ene-2,3-dione and compound 67. MS [M+H]⁺=502.4, [M−H]⁻=500.3.

### Example 97

### N-(3-morpholinopropyl)-N-(8-(3-(pyridin-3-ylmethyl)ureido)octyl)hydrazinecarboxamide (Compound 1097)

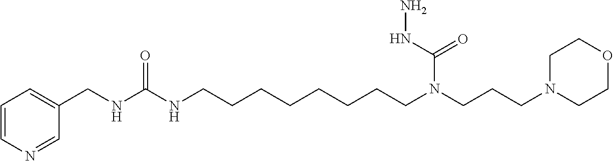

General procedure 6. Starting materials: 3-picolylamine and compound 66. ¹H-NMR (CD₃OD): δ 8.49 (d, 1H), 8.43 (dd, 1H), 7.79 (dt, 1H), 7.41 (m, 1H), 4.42 (s, 2H), 3.64 (t, 4H), 3.30 (m, 4H), 3.17 (t, 2H), 2.42 (m, 4H), 2.36 (t, 2H), 1.79 (m, 2H), 1.56 (m, 4H), 1.35 (m, 8H).

### Example 98

### N-(7-(2-cyano-3-(pyridin-4-yl)guanidino)heptyl)-N-(2-fluoroethyl)cyclohexansylfonamide (Compound 1098)

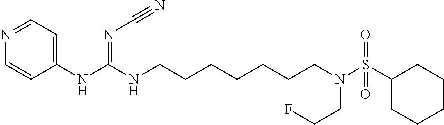

General procedure 5. Starting materials: S-Methyl N-cyano-N′-4-pyridylisothiourea and compound 69. ¹H-NMR (CD₃OD): δ 8.39 (d, 2H), 7.35 (d, 2H), 4.55 (dt, 2H), 3.52 (dt, 2H), 3.41 (t, 2H), 3.31 (t, 2H), 3.10 (m, 1H), 2.10 (m, 2H), 1.88 (m, 2H), 1.75-1.1 (m, 16H).

### Example 99

### N-(7-(3,4-dioxo-2-(pyridin-4-ylamino)cyclobut-1-enylamino)heptyl)-N-(2-fluoroethyl)cyclohexanesulfonamide (Compound 1099)

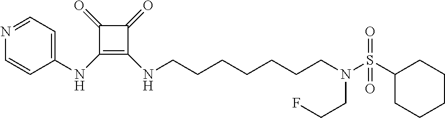

General procedure 7. Starting materials: 3-ethoxy-4-(pyridin-4-ylamino)cyclobut-3-ene-2,3-dione and compound 69. ¹H-NMR (DMSO-d₆): δ 9.88 (bs, 1H), 8.41 (d, 2H), 7.80 (t, 1H), 7.43 (d, 2H), 4.51 (dt, 2H), 3.61 (q, 2H), 3.49 (dt, 2H), 3.20 (t, 2H), 3.11 (m, 1H), 1.95 (m, 2H), 1.77 (m, 2H), 1.65-1.05 (m, 16H).

### Example 100

### N-(2-fluoroethyl)-N-(7-(3-(pyridin-3-ylmethyl)ureido)heptyl) cyclohexanesulfonamide (Compound 1100)

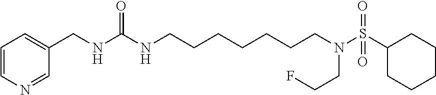

General procedure 6. Starting materials: 3-picolylamine and compound 69. ¹H-NMR (CD₃OD): δ 8.49 (d, 1H), 8.43 (dd, 1H), 7.79 (dt, 1H), 7.42 (m, 1H), 4.56 (dt, 2H), 4.38 (s, 2H), 3.58 (dt, 2H), 3.31 (t, 2H), 3.13 (m, 3H), 2.10 (m, 2H), 1.89 (m, 2H), 1.75-1.1 (m, 16H).

### Example 101

### (E)-N-(7-(N-(2-fluoroethyl)cyclohexanesulfonamido)heptyl)-3-(pyridin-3-yl)acrylamide (Compound 1101)

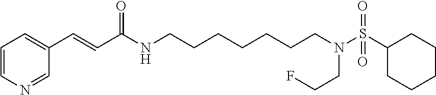

General procedure 10. Starting materials: (E)-3-(pyridin-3-yl)acrylic acid and compound 69. ¹H-NMR (CD₃OD): δ 8.73 (d, 1H), 8.53 (dd, 1H), 8.06 (dt, 1H), 7.55 (d, 1H), 7.49 (m, 1H), 6.75 (d, 1H), 4.55 (dt, 2H), 3.57 (dt, 2H), 3.30 (m, 4H), 3.10 (m, 1H), 2.09 (m, 2H), 1.87 (m, 2H), 1.75-1.1 (m, 16H).

### Example 102

### N-(2-fluoroethyl)-N-(7-(3-pyridin-4-ylthioureido)heptyl)cyclohexanesulfonamide (Compound 1102)

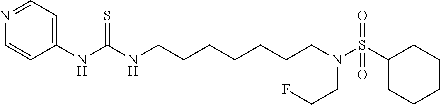

General procedure 12. Starting materials: 4-aminopyridine and compound 69. ¹H-NMR (CD₃OD): δ 8.35 (m, 2H), 7.72 (m, 2H), 4.55 (dt, 2H), 3.61 (m, 3H), 3.53 (t, 1H), 3.31 (t, 2H), 3.10 (m, 1H), 2.09 (m, 2H), 1.88 (m, 2H), 1.75-1.1 (m, 16H).

### Example 103

### N-(7-(2-cyano-3-(pyridin-4-yl)guanidino)octyl)-N-(2-fluoroethyl)cyclohexansulfonamide (Compound 1103)

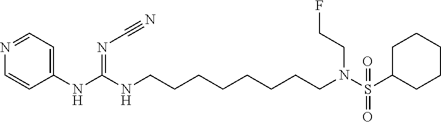

General procedure 5. Starting materials: S-Methyl N-cyano-N′-4-pyridylisothiourea and compound 71. ¹H-NMR (CD₃OD): δ 8.40 (d, 2H), 7.36 (d, 2H), 4.56 (dt, 2H), 3.57 (dt, 2H), 3.41 (t, 2H), 3.31 (t, 2H), 3.10 (m, 1H), 2.10 (m, 2H), 1.89 (m, 2H), 1.75-1.1 (m, 18H).

### Example 104

### N-(7-(3,4-dioxo-2-(pyridin-4-ylamino)cyclobut-1-enylamino)octyl)-N-(2-fluoroethyl)cyclohexanesulfonamide (Compound 1104)

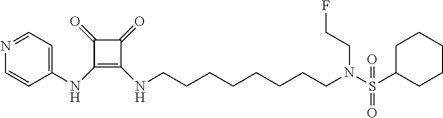

General procedure 7. Starting materials: 3-ethoxy-4-(pyridin-4-ylamino)cyclobut-3-ene-2,3-dione and compound 71. ¹H-NMR (CD₃OD): δ 8.36 (d, 2H), 7.53 (d, 2H), 4.55 (dt, 2H), 3.73 (t, 2H), 3.57 (dt, 2H), 3.30 (t, 2H), 3.10 (m, 1H), 2.09 (m, 2H), 1.86 (m, 2H), 1.8-1.1 (m, 18H).

### Example 105

### (E)-N-(7-(N-(2-fluoroethyl)cyclohexanesulfonamido)octyl)-3-(pyridin-3-yl)acrylamide (Compound 1105)

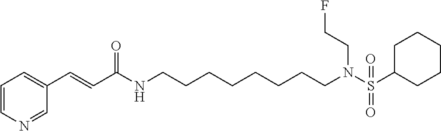

General procedure 10. Starting materials: (E)-3-(pyridin-3-yl)acrylic acid and compound 71. ¹H-NMR (CD₃OD): δ 8.73 (d, 1H), 8.53 (dd, 1H), 8.06 (dt, 1H), 7.55 (d, 1H), 7.49 (m, 1H), 6.75 (d, 1H), 4.55 (dt, 2H), 3.57 (dt, 2H), 3.27 (m, 4H), 3.10 (m, 1H), 2.09 (m, 2H), 1.88 (m, 2H), 1.75-1.05 (m, 18H).

### Example 106

### N-(2-fluoroethyl)-N-(7-(3-pyridin-4-ylthioureido)octyl)cyclohexanesulfonamide (Compound 1106)

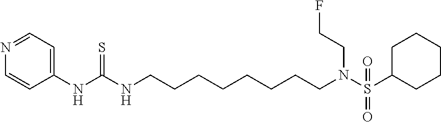

General procedure 12. Starting materials: 4-aminopyridine and compound 71. ¹H-NMR (CD₃OD): δ 8.35 (m, 2H), 7.73 (m, 2H), 4.56 (dt, 2H), 3.61 (m, 3H), 3.53 (t, 1H), 3.31 (t, 2H), 3.10 (m, 1H), 2.09 (m, 2H), 1.88 (m, 2H), 1.75-1.05 (m, 18H).

### Example 107

### 2-cyano-1-(8-(cyclohexylmethoxyamino)octyl)-3-(pyridin-4-yl)guanidine (Compound 1107)

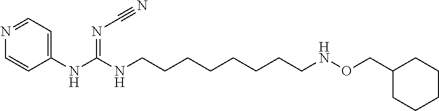

General procedure 5. Starting materials: S-Methyl N-cyano-N′-4-pyridylisothiourea and compound 75. ¹H-NMR (CD₃OD): δ 8.40 (d, 2H), 7.36 (d, 2H), 3.48 (d, 2H), 3.41 (t, 2H), 2.86 (t, 2H), 1.85-1.1 (m, 21H), 0.97 (m, 2H).

### Example 108

### N-(8-(2-cyano-3-(pyridin-4-yl)guanidino)octyl)-N-(cyclohexylmethyloxy)-2,2,2-trifluoromethanesulfonamide (Compound 1108)

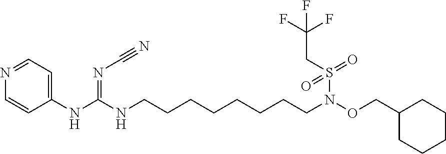

Compound 1107 (11 mg, 0.03 mmol) was dissolved in DCM, 2,2,2-trifluoroethanesulfonyl chloride (0.004 ml, 0.032 mmol) and NEt₃ (0.005 ml, 0.032 mmol) were added with stirring and left at rt overnight, concentrated and purified by chromatography (chloroform:methanol:NH₃ (25% aq.) 98:2:0.2 to 96:4:0.4) to afford compound 1108.

¹H-NMR (CD₃OD): δ 8.40 (d, 2H), 7.35 (d, 2H), 4.23 (q, 2H), 3.89 (d, 2H), 3.41 (t, 2H), 3.28 (t, 2H), 1.85-0.9 (m, 23H).

### Example 109

### 1-(8-(2-cyano-3-(pyridin-4-yl)guanidino)octyl)-3-cyclohexyl-1-(cyclohexylmethoxy)thiourea (Compound 1109)

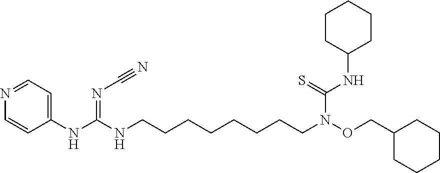

Compound 1107 (9 mg, 0.02 mmol) was dissolved in DCM, cyclohexylisothiocyanate (0.004 ml, 0.022 mmol) and NEt₃ (0.003 ml, 0.022 mmol) were added with stirring and left at rt for 7 days, concentrated and purified by chromatography (1-3% methanol in DCM) to afford compound 1109. ¹H-NMR (CD₃OD): δ 8.40 (d, 2H), 7.35 (d, 2H), 4.17 (m, 1H), 4.05 (t, 2H), 3.65 (d, 2H), 3.40 (t, 2H), 1.98 (m, 2H), 1.9-1.55 (m, 12H), 1.5-1.15 (m, 17H), 1.09 (m, 2H).

### Example 110

### 2-cyano-1-(8-(cyclohexylmethoxyamino)hexyl)-3-(pyridin-4-yl)guanidine (Compound 1110)

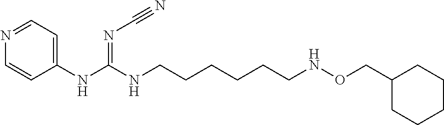

General procedure 5. Starting materials: S-Methyl N-cyano-N′-4-pyridylisothiourea and compound 83. ¹H-NMR (CD₃OD): δ 8.39 (d, 2H), 7.35 (d, 2H), 3.48 (d, 2H), 3.41 (t, 2H), 2.87 (t, 2H), 1.85-1.15 (m, 17H), 0.97 (m, 2H).

### Example 111

### 2-cyano-1-(8-(cyclohexylmethoxyamino)heptyl)-3-(pyridin-4-yl)guanidine (Compound 1111)

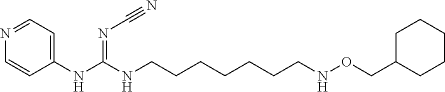

General procedure 5. Starting materials: S-Methyl N-cyano-N′-4-pyridylisothiourea and compound 78. ¹H-NMR (CD₃OD): δ 8.39 (d, 2H), 7.35 (d, 2H), 3.48 (d, 2H), 3.40 (t, 2H), 2.86 (t, 2H), 1.85-1.1 (m, 19H), 0.97 (m, 2H).

### Example 112

### N-(6-(2-cyano-3-(pyridin-4-yl)guanidino)hexyl)-N-(cyclohexylmethoxy)methanesulfonamide (Compound 1112)

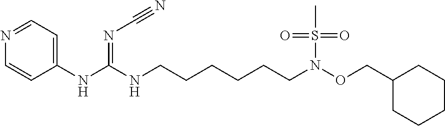

Compound 1110 (18 mg, 0.048 mmol) was dissolved in DCM, methanesulfonyl chloride (0.005 ml, 0.053 mmol) and NEt₃ (0.005 ml, 0.053 mmol) were added with stirring and left at rt overnight, concentrated and purified by chromatography (chloroform:methanol:NH₃ (25% aq.) 98:2:0.2 to 96:4:0.4) to afford compound 1112. ¹H-NMR (CD₃OD): δ 8.39 (d, 2H), 7.35 (d, 2H), 3.86 (d, 2H), 3.42 (t, 2H), 3.21 (t, 2H), 2.93 (s, 3H), 1.85-1.1 (m, 17H), 1.05 (m, 2H).

### Example 113

### N-(6-(2-cyano-3-(pyridin-4-yl)guanidino)hexyl)-N-(cyclohexylmethoxy)-2,2,2-trifluoroethanesulfonamide (Compound 1113)

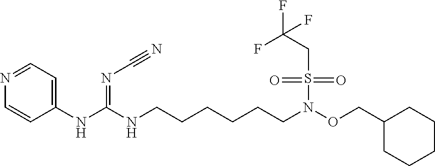

Compound 1110 (21 mg, 0.056 mmol) was dissolved in DCM, 2,2,2-trifluorethanesulfonyl chloride (0.007 ml, 0.067 mmol) and NEt₃ (0.009 ml, 0.067 mmol) were added with stirring and left at rt overnight, concentrated and purified by chromatography (chloroform:methanol:NH₃ (25% aq.) 98:2:0.2 to 97:3:0.3) to afford compound 1113. ¹H-NMR (CD₃OD): δ 8.39 (d, 2H), 7.35 (d, 2H), 4.22 (q, 2H), 3.89 (d, 2H), 3.42 (t, 2H), 3.29 (t, 2H), 1.85-1.1 (m, 17H), 1.06 (m, 2H).

### Example 114

### 1-(6-(2-cyano-3-(pyridin-4-yl)guanidino)hexyl)-1-(cyclohexylmethoxy)-3-ethylurea (Compound 1114)

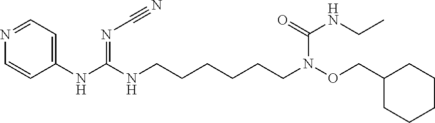

Compound 1110 (20 mg, 0.056 mmol) was dissolved in DCM, ethyl isocyanate (0.005 ml, 0.067 mmol) and NEt₃ (0.009 ml, 0.067 mmol) were added with stirring and left at rt for 3 days, concentrated and purified by chromatography (1-5% methanol in DCM) to afford compound 1114. ¹H-NMR (CD₃OD): δ 8.39 (d, 2H), 7.34 (d, 2H), 6.74 (t, 1H), 3.62 (d, 2H), 3.49 (t, 2H), 3.40 (t, 2H), 3.23 (m, 2H), 1.9-1.5 (m, 9H), 1.5-1.15 (m, 8H), 1.14 (t, 3H), 1.04 (m, 2H).

### Example 115

### 1-(6-(2-cyano-3-(pyridin-4-yl)guanidino)hexyl)-1-(cyclohexylmethoxy)-3-isopropylurea (Compound 1115)

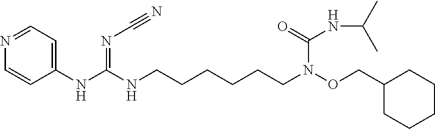

Compound 1110 (20 mg, 0.056 mmol) was dissolved in DCM, isopropyl isocyanate (0.006 ml, 0.067 mmol) and NEt₃ (0.009 ml, 0.067 mmol) were added with stirring and left at rt for 7 days, concentrated and purified by chromatography (1-5% methanol in DCM) to afford compound 1115. ¹H-NMR (CD₃OD): δ 8.39 (d, 2H), 7.34 (d, 2H), 6.28 (d, 1H), 3.86 (m, 1H), 3.63 (d, 2H), 3.49 (t, 2H), 3.40 (t, 2H), 1.9-1.5 (m, 9H), 1.5-1.15 (m, 8H), 1.18 (d, 6H), 1.06 (m, 2H).

### Example 116

### 1-(6-(2-cyano-3-(pyridin-4-yl)guanidino)hexyl)-1-(cyclohexylmethoxy)-3-methylthiourea (Compound 1116)

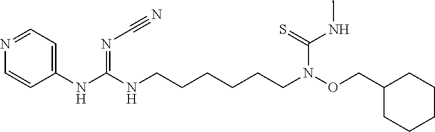

Compound 1110 (24 mg, 0.064 mmol) was dissolved in DCM, methyl isothiocyanate (0.005 ml, 0.077 mmol) and NEt₃ (0.011 ml, 0.077 mmol) were added with stirring and left at rt for 7 days, concentrated and purified by chromatography (1-3% methanol in DCM) to afford compound 1116. ¹H-NMR (CD₃OD): δ 8.39 (d, 2H), 7.35 (d, 2H), 4.07 (t, 2H), 3.64 (d, 2H), 3.41 (t, 2H), 3.05 (s, 3H), 1.9-1.55 (m, 9H), 1.5-1.15 (m, 8H), 1.05 (m, 2H).

### Example 117

### 1-(6-(2-cyano-3-(pyridin-4-yl)guanidino)hexyl)-3-cyclohexyl-1-(cyclohexylmethoxy)thiourea (Compound 1117)

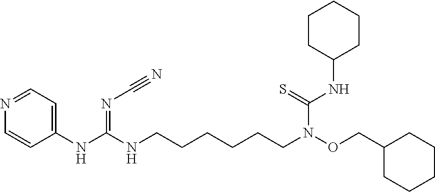

Compound 1110 (20 mg, 0.054 mmol) was dissolved in DCM, cyclohexyl isothiocyanate (0.009 ml, 0.065 mmol) and NEt₃ (0.009 ml, 0.065 mmol) were added with stirring and left at rt for 7 days, concentrated and purified by chromatography (1-3% methanol in DCM) to afford compound 1117. ¹H-NMR (CD₃OD): δ 8.39 (d, 2H), 7.35 (bs, 2H), 4.17 (m, 1H), 4.07 (t, 2H), 3.65 (d, 2H), 3.40 (t, 2H), 1.98 (m, 2H), 1.74 (m, 13H), 1.35 (m, 12H), 1.08 (m, 2H).

### Example 118

### N-(7-(2-cyano-3-(pyridin-4-yl)guanidino)heptyl)-N-(cyclohexylmethoxy)methanesulfonamide (Compound 1118)

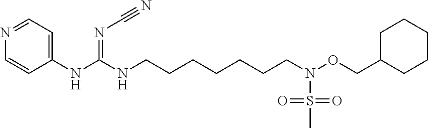

Compound 1111 (20 mg, 0.052 mmol) was dissolved in DCM, methanesulfonyl chloride (0.005 ml, 0.062 mmol) and NEt₃ (0.008 ml, 0.062 mmol) were added with stirring and left at rt overnight, concentrated and purified by chromatography (chloroform:methanol:NH₃ (25% aq.) 98:2:0.2 to 96:4:0.4) to afford compound 1118. ¹H-NMR (CD₃OD): δ 8.39 (d, 2H), 7.34 (d, 2H), 3.86 (d, 2H), 3.41 (t, 2H), 3.20 (t, 2H), 2.93 (s, 3H), 1.85-1.15 (m, 19H), 1.05 (m, 2H).

### Example 119

### N-(7-(2-cyano-3-(pyridin-4-yl)guanidino)heptyl)-N-(cyclohexylmethoxy)-2,2,2-trifluoroethanesulfonamide (Compound 1119)

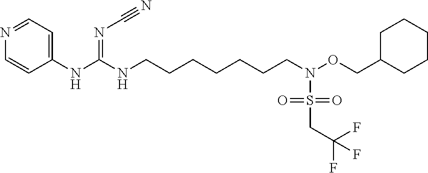

Compound 1111 (22 mg, 0.057 mmol) was dissolved in DCM, 2,2,2-trifluoroethanesulfonyl chloride (0.008 ml, 0.068 mmol) and NEt₃ (0.010 ml, 0.068 mmol) were added with stirring and left at rt overnight, concentrated and purified by chromatography (chloroform:methanol:NH₃ (25% aq.) 98:2:0.2 to 96:4:0.4) to afford compound 1119. ¹H-NMR (CD₃OD): δ 8.39 (d, 2H), 7.34 (d, 2H), 4.23 (q, 2H), 3.90 (d, 2H), 3.42 (t, 2H), 3.28 (t, 2H), 1.85-0.9 (m, 21H).

### Example 120

### 1-(7-(2-cyano-3-(pyridin-4-yl)guanidino)heptyl)-N-(cyclohexylmethoxy)-3-ethylurea (Compound 1120)

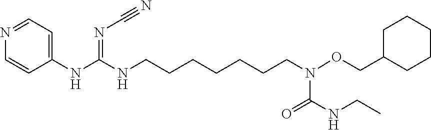

Compound 1111 (27 mg, 0.070 mmol) was dissolved in DCM, ethyl isocyanate (0.0065 ml, 0.084 mmol) and NEt₃ (0.012 ml, 0.084 mmol) were added with stirring and left at rt for seven days, concentrated and purified by chromatography (1-4% methanol in DCM) to afford compound 1120. ¹H-NMR (CD₃OD): δ 8.39 (d, 2H), 7.35 (d, 2H), 6.74 (t, 1H), 3.61 (d, 2H), 3.48 (t, 2H), 3.40 (t, 2H), 3.23 (m, 2H), 1.9-1.5 (m, 10H), 1.45-1.15 (m, 9H), 1.14 (t, 3H), 1.03 (m, 2H).

### Example 121

### 1-(7-(2-cyano-3-(pyridin-4-yl)guanidino)heptyl)-N-(cyclohexylmethoxy)-3-isopropylurea (Compound 1121)

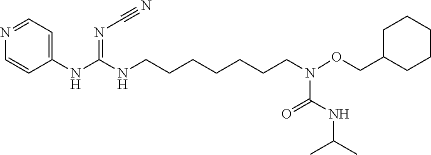

Compound 1111 (24 mg, 0.062 mmol) was dissolved in DCM, isopropyl isocyanate (0.007 ml, 0.074 mmol) and NEt₃ (0.010 ml, 0.074 mmol) were added with stirring and left at rt for seven days, concentrated and purified by chromatography (1-4% methanol in DCM) to afford compound 1121. ¹H-NMR (CD₃OD): δ 8.39 (d, 2H), 7.35 (d, 2H), 6.28 (d, 1H), 3.87 (m, 1H), 3.62 (d, 2H), 3.48 (t, 2H), 3.40 (t, 2H), 1.9-1.5 (m, 10H), 1.5-1.15 (m, 9H), 1.18 (d, 6H), 1.06 (m, 2H).

### Example 122

### 1-(7-(2-cyano-3-(pyridin-4-yl)guanidino)heptyl)-N-(cyclohexylmethoxy)-3-methylthiourea (Compound 1122)

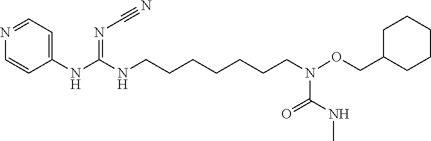

Compound 1111 (21 mg, 0.054 mmol) was dissolved in DCM, methyl isothiocyanate (0.005 ml, 0.065 mmol) and NEt₃ (0.009 ml, 0.065 mmol) were added with stirring and left at rt for seven days, concentrated and purified by chromatography (1-3% methanol in DCM) to afford compound 1122. ¹H-NMR (CD₃OD): δ 8.39 (d, 2H), 7.35 (d, 2H), 4.05 (t, 2H), 3.64 (d, 2H), 3.40 (t, 2H), 3.05 (s, 3H), 1.9-1.5 (m, 10H), 1.5-1.15 (m, 9H), 1.05 (m, 2H).

### Example 123

### 1-(7-(2-cyano-3-(pyridin-4-yl)guanidino)heptyl)-3-cyclohexyl-1-(cyclohexylmethoxy)thiourea (Compound 1123)

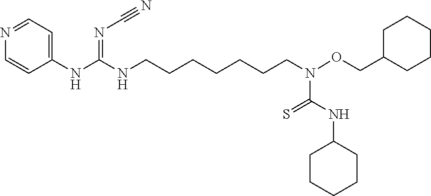

Compound 1111 (24 mg, 0.062 mmol) was dissolved in DCM, cyclohexyl isothiocyanate (0.010 ml, 0.074 mmol) and NEt₃ (0.010 ml, 0.074 mmol) were added with stirring and left at rt for seven days, concentrated and purified by chromatography (1-3% methanol in DCM) to afford compound 1123. ¹H-NMR (CD₃OD): δ 8.39 (d, 2H), 7.35 (d, 2H), 4.16 (m, 1H), 4.06 (t, 2H), 3.65 (d, 2H), 3.40 (t, 2H), 1.99 (m, 2H), 1.9-1.55 (m, 13H), 1.5-1.15 (m, 14H), 1.09 (m, 2H).

### Example 124

### N-(8-(2-cyano-3-(pyridin-4-yl)guanidino)octyl)-N-(cyclohexylmethoxy)methanesulfonamide (Compound 1124)

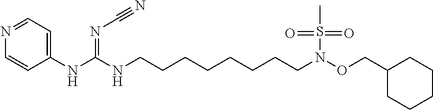

Compound 1107 (11 mg, 0.027 mmol) was dissolved in DCM, methanesulfonyl chloride (0.003 ml, 0.032 mmol) and NEt₃ (0.005 ml, 0.032 mmol) were added with stirring and left at rt overnight, concentrated and purified by chromatography (chloroform:methanol:NH₃ (25% aq.) 98:2:0.2 to 95:5:0.5) to afford compound 1124. ¹H-NMR (CD₃OD): δ 8.39 (d, 2H), 7.35 (d, 2H), 3.86 (d, 2H), 3.41 (t, 2H), 3.19 (t, 2H), 2.92 (s, 3H), 1.85-1.15 (m, 21H), 1.06 (m, 2H).

### Example 125

### 1-(8-(2-cyano-3-(pyridin-4-yl)guanidino)octyl)-N-(cyclohexylmethoxy)-3-ethylurea (Compound 1125)

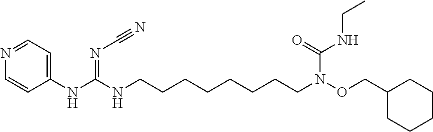

Compound 1107 (18 mg, 0.045 mmol) was dissolved in DCM, ethyl isocyanate (0.0043 ml, 0.054 mmol) and NEt₃ (0.008 ml, 0.054 mmol) were added with stirring and left at rt for seven days, concentrated and purified by chromatography (1-5% methanol in DCM) to afford compound 1125. ¹H-NMR (CD₃OD): δ 8.39 (m, 2H), 7.35 (m, 2H), 6.73 (t, 1H), 3.62 (d, 2H), 3.47 (t, 2H), 3.40 (t, 2H), 3.23 (m, 2H), 1.9-1.5 (m, 10H), 1.45-1.15 (m, 11H), 1.14 (t, 3H), 1.05 (m, 2H).

### Example 126

### 1-(8-(2-cyano-3-(pyridin-4-yl)guanidino)octyl)-N-(cyclohexylmethoxy)-3-isopropylurea (Compound 1126)

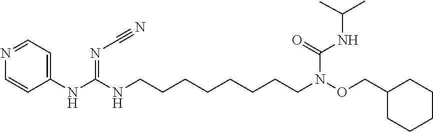

Compound 1107 (18 mg, 0.045 mmol) was dissolved in DCM, isopropyl isocyanate (0.005 ml, 0.054 mmol) and NEt₃ (0.008 ml, 0.054 mmol) were added with stirring and left at rt for seven days, concentrated and purified by chromatography (1-5% methanol in DCM) to afford compound 1126. ¹H-NMR (CD₃OD): δ 8.39 (d, 2H), 7.34 (d, 2H), 6.28 (d, 1H), 3.87 (m, 1H), 3.62 (d, 2H), 3.47 (t, 2H), 3.40 (t, 2H), 1.9-1.5 (m, 10H), 1.5-1.15 (m, 11H), 1.18 (d, 6H), 1.06 (m, 2H).

### Example 127

### 1-(8-(2-cyano-3-(pyridin-4-yl)guanidino)octyl)-N-(cyclohexylmethoxy)-3-methylthiourea (Compound 1127)

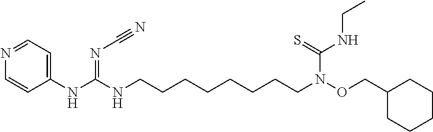

Compound 1107 (25 mg, 0.062 mmol) was dissolved in DCM, methyl isothiocyanate (0.005 ml, 0.074 mmol) and NEt₃ (0.010 ml, 0.074 mmol) were added with stirring and left at rt for seven days, concentrated and purified by chromatography (1-3% methanol in DCM) to afford compound 1127. ¹H-NMR (CD₃OD): δ 8.39 (d, 2H), 7.35 (d, 2H), 4.04 (t, 2H), 3.64 (d, 2H), 3.40 (t, 2H), 3.05 (s, 3H), 1.9-1.1 (m, 21H), 1.05 (m, 2H).

### Example 128

### N-(6-(2-cyano-3-(pyridin-4-yl)guanidino)hexyl)-N-(2-fluoroethyl)cyclohexansulfonamide (Compound 1128)

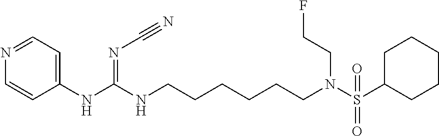

General procedure 5. Starting materials: S-Methyl N-cyano-N′-4-pyridylisothiourea and compound 85. ¹H-NMR (CD₃OD): δ 8.39 (d, 2H), 7.35 (d, 2H), 4.56 (dt, 2H), 3.58 (dt, 2H), 3.41 (t, 2H), 3.33 (t, 2H), 3.11 (m, 1H), 2.10 (m, 2H), 1.88 (m, 2H), 1.75-1.1 (m, 14H).

### Example 129

### N-(6-(3,4-dioxo-2-(pyridin-4-ylamino)cyclobut-1-enylamino)hexyl)-N-(2-fluoroethyl)cyclohexanesulfonamide (Compound 1129)

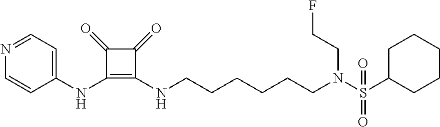

General procedure 7. Starting materials: 3-ethoxy-4-(pyridin-4-ylamino)cyclobut-3-ene-2,3-dione and compound 85. ¹H-NMR (CD₃OD): δ 8.38 (m, 2H), 7.55 (m, 2H), 4.55 (dt, 2H), 3.74 (t, 2H), 3.57 (dt, 2H), 3.32 (t, 2H), 3.09 (m, 1H), 2.07 (m, 2H), 1.86 (m, 2H), 1.68 (m, 5H), 1.55-1.05 (m, 9H).

### Example 130

### (E)-N-(6-(N-(2-fluoroethyl)cyclohexanesulfonamido)hexyl)-3-(pyridin-3-yl)acrylamide (Compound 1130)

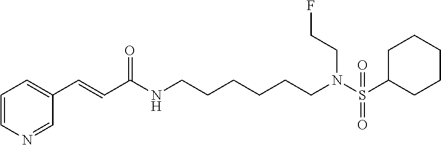

General procedure 10. Starting materials: (E)-3-(pyridin-3-yl)acrylic acid and compound 85. ¹H-NMR (CD₃OD): δ 8.73 (d, 1H), 8.53 (dd, 1H), 8.06 (dt, 1H), 7.56 (d, 1H), 7.49 (m, 1H), 6.75 (d, 1H), 4.55 (dt, 2H), 3.57 (dt, 2H), 3.33 (m, 4H), 3.10 (m, 1H), 2.09 (m, 2H), 1.87 (m, 2H), 1.75-1.1 (m, 14H).

### Example 131

### N-(2-fluoroethyl)-N-(6-(3-pyridin-4-ylthioureido)hexyl)cyclohexanesulfonamide (Compound 1131)

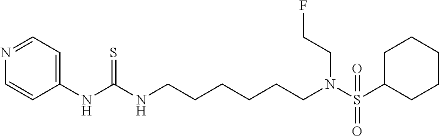

General procedure 12. Starting materials: 4-aminopyridine and compound 85. ¹H-NMR (CD₃OD): δ 8.35 (m, 2H), 7.72 (m, 2H), 4.56 (dt, 2H), 3.62 (m, 3H), 3.53 (t, 1H), 3.31 (t, 2H), 3.10 (m, 1H), 2.09 (m, 2H), 1.88 (m, 2H), 1.67 (m, 5H), 1.6-1.1 (m, 9H).

### Example 132

### 2-cyano-1-(7-morpholinoheptyl)-3-(pyridin-4-yl)guanidine (Compound 1132)

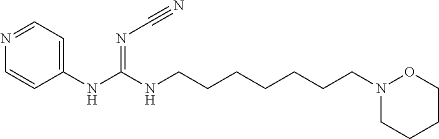

General procedure 5. Starting materials: S-Methyl N-cyano-N′-4-pyridylisothiourea and compound 87. ¹H-NMR (CD₃OD): δ 8.39 (m, 2H), 7.36 (m, 2H), 3.89 (t, 2H), 3.40 (t, 2H), 2.71 (bs, 2H), 2.61 (t, H), 1.79 (m, 2H), 1.59 (m, 6H), 1.40 (m, 6H).

### Example 133

### 3-(7-morpholinoheptylamino)-4-(pyridin-4-ylamino)cyclobut-3-ene-1,2-dione (Compound 1133)

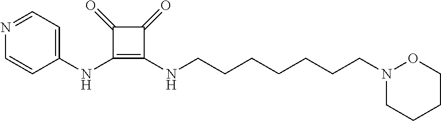

General procedure 7. Starting materials: 3-ethoxy-4-(pyridin-4-ylamino)cyclobut-3-ene-2,3-dione and compound 87. ¹H-NMR (DMSO-d₆): δ 9.89 (bs, 1H), 8.41 (m, 2H), 7.81 (t, 1H), 7.43 (m, 2H), 3.76 (t, 2H), 3.61 (q, 2H), 2.64 (bs, 4H), 1.66 (m, 2H), 1.67 (m, 2H), 1.43 (m, 4H), 1.30 (m, 6H).

### Example 134

### 1-(7-morpholinoheptylamino)-3-(pyridin-3-ylmethyl)urea (Compound 1134)

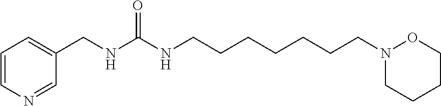

General procedure 6. Starting materials: 3-picolylamine and compound 87. ¹H-NMR (CD₃OD): δ 8.49 (d, 1H), 8.43 (dd, 1H), 7.79 (dt, 1H), 7.41 (m, 1H), 4.37 (s, 2H), 3.89 (t, 2H), 3.14 (t, 2H), 2.71 (bs, 2H), 2.61 (t, 2H), 1.79 (m, 2H), 1.54 (m, 6H), 1.35 (m, 6H).

### Example 135

### (E)-N-(7-morpholinoheptyl)-3-(pyridin-3-yl)acrylamide (Compound 1135)

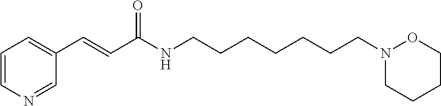

General procedure 10. Starting materials: (E)-3-(pyridin-3-yl)acrylic acid and compound 87. ¹H-NMR (CD₃OD): δ 8.71 (d, 1H), 8.52 (dd, 1H), 8.05 (dt, 1H), 7.56 (d, 1H), 7.48 (m, 1H), 6.75 (d, 1H), 3.88 (t, 2H), 3.32 (t, 2H), 2.740 (bs, 2H), 2.60 (t, 2H), 1.78 (m, 2H), 1.57 (m, 6H), 1.38 (m, 6H).

### Example 136

### 1-(7-morpholinoheptyl)-3-(pyridin-4-yl)thiourea (Compound 1136)

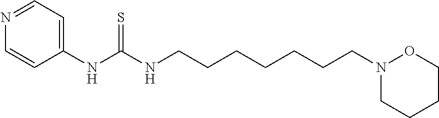

General procedure 12. Starting materials: 4-aminopyridine and compound 87. ¹H-NMR (CD₃OD): δ 8.35 (m, 2H), 7.72 (m, 2H), 3.89 (t, 2H), 3.60 (t, 2H), 2.74 (bs, 2H), 2.62 (t, 2H), 1.79 (m, 2H), 1.66 (m, 2H), 1.57 (m, 4H), 1.40 (m, 6H).

### Example 137

### N-(6-(2-cyano-3-(pyridin-4-yl)guanidino)hexyl)-N-(cyclohexylmethoxy)Propane-2-sulfonamide (Compound 1137)

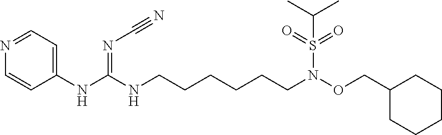

Compound 1110 (18 mg, 0.048 mmol) was dissolved in DCM, isopropylsulfonyl chloride (0.007 ml, 0.058 mmol) and NEt₃ (0.008 ml, 0.058 mmol) were added with stirring and left at rt overnight, concentrated and purified by chromatography (chloroform:methanol:NH₃ (25% aq.) 98:2:0.2 to 96:4:0.4) to afford compound 1137. MS [M+H]⁺=479.4, [M−H]⁻=477.4.

### Example 138

### N-(6-(2-cyano-3-(pyridin-4-yl)guanidino)hexyl)-N-(cyclohexylmethoxy)ethane-2-sulfonamide (Compound 1138)

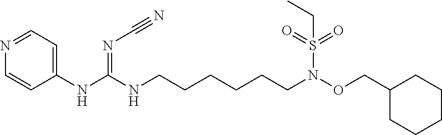

Compound 1110 (12 mg, 0.032 mmol) was dissolved in DCM, ethylsulfonyl chloride (0.004 ml, 0.038 mmol) and NEt₃ (0.005 ml, 0.038 mmol) were added with stirring and left at rt overnight, concentrated and purified by chromatography (chloroform:methanol:NH₃ (25% aq.) 98:2:0.2 to 96:4:0.4) to afford compound 1138. ¹H-NMR (CD₃OD): δ 8.40 (d, 2H), 7.35 (d, 2H), 3.85 (d, 2H), 3.42 (t, 2H), 3.26 (t, 2H), 3.17 (q, 2H), 1.73 (m, 10H), 1.49 (m, 4H), 1.40 (t, 3H), 1.27 (m, 3H), 1.05 (m, 2H).

### Example 139

### N-(6-(2-cyano-3-(pyridin-4-yl)guanidino)hexyl)-N-(cyclohexylmethoxy)cyclopropane-2-sulfonamide (Compound 1139)

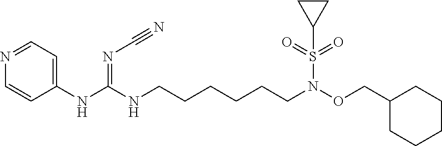

Compound 1110 (14 mg, 0.038 mmol) was dissolved in DCM, cyclopropylsulfonyl chloride (6.3 mg, 0.046 mmol) and NEt₃ (0.006 ml, 0.046 mmol) were added with stirring and left at rt overnight, concentrated and purified by chromatography (chloroform:methanol:NH₃ (25% aq.) 98:2:0.2 to 97:3:0.3) to afford compound 1139. MS [M+H]⁺=477.4, [M−H+HCOOH]⁻=520.8.

### Example 140

### N-(6-(2-cyano-3-(pyridin-4-yl)guanidino)hexyl)-N-(cyclohexylmethoxy)-1,1,1-trifluoromethanesulfonamide (Compound 1140)

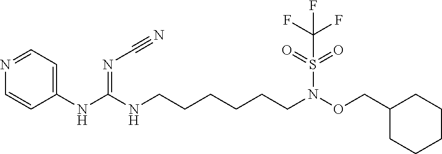

Compound 1110 (15 mg, 0.040 mmol) was dissolved in DCM, trifluoromethanesulfonyl chloride (0.005 ml, 0.048 mmol) and NEt₃ (0.007 ml, 0.048 mmol) were added with stirring and left at rt overnight, concentrated and purified by chromatography (1% to 2% methanol in DCM) to afford compound 1140. MS [M+H]⁺=505.3, [M−H+HCOOH]⁻=503.2.

### Example 141

### 2-cyano-1-(5-(cyclohexylmethoxyamino)pentyl)-3-(pyridin-4-yl)guanidine (Compound 1141)

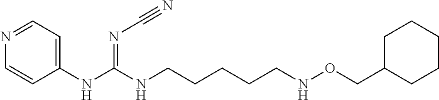

General procedure 5. Starting materials: S-Methyl N-cyano-N′-4-pyridylisothiourea and compound 92. ¹H-NMR (CD₃OD): δ 8.39 (m, 2H), 7.35 (m, 2H), 3.48 (d, 2H), 3.41 (t, 2H), 2.89 (t, 2H), 1.85-1.5 (m, 10H), 1.46 (m, 2H), 1.26 (m, 3H), 0.97 (m, 2H).

### Example 142

### 3-(5-(cyclohexylmethoxyamino)pentylamino)-4-(pyridin-4-ylamino)cyclobut-3-ene-1,2-dione (Compound 1142)

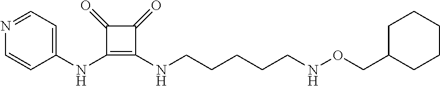

General procedure 7. Starting materials: 3-ethoxy-4-(pyridin-4-ylamino)cyclobut-3-ene-2,3-dione and compound 92. ¹H-NMR (DMSO-d₆): δ 9.89 (bs, 1H), 8.41 (m, 2H), 7.80 (t, 1H), 7.43 (m, 2H), 6.42 (bs, 1H), 3.61 (q, 2H), 3.34 (d, 2H), 2.74 (t, 2H), 1.75-1.0 (m, 14H), 0.86 (m, 3H).

### Example 143

### N-(5-(2-cyano-3-(pyridin-4-yl)guanidino)pentyl)-N-cyclohexylmethoxy)methanesulfonamide (Compound 1143)

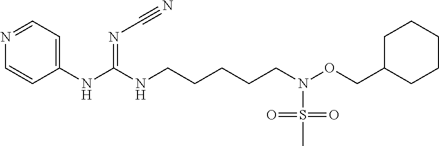

Compound 1141 (18 mg, 0.05 mmol) was dissolved in DCM, methanesulfonyl chloride (0.005 ml, 0.06 mmol) and NEt₃ (0.008 ml, 0.06 mmol) were added with stirring and left at rt overnight, concentrated and purified by chromatography (chloroform:methanol:NH₃ (25% aq.) 98:2:0.2 to 96:4:0.4) to afford compound 1143. ¹H-NMR (CD₃OD): δ 8.40 (m, 2H), 7.35 (m, 2H), 3.87 (d, 2H), 3.43 (t, 2H), 3.33 (t, 2H), 2.93 (s, 3H), 1.85-1.15 (m, 15H), 1.06 (m, 2H).

### Example 144

### N-(5-(2-cyano-3-(pyridin-4-yl)guanidino)pentyl)-N-cyclohexylmethoxy)ethanesulfonamide (Compound 1144)

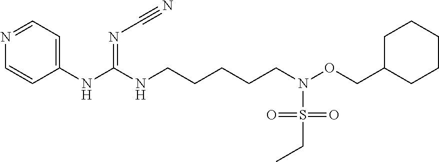

Compound 1141 (18 mg, 0.05 mmol) was dissolved in DCM, ethanesulfonyl chloride (0.006 ml, 0.06 mmol) and NEt₃ (0.008 ml, 0.06 mmol) were added with stirring and left at rt overnight, concentrated and purified by chromatography (chloroform:methanol:NH₃ (25% aq.) 98:2:0.2 to 96:4:0.4) to afford compound 1144. ¹H-NMR (CD₃OD): δ 8.40 (m, 2H), 7.35 (m, 2H), 3.86 (d, 2H), 3.43 (t, 2H), 3.28 (t, 2H), 3.18 (q, 2H), 1.85-1.45 (m, 12H), 1.40 (t, 3H), 1.27 (m, 3H), 1.05 (m, 2H).

### Example 145

### 1-(5-(2-cyano-3-(pyridin-4-yl)guanidino)pentyl)-1-(cyclohexylmethoxy)-3-isopropylurea (Compound 1145)

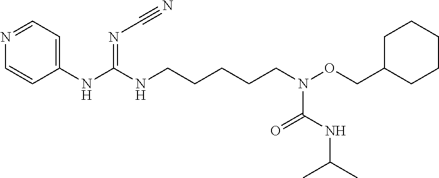

Compound 1141 (20 mg, 0.06 mmol) was dissolved in DCM, isopropyl isocyanate (0.007 ml, 0.07 mmol) and NEt₃ (0.009 ml, 0.07 mmol) were added with stirring and left at rt overnight, concentrated and purified by chromatography (chloroform:methanol:NH₃ (25% aq.) 98:2:0.2 to 97:3:0.3) to afford compound 1145. ¹H-NMR (CD₃OD): δ 8.39 (m, 2H), 7.35 (m, 2H), 6.27 (d, 1H), 3.88 (m, 1H), 3.63 (d, 2H), 3.51 (t, 2H), 3.40 (t, 2H), 1.72 (m, 10H), 1.35 (m, 5H), 1.18 (d, 6H), 1.06 (m, 2H).

### Example 146

### 1-(5-(2-cyano-3-(pyridin-4-yl)guanidino)pentyl)-1-(cyclohexylmethoxy)-3-ethylurea (Compound 1146)

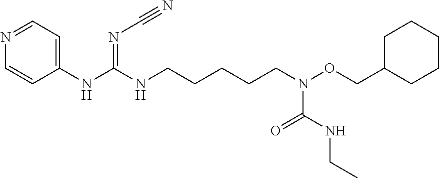

Compound 1141 (20 mg, 0.06 mmol) was dissolved in DCM, ethyl isocyanate (0.005 ml, 0.07 mmol) and NEt₃ (0.009 ml, 0.07 mmol) were added with stirring and left at rt overnight, concentrated and purified by chromatography (chloroform:methanol:NH₃ (25% aq.) 98:2:0.2 to 97:3:0.3) to afford compound 1146. ¹H-NMR (CD₃OD): δ 8.39 (m, 2H), 7.35 (m, 2H), 6.72 (t, 1H), 3.63 (d, 2H), 3.51 (t, 2H), 3.40 (t, 2H), 3.23 (m, 2H), 1.73 (m, 10H), 1.34 (m, 5H), 1.13 (t, 3H), 1.06 (m, 2H).

### Example 147

### 1-(5-(2-cyano-3-(pyridin-4-yl)guanidino)pentyl)-1-(cyclohexylmethoxy)-3-methylthiourea (Compound 1147)

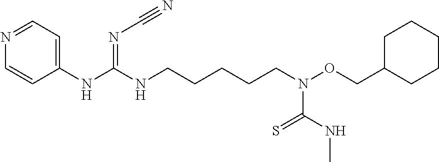

Compound 1141 (20 mg, 0.06 mmol) was dissolved in DCM, methyl isothiocyanate (0.005 ml, 0.07 mmol) and NEt₃ (0.009 ml, 0.07 mmol) were added with stirring and left at rt overnight, concentrated and purified by chromatography (2-4% methanol in DCM) to afford compound 1147. ¹H-NMR (CD₃OD): δ 8.40 (m, 2H), 7.35 (m, 2H), 4.10 (t, 2H), 3.65 (d, 2H), 3.41 (t, 2H), 3.05 (s, 3H), 1.75 (m, 10H), 1.38 (m, 5H), 1.06 (m, 2H).

### Example 148

### N-(5-(2-cyano-3-(pyridin-4-yl)guanidino)pentyl)-N-(cyclohexylmethoxy)benzenesulfonamide (Compound 1148)

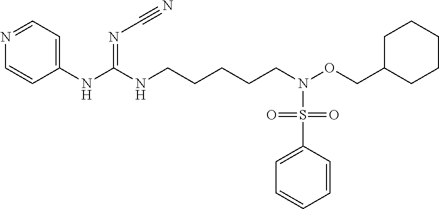

Compound 1141 (27 mg, 0.08 mmol) was dissolved in DCM, benzenesulfonyl chloride (0.012 ml, 0.09 mmol) and NEt₃ (0.013 ml, 0.09 mmol) were added with stirring and left at rt overnight, concentrated and purified by chromatography (chloroform:methanol:NH₃ (25% aq.) 98:2:0.2 to 97:3:0.3) to afford compound 1148. ¹H-NMR (CD₃OD): δ 8.40 (m, 2H), 7.88 (m, 2H), 7.75 (m, 1H), 7.64 (m, 2H), 7.35 (m, 2H), 3.92 (d, 2H), 3.41 (t, 2H), 2.93 (bs 2H), 1.69 (m, 10H), 1.52 (m, 2H), 1.27 (m, 3H), 1.05 (m, 2H).

### Example 149

### N-(5-(2-cyano-3-(pyridin-4-yl)guanidino)pentyl)-N-(cyclohexylmethoxy)Propane-2-sulfonamide (Compound 1149)

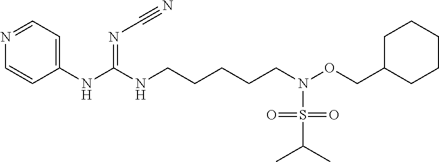

Compound 1141 (28 mg, 0.08 mmol) was dissolved in DCM, isopropylsulfonyl chloride (0.011 ml, 0.09 mmol) and NEt₃ (0.013 ml, 0.09 mmol) were added with stirring and left at rt overnight, concentrated and purified by chromatography (2-4% methanol in DCM) to afford compound 1149. ¹H-NMR (CD₃OD): δ 8.42 (m, 2H), 7.41 (m, 2H), 3.85 (d, 2H), 3.58 (m, 1H), 3.45 (t, 2H), 3.37 (t, 2H), 1.74 (m, 10H), 1.56 (m, 2H), 1.41 (d, 6H), 1.30 (m, 3H), 1.06 (m, 2H).

### Example 150

### N-(benzyloxy)-N-(8-(3-(pyridin-4-yl)ureido)octyl)methanesulfonamide (Compound 1150)

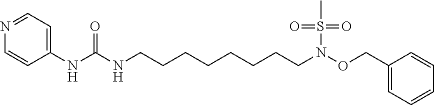

General procedure 11. Starting materials: 4-aminopyridine and compound 24. ¹H-NMR (CD₃OD): δ 8.26 (m, 2H), 7.45 (m, 2H), 7.38 (m, 5H), 5.00 (s, 2H), 3.21 (t, 2H), 3.16 (t, 2H), 2.94 (s, 3H), 1.55 (m, 4H), 1.33 (m, 8H).

### Example 151

### N-(benzyloxy)-N-(8-(3-(pyridin-4-yl)thioureido)octyl)methanesulfonamide (Compound 1151)

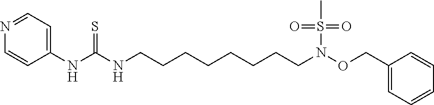

General procedure 12. Starting materials: 4-aminopyridine and compound 24. ¹H-NMR (CD₃OD): δ 8.35 (m, 2H), 7.72 (m, 2H), 7.40 (m, 5H), 5.02 (s, 2H), 3.61 (t, 2H), 3.18 (t, 2H), 2.96 (s, 3H), 1.62 (m, 4H), 1.37 (m, 8H).

### Example 152

### N-(benzyloxy)-N-(6-(2-cyano-3-(pyridin-4-yl)guanidino)hexyl)methanesulfonamide (Compound 1152)

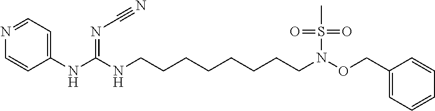

General procedure 5. Starting materials: S-Methyl N-cyano-N′-4-pyridylisothiourea and compound 94. ¹H-NMR (CD₃OD): δ 8.38 (m, 2H), 7.38 (m, 7H), 5.01 (s, 2H), 3.39 (t, 2H), 3.18 (t, 2H), 2.95 (s, 3H), 1.60 (m, 4H), 1.38 (m, 4H).

### Example 153

### N-(benzyloxy)-N-(6-(3,4-dioxo-2-(pyridin-4-ylamino)cyclobut-1-enylamino)hexyl)methanesulfonamide (Compound 1153)

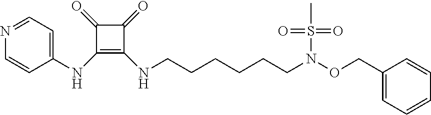

General procedure 7. Starting materials: 3-ethoxy-4-(pyridin-4-ylamino)cyclobut-3-ene-2,3-dione and compound 94. ¹H-NMR (DMSO-d₆): δ 9.89 (bs, 1H), 8.41 (d, 2H), 7.79 (t, 1H), 7.43 (d, 2H), 7.38 (m, 5H), 4.95 (s, 2H), 3.61 (m, 2H), 3.12 (t, 2H), 3.03 (s, 3H), 1.55 (m, 4H), 1.33 (m, 4H).

### Example 154

### N-(benzyloxy)-N-(6-(3-(pyridin-3-ylmethyl)ureido)hexyl)methanesulfonamide (Compound 1154)

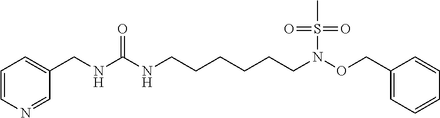

General procedure 6. Starting materials: 3-picolylamine and compound 94. ¹H-NMR (CD₃OD): δ 8.49 (d, 1H), 8.41 (dd, 1H), 7.79 (dt, 1H), 7.39 (m, 6H), 5.01 (s, 2H), 4.37 (s, 2H), 3.16 (m, 4H), 2.95 (s, 3H), 1.58 (m, 2H), 1.48 (m, 2H), 1.34 (m, 4H).

### Example 155

### N-(benzyloxy)-N-(6-(3-(pyridin-4-yl)thioureido)hexyl)methanesulfonamide (Compound 1155)

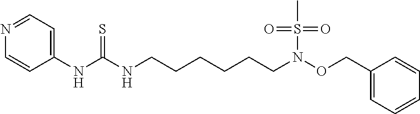

General procedure 12. Starting materials: 4-aminopyridine and compound 94. ¹H-NMR (CD₃OD): δ 8.34 (m, 2H), 7.71 (m, 2H), 7.39 (m, 5H), 5.02 (s, 2H), 3.60 (t, 2H), 3.19 (t, 2H), 2.96 (s, 3H), 1.63 (m, 4H), 1.40 (m, 4H).

### Example 156

### (E)-N-(benzyloxy)methylsulfonamido)hexyl)-3-(pyridin-3-yl)acrylamide (Compound 1156)

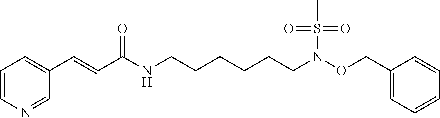

General procedure 10. Starting materials: (E)-3-(pyridin-3-yl)acrylic acid and compound 94. ¹H-NMR (CD₃OD): δ 8.71 (d, 1H), 8.52 (dd, 1H), 8.04 (dt, 1H), 7.55 (d, 1H), 7.47 (m, 1H), 7.38 (m, 5H), 6.75 (d, 1H), 5.01 (s, 2H), 3.33 (t, 2H), 3.18 (t, 2H), 2.95 (s, 3H), 1.59 (m, 4H), 1.38 (m, 4H).

### Example 157

### N-(benzyloxy)-N-(6-(3-(pyridin-4-yl)ureido)hexyl)methanesulfonamide (Compound 1157)

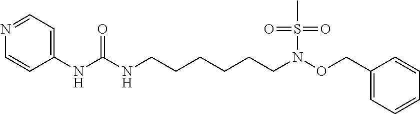

General procedure 11. Starting materials: 4-aminopyridine and compound 94. ¹H-NMR (CD₃OD): δ 8.27 (m, 2H), 7.45 (m, 2H), 7.38 (m, 5H), 5.01 (s, 2H), 3.20 (m, 4H), 2.95 (s, 3H), 1.57 (m, 4H), 1.38 (m, 4H).

### Example 158

### N-(benzyloxy)-N-(6-(2-cyano-3-(pyridin-4-yl)guanidino)heptyl)methanesulfonamide (Compound 1158)

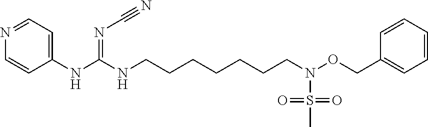

General procedure 5. Starting materials: S-Methyl N-cyano-N′-4-pyridylisothiourea and compound 96. ¹H-NMR (CD₃OD): δ 8.38 (m, 2H), 7.38 (m, 7H), 5.01 (s, 2H), 3.40 (t, 2H), 3.18 (t, 2H), 2.95 (s, 3H), 1.61 (m, 4H), 1.37 (m, 6H).

### Example 153

### N-(benzyloxy)-N-(6-(3,4-dioxo-2-(pyridin-4-ylamino)cyclobut-1-enylamino)heptyl)methanesulfonamide (Compound 1153)

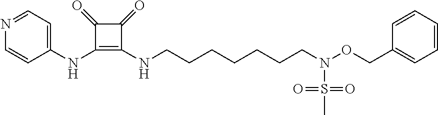

General procedure 7. Starting materials: 3-ethoxy-4-(pyridin-4-ylamino)cyclobut-3-ene-2,3-dione and compound 96. NMR (DMSO-d₆): δ 9.88 (bs, 1H), 8.41 (d, 2H), 7.80 (t, 1H), 7.43 (d, 2H), 7.39 (m, 5H), 4.95 (s, 2H), 3.62 (m, 2H), 3.11 (t, 2H), 3.02 (s, 3H), 1.54 (m, 4H), 1.30 (m, 6H).

### Example 154

### N-(benzyloxy)-N-(6-(3-(pyridin-3-ylmethyl)ureido)heptyl)methanesulfonamide (Compound 1154)

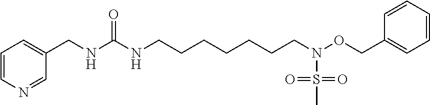

General procedure 6. Starting materials: 3-picolylamine and compound 96. ¹H-NMR (CD₃OD): δ 8.49 (d, 1H), 8.41 (dd, 1H), 7.78 (dt, 1H), 7.39 (m, 6H), 5.01 (s, 2H), 4.36 (s, 2H), 3.16 (m, 4H), 2.95 (s, 3H), 1.57 (m, 2H), 1.49 (m, 2H), 1.32 (m, 6H).

### Example 155

### N-(benzyloxy)-N-(6-(3-(pyridin-4-yl)thioureido)heptyl)methanesulfonamide (Compound 1155)

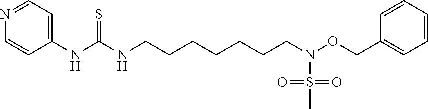

General procedure 12. Starting materials: 4-aminopyridine and compound 96. ¹H-NMR (CD₃OD): δ 8.34 (m, 2H), 7.71 (m, 2H), 7.39 (m, 5H), 5.01 (s, 2H), 3.61 (t, 2H), 3.18 (t, 2H), 2.95 (s, 3H), 1.62 (m, 4H), 1.37 (m, 6H).

### Example 156

### (E)-N-(benzyloxy)methylsulfonamido)heptyl)-3-(pyridin-3-yl)acrylamide (Compound 1156)

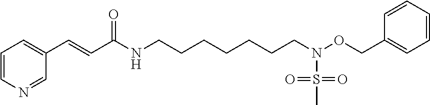

General procedure 10. Starting materials: (E)-3-(pyridin-3-yl)acrylic acid and compound 96. ¹H-NMR (CDCl₃): δ 8.73 (d, 1H), 8.55 (dd, 1H), 7.78 (dt, 1H), 7.61 (d, 1H), 7.37 (m, 5H), 7.28 (m, 1H), 6.51 (d, 1H), 6.04 (t, 1H), 5.01 (s, 2H), 3.38 (q, 2H), 3.15 (t, 2H), 2.88 (s, 3H), 1.58 (m, 4H), 1.32 (m, 6H).

### Example 157

### N-(benzyloxy)-N-(6-(3-(pyridin-4-yl)ureido)heptyl)methanesulfonamide (Compound 1157)

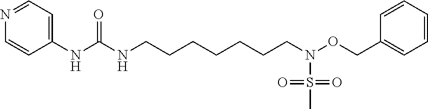

General procedure 11. Starting materials: 4-aminopyridine and compound 96. ¹H-NMR (CD₃OD): δ 8.26 (m, 2H), 7.45 (m, 2H), 7.38 (m, 5H), 5.00 (s, 2H), 3.19 (m, 4H), 2.94 (s, 3H), 1.56 (m, 4H), 1.34 (m, 6H).

### Example 158

### N-(8-(2-cyano-3-pyridin-4-yl)guanidino)octyl)-N-(4-fluorobenzyloxy)methanesulfonamide (Compound 1158)

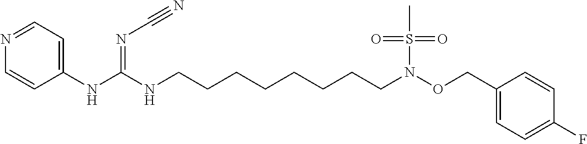

General procedure 5. Starting materials: S-Methyl N-cyano-N′-4-pyridylisothiourea and compound 99. ¹H-NMR (CD₃OD): δ 8.39 (m, 2H), 7.39 (m, 7H), 7.44 (m, 2H), 7.35 (m, 2H), 7.11 (m, 2H), 4.99 (s, 2H), 3.40 (t, 2H), 3.17 (t, 2H), 2.95 (s, 3H), 1.65 (m, 2H), 1.55 (m, 2H), 1.36 (m, 8H).

### Example 159

### N-(8-(3,4-dioxo-2-(pyridin-4-ylamino)cyclobute-1-enylamino)octyl)-N-(4-fluorobenzyloxy)methansulfonamide (Compound 1159)

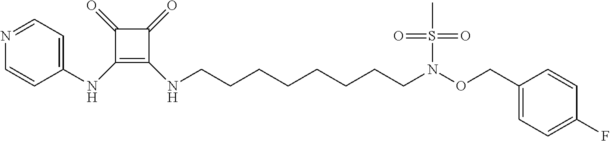

General procedure 7. Starting materials: 3-ethoxy-4-(pyridin-4-ylamino)cyclobut-3-ene-2,3-dione and compound 99. ¹H-NMR (DMSO-d₆): 9.87 (bs, 1H), 8.40 (d, 2H), 7.80 (t, 1H), 7.43 (m, 4H), 7.21 (m, 2H), 4.92 (s, 2H), 3.61 (m, 2H), 3.09 (t, 2H), 3.01 (s, 3H), 1.53 (m, 4H), 1.26 (m, 8H).

### Example 160

### N-(4-fluorobenzyloxy)-N-(8-(3-pyridin-3-ylmethyl)ureido)octyl)methansulfonamide (Compound 1160)

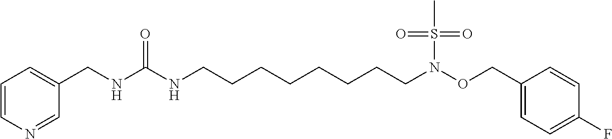

General procedure 6. Starting materials: 3-picolylamine and compound 99. ¹H-NMR (CD₃OD): δ 8.49 (d, 1H), 8.42 (dd, 1H), 7.79 (dt, 1H), 7.41 (m, 3H), 7.11 (m, 2H), 5.00 (s, 2H), 4.37 (s, 2H), 3.15 (m, 4H), 2.95 (s, 3H), 1.52 (m, 4H), 1.32 (m, 8H).

### Example 161

### N-(8-(N-(4-fluorobenzyloxy)methylsulfonamido)octyl-3-(pyridin-3-yl)acrylamide (Compound 1161)

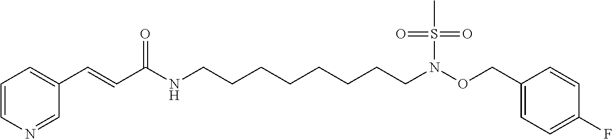

General procedure 10. Starting materials: (E)-3-(pyridin-3-yl)acrylic acid and compound 99. ¹H-NMR (CDCl₃): δ 8.73 (d, 1H), 8.55 (dd, 1H), 7.78 (dt, 1H), 7.60 (d, 1H), 7.36 (m, 2H), 7.29 (m, 1H), 7.04 (m, 2H), 6.50 (d, 1H), 5.98 (t, 1H), 4.97 (s, 2H), 3.38 (q, 2H), 3.13 (bs, 2H), 2.88 (s, 3H), 1.56 (m, 4H), 1.30 (m, 8H).

### Example 162

### N-(4-fluorobenzyloxy)-N-(8-(3-(pyridin-4-yl)ureido)octyl)methanesulfonamide (Compound 1162)

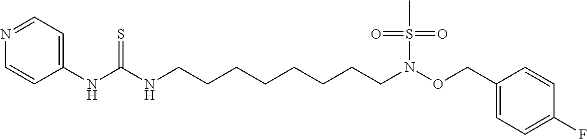

General procedure 11. Starting materials: 4-aminopyridine and compound 99. ¹H-NMR (CD₃OD): δ 8.34 (m, 2H), 7.72 (m, 2H), 7.44 (m, 2H), 7.11 (m, 2H), 5.00 (s, 2H), 3.60 (t, 2H), 3.17 (t, 2H), 2.95 (s, 3H), 1.67 (m, 2H), 1.57 (m, 2H), 1.37 (m, 8H).

### Example 163

### N-(4-fluorobenzyloxy)-N-(8-(3-(pyridin-4-yl)thioureido)octyl)methanesulfonamide (Compound 1163)

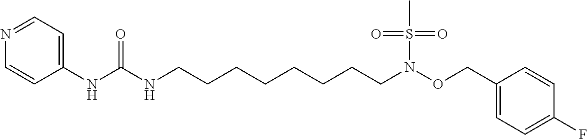

General procedure 12. Starting materials: 4-aminopyridine and compound 99. ¹H-NMR (CD₃OD): δ 8.27 (m, 2H), 7.43 (m, 4H), 7.11 (m, 2H), 4.99 (s, 2H), 3.22 (t, 2H), 3.16 (t, 2H), 2.95 (s, 3H), 1.55 (m, 4H), 1.35 (m, 8H).

### Example 164

### In Vitro Cell Proliferation Assay (WST-1 Assay)

A2780 cells were seeded in 96-well plates at 3×10³ cells/well in 100 μL of culture medium, 8 wells were left empty for media only controls.

After 24 h the compound titrations were performed, in a separate dilution plate, by serially diluting the compounds of general formula (I) in culture medium. A 100 μL of each dilution was added to the plated cells, this was done in triplicate, and controls (e.g. DMSO and blanks) were included. The plates were incubated for 24 h at 37° C. in a CO₂ incubator. The compound titrations were repeated in a separate dilution plate after 24 h. The media plus compound from the assay plates were then aspirated. A 100 μL of media was then added to all wells, followed by 100 μL of each compound dilution. The plates were incubated for a further 48 h at 37° C. in a CO₂ incubator (total incubation time 72 h). The number of viable cells was then assessed using Cell Proliferation Reagent WST-1. 10 μL of WST-1 reagent added to each well and incubated for one to four hours at 37° C. in CO₂ incubator. The absorbance was measured (450 nm/690 nm).

The activity of compounds of general formula (I) in reducing the number of viable cells was calculated as:

% activity=[(S^{c}−B)/(S^{o}−B)]×100

S^{c} denotes signal measured in the presence of test compound, S^{o} denotes signal detected in the absence of compound, and B denotes background signal, measured in blank wells containing medium only. Analyse data using GraphPad Prism.

Results can be seen in Table 1.

TABLE 1
In vitro cell proliferation assay (WST-1
assay as described in Example 164)

| | IC₅₀ |
|---|---|
| | (nM) for |
| Compound No. | A2780 |
| Reference compound CHS828: 1-(6-(4-chloro- | 0.52 |
| phenoxy)hexyl)-2-cyano-3-(pyridine- | |
| 4-yl)guanidine (U.S. Pat. No. 5,696,140) | |
| Compound 1001 | 0.49 |
| Compound 1002 | 2.03 |
| Compound 1003 | 11.2 |
| Compound 1010 | 0.002 |
| Compound 1011 | 0.003 |
| Compound 1012 | 0.09 |
| Compound 1013 | 0.025 |
| Compound 1015 | 0.10 |
| Compound 1016 | 0.44 |
| Compound 1019 | 0.44 |
| Compound 1020 | 0.018 |
| Compound 1022 | 0.80 |
| Compound 1024 | 0.047 |
| Compound 1028 | 0.37 |
| Compound 1033 | 1.17 |
| Compound 1035 | 0.042 |
| Compound 1037 | 0.002 |
| Compound 1046 | 0.38 |
| Compound 1055 | 0.025 |
| Compound 1056 | 0.35 |
| Compound 1057 | 0.37 |
| Compound 1058 | 0.39 |
| Compound 1060 | 0.049 |
| Compound 1061 | 0.35 |
| Compound 1064 | 0.44 |
| Compound 1071 | 0.049 |
| Compound 1072 | 0.17 |
| Compound 1073 | 0.31 |
| Compound 1074 | 0.60 |
| Compound 1075 | 0.39 |
| Compound 1076 | 0.075 |
| Compound 1081 | 0.55 |
| Compound 1085 | 0.062 |
| Compound 1086 | 0.024 |
| Compound 1088 | 5.65 |
| Compound 1093 | 0.063 |
| Compound 1094 | 0.036 |
| Compound 1098 | 0.070 |
| Compound 1099 | 0.43 |
| Compound 1103 | 0.005 |
| Compound 1107 | 0.57 |
| Compound 1110 | 0.015 |
| Compound 1112 | 0.086 |
| Compound 1114 | 0.057 |
| Compound 1115 | 0.033 |
| Compound 1116 | 0.056 |
| Compound 1118 | 0.046 |
| Compound 1124 | 0.15 |
| Compound 1125 | 0.057 |
| Compound 1132 | 0.93 |
| Compound 1138 | 0.059 |
| Compound 1143 | 0.081 |
| Compound 1144 | 0.037 |

1. A compound of the formula (I) 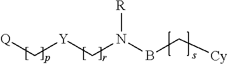
wherein
Q is selected from optionally substituted pyrid-3-yl and optionally substituted pyrid-4-yl;
p is an integer of 0-6;
Y is selected from (i)-(iii): 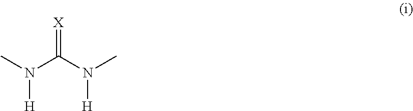
where X is selected from ═O, ═S and ═N—CN, 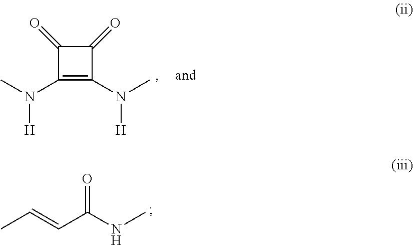
r is an integer of 1-12,
R designates —Z-A, wherein Z is selected from a single bond, —S(═O)₂—, >P═O, >C═O, —C(═O)NH—, and —C(═S)NH—; and A is selected from hydrogen, optionally substituted C₁₋₁₂-alkyl, optionally substituted C₃₋₁₂-cycloalkyl, —[CH₂CH₂O]₁₋₁₀-(optionally substituted C₁₋₆-alkyl), optionally substituted C₁₋₁₂-alkenyl, optionally substituted aryl, optionally substituted heterocyclyl, and optionally substituted heteroaryl;
B is selected from a single bond, —NR^{N}—, —S(═O)₂— and —O—; wherein R^{N} is selected from hydrogen, optionally substituted C₁₋₁₂-alkyl, optionally substituted C₃₋₁₂-cycloalkyl, —[CH₂CH₂O]₁₋₁₀-(optionally substituted C₁₋₆-alkyl), optionally substituted C₁₋₁₂-alkenyl, optionally substituted aryl, optionally substituted heterocyclyl, and optionally substituted heteroaryl;
s is an integer of 0-6; and
Cy is selected from optionally substituted aryl, optionally substituted cycloalkyl, optionally substituted heterocyclyl, and optionally substituted heteroaryl.

2. The compound according to claim 1, wherein Q is selected from pyrid-3-yl and pyrid-4-yl.

3. The compound according to any one of the preceding claims, wherein p is an integer of 0-2.

4. The compound according to claim 3, wherein p is an integer of 0 when Y is a group of the type (ii) or (iii), and p is an integer of 0-1 when Y is a group of the type (i).

5. The compound according to any one of the preceding claims, wherein r is an integer of 5-9.

6. The compound according to any one of the preceding claims, wherein Z is a single bond, and A is optionally substituted C₃₋₈-cycloalkyl.

7. The compound according to any one of the preceding claims, wherein Z is sulfonyl, and A is selected from optionally substituted C₃₋₈-cycloalkyl and optionally substituted C₁₋₆-alkyl.

8. The compound according to any one of the preceding claims, wherein Z is sulfonyl, and A is optionally substituted aryl.

9. The compound according to any one of the preceding claims, wherein r is an integer of 7-10 when Z is a single bond, and r is 6-9 when Z is —S(═O)₂—.

10. The compound according to any one of the preceding claims, wherein when B is a single bond, s is 1-5, and when B is —O—, s is 0-2.

11. The compound according to any one of the preceding claims, wherein Cy is selected from optionally substituted heterocyclyl and optionally substituted aryl.

12. The compound according to any one of the preceding claims, which is selected from the following:
2-cyano-1-(7-(cyclohexyl(3-morpholinopropyl)amino)octyl)-3-(pyridin-4-yl)guanidine,
2-cyano-1-(7-(cyclohexyl(3-morpholinopropyl)amino)heptyl)-3-(pyridin-4-yl)guanidine,
2-cyano-1-(6-(cyclohexyl(3-morpholinopropyl)amino)hexyl)-3-(pyridin-4-yl)guanidine,
1-(8-(cyclohexyl(3-morpholinopropyl)amino)octyl)-3-(pyridin-3-ylmethyl)urea,
1-(7-(cyclohexyl(3-morpholinopropyl)amino)heptyl)-3-(pyridin-3-ylmethyl)urea,
3-(8-(cyclohexyl(3-morpholinopropyl)amino)-4-(pyridin-4-ylamino)cyclobut-3-ene-1,2-dione,
3-(7-cyclohexyl(3-morpholinopropyl)amino)heptylamino)-4-(pyridin-4-ylamino)cyclobut-3-ene-1,2-dione,
N-(8-(2-cyano-3-(pyridin-4-yl)guanidino)octyl)-N-(3-morpholinopropyl)cyclopentanesulfonamide,
N-(8-(2-cyano-3-(pyridin-4-yl)guanidino)octyl)-N-(3-morpholinopropyl)cyclohexanesulfonamide,
N-(7-(2-cyano-3-(pyridin-4-yl)guanidino)heptyl)-N-(3-morpholino propyl)cyclohexanesulfonamide,
N-(7-(2-cyano-3-(pyridin-4-yl)guanidino)heptyl)-N-(3-morpholino propyl)cyclopentanesulfonamide,
N-(3-morpholinopropyl)-N-(8-(3-(pyridin-3-ylmethyl)ureido)octyl)cyclopentanesulfonamide,
N-(3-morpholinopropyl)-N-(8-(3-(pyridin-3-ylmethyl)ureido)octyl)cyclohexanesulfonamide,
N-(3-morpholinopropyl)-N-(7-(3-(pyridin-3-ylmethyl)ureido)heptyl)cyclohexanesulfonamide,
N-(3-morpholinopropyl)-N-(7-(3-(pyridin-3-ylmethyl)ureido)heptyl)cyclopentanesulfonamide,
N-(8-(3,4-dioxo-2-(pyridin-4-ylamino)cyclobut-1-enylamino)octyl)-N-(3-morpholinopropyl)cyclopentanesulfonamide,
N-(8-(3,4-dioxo-2-(pyridin-4-ylamino)cyclobut-1-enylamino)octyl)-N-(3-morpholinopropyl)cyclohexanesulfonamide,
N-(7-(3,4-dioxo-2-(pyridin-4-ylamino)cyclobut-1-enylamino)heptyl)-N-(3-morpholinopropyl)cyclohexanesulfonamide,
N-(7-(3,4-dioxo-2-(pyridin-4-ylamino)cyclobut-1-enylamino)heptyl)-N-(3-morpholinopropyl)cyclopentanesulfonamide,
N-(benzyloxy)-N-(8-(2-cyano-3-pyridin-4-yl)guanidino)octyl)methanesulfonamide,
N-(Benzyloxy)-N-(8-(3,4-dioxo-2-(pyridin-4-ylamino)cyclobute-1-enylamino)octyl)methansulfonamide,
N-(Benzyloxy)-N-(8-(3-pyridin-3-ylmethyl)ureido)octyl)methansulfonamide,
N-(8-(N-Benzyloxy)methylsulfonamido)octyl-3-(pyridin-3-yl)acrylamide,
N-(benzyloxy)-N-(8-(2-cyano-3-pyridin-4-yl)guanidino)octyl)propane-2-sulfonamide,
N-(Benzyloxy)-N-(8-(3,4-dioxo-2-(pyridin-4-ylamino)cyclobute-1-enylamino)octyl)propane-2-sulfonamide,
N-(Benzyloxy)-N-(8-(3-pyridin-3-ylmethyl)ureido)octyl)propane-2-sulfonamide,
N-(8-(N-Benzyloxy)propan-2-ylsulfonamido)octyl)-3-(pyridin-3-yl)acrylamide,
N-(Benzyloxy)-N-(8-(3-pyridin-4-ylureido)octyl)propane-2-sulfonamide,
N-(Benzyloxy)-N-(8-(3-pyridin-4-ylthioureido)octyl)propane-2-sulfonamide,
N-(8-(2-cyano-3-(pyridin-4-yl)guanidino)octyl)-N-(3-morpholinopropyl)methanesulfonamide,
N-(8-(3,4-dioxo-2-(pyridin-4-ylamino)cyclobute-1-enylamino)octyl)-N-(3-morpholinopropyl)methanesulfonamide,
N-(3-Morpholinopropyl)-N-(8-(3-pyridin-3-ylmethyl)ureido)octyl)methanesulfonamide,
N-(8-(N-(3-morpholinopropyl)methylsulfonamido)octyl)-3-(pyridin-3-yl)acrylamide,
N-(3-Morpholinopropyl)-N-(8-(3-pyridin-4-ylthioureido)octyl)methanesulfonamide,
N-(8-(2-cyano-3-(pyridin-4-yl)guanidino)octyl)-N-(3-morpholinopropyl)benzenesulfonamide,
N-(8-(3,4-dioxo-2-(pyridin-4-ylamino)cyclobute-1-enylamino)octyl)-N-(3-morpholinopropyl)benzenesulfonamide,
N-(3-Morpholinopropyl)-N-(8-(3-pyridin-3-ylmethyl)ureido)octyl)benzenesulfonamide,
N-(8-(N-(3-morpholinopropyl)phenylsulfonamido)octyl)-3-(pyridin-3-yl)acrylamide,
N-(3-Morpholinopropyl)-N-(8-(3-pyridin-4-ylureido)octyl)benzenesulfonamide,
N-(3-Morpholinopropyl)-N-(8-(3-pyridin-4-ylureido)octyl)benzenesulfonamide,
1-(7-Cyclohexyl(3-morpholinopropyl)amino)heptyl)-3-(pyridin-4-yl)thiourea oxalate,
1-(7-Cyclohexyl(3-morpholinopropyl)amino)heptyl)-3-(pyridin-4-yl)urea oxalate,
(E)-N-(7-Cyclohexyl(3-morpholinopropyl)amino)heptyl)-3-(pyridin-3-yl)acrylamide,
N-(6-(2-Cyano-3-pyridin-4-yl)guanidino)hexyl)-N-(3-morpholinopropyl)cyclopentanesulfonamide,
N-(6-(3,4-Dioxo-2-(pyridin-4-ylamino)cyclobut-1-enylamino)hexyl-N-(3-morpholinopropyl)cyclopentanesulfonamide,
N-(3-morpholinopropyl)-N-(6-(3-pyridin-3-ylmethyl)ureido)hexyl)cyclopentanesulfonamide,
(E)-N-(6-(N-(3-morpholinopropyl)cyclopentanesulfonamido)hexyl)-3-(pyridin-3-yl)acrylamide,
N-(3-morpholinopropyl)-N-(6-(3-pyridin-4-ylureido)hexyl)cyclopentanesulfonamide,
N-(3-morpholinopropyl)-N-(6-(3-pyridin-4-ylthioureido)hexyl)cyclopentanesulfonamide,
N-(6-(2-Cyano-3-pyridin-4-yl)guanidino)hexyl)-N-(3-morpholinopropyl)cyclohexanesulfonamide,
N-(6-(3,4-Dioxo-2-(pyridin-4-ylamino)cyclobut-1-enylamino)hexyl-N-(3-morpholinopropyl)cyclohexanesulfonamide,
N-(3-morpholinopropyl)-N-(6-(3-pyridin-3-ylmethyl)ureido)hexyl)cyclohexanesulfonamide,
(E)-N-(6-(N-(3-morpholinopropyl)cyclohexanesulfonamido)hexyl)-3-(pyridin-3-yl)acrylamide,
N-(3-morpholinopropyl)-N-(6-(3-pyridin-4-ylthioureido)hexyl)cyclohexanesulfonamide,
N-(7-(2-Cyano-3-pyridin-4-yl)guanidino)heptyl)-1-phenyl-N-(tetrahydro-2H-pyran-2-yloxy)methanesulfonamide,
N-(7-(3,4-Dioxo-2-(pyridin-4-ylamino)cyclobut-1-enylamino)heptyl)-1-phenyl-N-(tetrahydro-2H-pyran-2-yloxy)methanesulfonamide,
1-Phenyl-N-(7-(3-(pyridin-3-ylmethyl)ureido)heptyl)-N-(tetrahydro-2H-pyran-2-yloxy)methanesulfonamide,
(E)-N-(7-(1-phenyl-N-(tetrahydro-2H-pyran-2-yloxy)methylsulfonamido) heptyl)-3-(pyridin-3-yl)acrylamide,
1-phenyl-N-(7-(3-pyridin-4-ylthioureido)heptyl)-N-(tetrahydro-2H-pyran-2-yloxy)methanesulfonamide
N-(7-(2-cyano-3-(pyridin-4-yl)guanidino)heptyl)-N-(cyclohexylmethoxy)ethanesulfonamide,
N-(cyclohexylmethoxy)-N-(7-(3,4-dioxo-2-(pyridin-4-ylamino)cyclobut-1-enylamino)heptyl)ethanesulfonamide,
N-(cyclohexylmethoxy)-N-(7-(3-(pyridin-3-ylmethyl)ureido)heptyl)ethanesulfonamide,
(E)-N-(7-(N-(cyclohexylmethoxy)ethylsulfonamido)heptyl)-3-(pyridin-3-yl)acrylamide,
N-(cyclohexylmethoxy)-N-(7-(3-(pyridin-4-yl)ureido)heptyl)ethanesulfonamide,
N-(cyclohexylmethoxy)-N-(7-(3-(pyridin-4-yl)thioureido)heptyl)ethanesulfonamide,
N-(7-(2-cyano-3-(pyridin-4-yl)guanidino)heptyl)-N-(cyclohexyloxy)-4-fluorobenzenesulfonamide,
N-(cyclohexyloxy)-N-(7-(3,4-dioxo-2-(pyridin-4-ylamino)cyclobut-1-enylamino)heptyl)-4-fluorobenzenesulfonamide,
N-(cyclohexyloxy)-4-fluoro-N-(7-(3-(pyridin-3-ylmethyl)ureido)heptyl)benzenesulfonamide,
(E)-N-(7-(N-(cyclohexyloxy)-4-fluorophenylsulfonamido)heptyl)-3-(pyridin-3-yl)acrylamide,
N-(cyclohexyloxy)-4-fluoro-N-(7-(3-(pyridin-4-yl)thioureido)heptyl)benzenesulfonamide,
N-(8-(2-cyano-3-(pyridin-4-yl)guanidino)octyl)-N-(3-morpholinopropyl)benzamide,
N-(8-(3,4-dioxo-2-(pyridin-4-ylamino)cyclobut-1-enylamino)octyl)-N-(3-morpholinopropyl)benzamide,
N-(3-morpholinopropyl)-N-(8-(3-(pyridin-3-ylmethyl)ureido)octyl)benzamide,
(E)-N-(3-morpholinopropyl)-N-(8-(3-(pyridin-3-yl)acrylamido)octyl)benzamide,
N-(3-morpholinopropyl)-N-(8-(3-(pyridin-4-yl)thioureido)octyl)benzamide,
N-(8-(2-cyano-3-(pyridin-3-yl)guanidino)octyl)-N-(3-morpholinopropyl)benzamide,
3-cyclohexyl-1-(3-morpholinopropyl)-1-(8-(3-(pyridin-4-yl)thioureido)octyl)urea,
3-(8-(benzyloxy(ethyl)amino)octylamino)-4-(pyridin-4-ylamino)cyclobut-3-ene-1,2-dione,
N-(3-morpholinopropyl)-N-(7-(3-(pyridin-4-yl)thioureido)heptyl)cyclohexanesulfonamide oxalate,
1-(8-(benzyloxy(ethyl)amino)octyl)-3-(pyridin-4-yl)thiourea oxalate,
1-(8-(benzyloxy(ethyl)amino)octyl)-3-(pyridin-3-ylmethyl)urea oxalate,
1-(8-(benzyloxy(ethyl)amino)octyl)-3-(pyridin-4-yl)urea,
N-(3-morpholinopropyl)-N-(7-(3-(pyridin-4-yl)ureido)heptyl)cyclohexanesulfonamide,
1-(8-(benzyloxy(ethyl)amino)octyl)-2-cyano-3-(pyridin-4-yl)guanidine,
1-(8-(benzyl(ethoxy)amino)octyl)-2-cyano-3-(pyridin-4-yl)guanidine,
2-Cyano-1-(8-(ethyl(2-morpholinoethoxy)amino)octyl)-3-(pyridin-4-yl)guanidine oxalate,
2-Cyano-1-(8-(3-morpholinopropylamino)octyl)-3-(pyridin-4-yl)guanidine,
2-Cyano-1-(8-((dimethylphosphoryl)(3-morpholinopropyl)amino)octyl)-3-(pyridin-4-yl)guanidine,
N-(8-(3,4-dioxo-2-(pyridin-4-ylamino)cyclobut-1-enylamino)octyl)-P,P-dimethyl-N-(3-morpholinopropyl)phosphinic amide,
1-(8-((dimethylphosphoryl)(3-morpholinopropyl)amino)octyl)-3-(pyridin-3-ylmethyl)urea,
(E)-N-(8-((dimethylphosphoryl)(3-morpholinopropyl)amino)octyl)-3-(pyridin-3-yl)acrylamide,
1-(8-((dimethylphosphoryl)(3-morpholinopropyl)amino)octyl)-3-(pyridin-4-yl)thiourea,
1-(8-(2-cyano-3-(pyridin-4-yl)guanidino)octyl)-3-cyclohexyl-1-(morpholinopropyl)urea,
1-(8-(2-cyano-3-(pyridin-4-yl)guanidino)octyl)-1-(3-morpholinopropyl)-3-phenylthiourea,
N-(8-(2-cyano-3-(pyridin-4-yl)guanidino)octyl)-N-(3-morpholinopropyl)hydrazinecarboxamide,
N-(8-(3,4-dioxo-2-(pyridin-4-ylamino)cyclobut-1-enylamino)octyl)-N-(3-morpholinopropyl)hydrazinecarboxamide,
N-(3-morpholinopropyl)-N-(8-(3-(pyridin-3-ylmethyl)ureido)octyl)hydrazinecarboxamide,
N-(7-(2-cyano-3-(pyridin-4-yl)guanidino)heptyl)-N-(2-fluoroethyl)cyclohexansylfonamide,
N-(7-(3,4-dioxo-2-(pyridin-4-ylamino)cyclobut-1-enylamino)heptyl)-N-(2-fluoroethyl)cyclohexanesulfonamide,
N-(2-fluoroethyl)-N-(7-(3-(pyridin-3-ylmethyl)ureido)heptyl)cyclohexanesulfonamide,
(E)-N-(7-(N-(2-fluoroethyl)cyclohexanesulfonamido)heptyl)-3-(pyridin-3-yl)acrylamide,
N-(2-fluoroethyl)-N-(7-(3-pyridin-4-ylthioureido)heptyl)cyclohexanesulfonamide,
N-(7-(2-cyano-3-(pyridin-4-yl)guanidino)octyl)-N-(2-fluoroethyl)cyclohexansylfonamide,
N-(7-(3,4-dioxo-2-(pyridin-4-ylamino)cyclobut-1-enylamino)octyl)-N-(2-fluoroethyl)cyclohexanesulfonamide,
(E)-N-(7-(N-(2-fluoroethyl)cyclohexanesulfonamido)octyl)-3-(pyridin-3-yl)acrylamide,
N-(2-fluoroethyl)-N-(7-(3-pyridin-4-ylthioureido)octyl)cyclohexanesulfonamide,
2-cyano-1-(8-(cyclohexylmethoxyamino)octyl)-3-(pyridin-4-yl)guanidine,
N-(8-(2-cyano-3-(pyridin-4-yl)guanidino)octyl)-N-(cyclohexylmethyloxy)-2,2,2-trifluoromethanesulfonamide,
1-(8-(2-cyano-3-(pyridin-4-yl)guanidino)octyl)-3-cyclohexyl-1-(cyclohexylmethoxy)thiourea,
2-cyano-1-(8-(cyclohexylmethoxyamino)hexyl)-3-(pyridin-4-yl)guanidine,
2-cyano-1-(8-(cyclohexylmethoxyamino)heptyl)-3-(pyridin-4-yl)guanidine,
N-(6-(2-cyano-3-(pyridin-4-yl)guanidino)hexyl)-N-(cyclohexylmethoxy)methanesulfonamide,
N-(6-(2-cyano-3-(pyridin-4-yl)guanidino)hexyl)-N-(cyclohexylmethoxy)-2,2,2-trifluoroethanesulfonamide,
1-(6-(2-cyano-3-(pyridin-4-yl)guanidino)hexyl)-1-(cyclohexylmethoxy)-3-ethylurea,
1-(6-(2-cyano-3-(pyridin-4-yl)guanidino)hexyl)-1-(cyclohexylmethoxy)-3-isopropylurea,
1-(6-(2-cyano-3-(pyridin-4-yl)guanidino)hexyl)-1-(cyclohexylmethoxy)-3-methylthiourea,
1-(6-(2-cyano-3-(pyridin-4-yl)guanidino)hexyl)-3-cyclohexyl-1-(cyclohexylmethoxy)thiourea,
N-(7-(2-cyano-3-(pyridin-4-yl)guanidino)heptyl)-N-(cyclohexylmethoxy)methanesulfonamide,
N-(7-(2-cyano-3-(pyridin-4-yl)guanidino)heptyl)-N-(cyclohexylmethoxy)-2,2,2-trifluoroethanesulfonamide,
1-(7-(2-cyano-3-(pyridin-4-yl)guanidino)heptyl)-N-(cyclohexylmethoxy)-3-ethylurea,
1-(7-(2-cyano-3-(pyridin-4-yl)guanidino)heptyl)-N-(cyclohexylmethoxy)-3-isopropylurea,
1-(7-(2-cyano-3-(pyridin-4-yl)guanidino)heptyl)-N-(cyclohexylmethoxy)-3-methylthiourea,
1-(7-(2-cyano-3-(pyridin-4-yl)guanidino)heptyl)-3-cyclohexyl-1-(cyclohexylmethoxy)thiourea,
N-(8-(2-cyano-3-(pyridin-4-yl)guanidino)octyl)-N-(cyclohexylmethoxy)methanesulfonamide,
1-(8-(2-cyano-3-(pyridin-4-yl)guanidino)octyl)-N-(cyclohexylmethoxy)-3-ethylurea,
1-(8-(2-cyano-3-(pyridin-4-yl)guanidino)octyl)-N-(cyclohexylmethoxy)-3-isopropylurea,
1-(8-(2-cyano-3-(pyridin-4-yl)guanidino)octyl)-N-(cyclohexylmethoxy)-3-methylthiourea,
N-(6-(2-cyano-3-(pyridin-4-yl)guanidino)hexyl)-N-(2-fluoroethyl)cyclohexansylfonamide,
N-(6-(3,4-dioxo-2-(pyridin-4-ylamino)cyclobut-1-enylamino)hexyl)-N-(2-fluoroethyl)cyclohexanesulfonamide,
(E)-N-(6-(N-(2-fluoroethyl)cyclohexanesulfonamido)hexyl)-3-(pyridin-3-yl)acrylamide,
N-(2-fluoroethyl)-N-(6-(3-pyridin-4-ylthioureido)hexyl)cyclohexanesulfonamide,
2-cyano-1-(7-morpholinoheptyl)-3-(pyridin-4-yl)guanidine,
3-(7-morpholinoheptylamino)-4-(pyridin-4-ylamino)cyclobut-3-ene-1,2-dione,
1-(7-morpholinoheptylamino)-3-(pyridin-3-ylmethyl)urea,
(E)-N-(7-morpholinoheptyl)-3-(pyridin-3-yl)acrylamide,
1-(7-morpholinoheptyl)-3-(pyridin-4-yl)thiourea,
N-(6-(2-cyano-3-(pyridin-4-yl)guanidino)hexyl)-N-(cyclohexylmethoxy)propane-2-sulfonamide,
N-(6-(2-cyano-3-(pyridin-4-yl)guanidino)hexyl)-N-(cyclohexylmethoxy)ethane-2-sulfonamide,
N-(6-(2-cyano-3-(pyridin-4-yl)guanidino)hexyl)-N-(cyclohexylmethoxy)cyclopropane-2-sulfonamide,
N-(6-(2-cyano-3-(pyridin-4-yl)guanidino)hexyl)-N-(cyclohexylmethoxy)-1,1,1-trifluoromethanesulfonamide,
2-cyano-1-(5-(cyclohexylmethoxyamino)pentyl)-3-(pyridin-4-yl)guanidine,
3-(5-(cyclohexylmethoxyamino)pentylamino)-4-(pyridin-4-ylamino)cyclobut-3-ene-1,2-dione,
N-(5-(2-cyano-3-(pyridin-4-yl)guanidino)pentyl)-N-cyclohexylmethoxy)methanesulfonamide,
N-(5-(2-cyano-3-(pyridin-4-yl)guanidino)pentyl)-N-cyclohexylmethoxy)ethanesulfonamide,
1-(5-(2-cyano-3-(pyridin-4-yl)guanidino)pentyl)-1-(cyclohexylmethoxy)-3-isopropylurea,
1-(5-(2-cyano-3-(pyridin-4-yl)guanidino)pentyl)-1-(cyclohexylmethoxy)-3-ethylurea,
1-(5-(2-cyano-3-(pyridin-4-yl)guanidino)pentyl)-1-(cyclohexylmethoxy)-3-methylthiourea,
N-(5-(2-cyano-3-(pyridin-4-yl)guanidino)pentyl)-N-(cyclohexylmethoxy)benzenesulfonamide,
N-(5-(2-cyano-3-(pyridin-4-yl)guanidino)pentyl)-N-(cyclohexylmethoxy)propane-2-sulfonamide,
N-(benzyloxy)-N-(8-(3-(pyridin-4-yl)ureido)octyl)methanesulfonamide,
N-(benzyloxy)-N-(8-(3-(pyridin-4-yl)thioureido)octyl)methanesulfonamide,
N-(benzyloxy)-N-(6-(2-cyano-3-(pyridin-4-yl)guanidino)hexyl)methanesulfonamide,
N-(benzyloxy)-N-(6-(3,4-dioxo-2-(pyridin-4-ylamino)cyclobut-1-enylamino)hexyl)methanesulfonamide,
N-(benzyloxy)-N-(6-(3-(pyridin-3-ylmethyl)ureido)hexyl)methanesulfonamide,
N-(benzyloxy)-N-(6-(3-(pyridin-4-yl)thioureido)hexyl)methanesulfonamide,
(E)-N-(benzyloxy)methylsulfonamido)hexyl)-3-(pyridin-3-yl)acrylamide,
N-(benzyloxy)-N-(6-(3-(pyridin-4-yl)ureido)hexyl)methanesulfonamide,
N-(benzyloxy)-N-(6-(2-cyano-3-(pyridin-4-yl)guanidino)heptyl)methanesulfonamide,
N-(benzyloxy)-N-(6-(3,4-dioxo-2-(pyridin-4-ylamino)cyclobut-1-enylamino)heptyl)methanesulfonamide,
N-(benzyloxy)-N-(6-(3-(pyridin-3-ylmethyl)ureido)heptyl)methanesulfonamide,
N-(benzyloxy)-N-(6-(3-(pyridin-4-yl)thioureido)heptyl)methanesulfonamide,
(E)-N-(benzyloxy)methylsulfonamido)heptyl)-3-(pyridin-3-yl)acrylamide,
N-(benzyloxy)-N-(6-(3-(pyridin-4-yl)ureido)heptyl)methanesulfonamide,
N-(8-(2-cyano-3-pyridin-4-yl)guanidino)octyl)-N-(4-fluorobenzyloxy)methanesulfonamide,
N-(8-(3,4-dioxo-2-(pyridin-4-ylamino)cyclobute-1-enylamino)octyl)-N-(4-fluorobenzyloxy)methansulfonamide,
N-(4-fluorobenzyloxy)-N-(8-(3-pyridin-3-ylmethyl)ureido)octyl)methansulfonamide,
N-(8-(N-(4-fluorobenzyloxy)methylsulfonamido)octyl-3-(pyridin-3-yl)acrylamide,
N-(4-fluorobenzyloxy)-N-(8-(3-(pyridin-4-yl)ureido)octyl)methanesulfonamide, and
N-(4-fluorobenzyloxy)-N-(8-(3-(pyridin-4-yl)thioureido)octyl)methanesulfonamide.

13. The compound according to any one of the preceding claims for use as a medicament.

14. The compound according to any one of the claims 1-12 for use as a medicament for the treatment of a disease or a condition caused by an elevated level and/or elevated activity of nicotinamide phosphoribosyltransferase (NAMPRT).

15. The compound according to claim 14, wherein said disease or condition is one or more selected from the group consisting of inflammatory and tissue repair disorders, particularly diabetic nephropathy, rheumatoid arthritis, inflammatory bowel disease, asthma and CPOD (chronic obstructive pulmonary disease), osteoarthritis, osteoporosis and fibrotic diseases; dermatosis, including psoriasis, atopic dermatitis and ultra-violet induced skin damage; autoimmune diseases including systemic lupus erythematosis, multiple sclerosis, psoriatic arthritis, ankylosing spondylitis, tissue and organ rejection, Alzheimer's disease, stroke, athersclerosis, restenosis, diabetes, glomerulonephritis, cancer, particularly wherein the cancer is selected from breast, prostate, lung, colon, cervix, ovary, skin, CNS, bladder, pancreas, leukaemia, lymphoma or Hodgkin's disease, cachexia, inflammation associated with infection and certain viral infections, including Acquired Immune Deficiency Syndrome (AIDS), adult respiratory distress syndrome, ataxia telengiectasia.

16. A method of inhibiting the enzymatic activity of nicotinamide phosphoribosyl-transferase (NAMPRT) in a mammal, said method comprising the step of administering to said mammal a pharmaceutically relevant amount of a compound as defined in any of claims 1-12.

17. A method of treating a disease or condition caused by an elevated level of nicotinamide phosphoribosyltransferase (NAMPRT) in a mammal, said method comprising the step of administering to said mammal a pharmaceutically relevant amount of a compound as defined in any of claims 1-12.

18. The method according to claim 17, wherein the compound is administered in combination with a DNA damaging agent.

19. The method according to any one of the claims 17-18, wherein said disease or condition is one or more selected from the group consisting of inflammatory and tissue repair disorders, particularly diabetic nephropathy, rheumatoid arthritis, inflammatory bowel disease, asthma and COPD (chronic obstructive pulmonary disease), osteoarthritis, osteoporosis and fibrotic diseases; dermatosis, including psoriasis, atopic dermatitis and ultra-violet induced skin damage; autoimmune diseases including systemic lupus erythematosis, multiple sclerosis, psoriatic arthritis, ankylosing spondylitis, tissue and organ rejection, Alzheimer's disease, stroke, atherosclerosis, restenosis, diabetes, glomerulonephritis, cancer, particularly wherein the cancer is selected from breast, prostate, lung, colon, cervix, ovary, skin, CNS, bladder, pancreas, leukaemia, lymphoma or Hodgkin's disease, cachexia, inflammation associated with infection and certain viral infections, including Acquired Immune Deficiency Syndrome (AIDS), adult respiratory distress syndrome, ataxia telengiectasia.
